(19) United States
(12) Patent Application Publication
(10) Pub. No.: US 2014/0373600 A1
(43) Pub. Date: Dec. 25, 2014

(54) GAS SENSORS AND METHODS OF PREPARATION THEREOF

(71) Applicant: Georgia Tech Research Corporation, Atlanta, GA (US)

(72) Inventors: James Gole, Atlanta, GA (US); William Ivey Laminack, Atlanta, GA (US)

(21) Appl. No.: 14/460,574

(22) Filed: Aug. 15, 2014

Related U.S. Application Data
(63) Continuation-in-part of application No. 14/336,022, filed on Jul. 21, 2014.
Application No. 14/336,022 is a continuation-in-part of application No. 14/262,117, filed on Apr. 25, 2014.
Application No. 14/262,117 is a continuation-in-part of application No. 14/240,691, filed on Feb. 24, 2014.
(60) Provisional application No. 61/867,280, filed on Aug. 19, 2013.
(60) Provisional application No. 61/954,633, filed on Mar. 18, 2014.
(60) Provisional application No. 61/527,294, filed on Aug. 25, 2011.
(60) Provisional application No. 61/820,237, filed on May 7, 2013.

(51) Int. Cl. G01N 33/00; H01L 29/16

(52) U.S. Cl. CPC G01N 33/0027; G01N 33/0004; H01L 29/16; G01N 2033/0095
USPC 73/31.06; 438/49; 257/414

(57) ABSTRACT

Embodiments of the present disclosure include sensors, arrays of conductometric sensors, devices including conductometric sensors, methods of making conductometric sensors, methods of using conductometric gas sensors, and the like.

### CROSS-REFERENCE TO RELATED APPLICATION

This application claims priority to co-pending U.S. Provisional Application entitled “IHSAB PRINCIPLE AND THE EFFECT OF NITROGEN AND SULFUR FUNCTIONALIZATION ON METAL OXIDE DECORATED INTERFACE RESPONSE” having Ser. No. 61/867,280, filed on Aug. 16, 2014, which is incorporated herein by reference.

In addition, this is a continuation-in-part application of co-pending U.S. Non-Provisional application entitled “GAS SENSORS AND METHODS OF PREPARATION THEREOF” having Ser. No. 14/336,022, filed on Jul. 21, 2014, which claims priority to co-pending U.S. Provisional Application entitled “MAGNETICALLY INDUCED ENHANCEMENT OF REVERSIBLY RESPONDING CODUCTORMETRIC SENSORS” having Ser. No. 61/954,633, filed on Mar. 18, 2014, each of which is incorporated herein by reference.

In addition, this is a continuation-in-part application of co-pending U.S. Non-Provisional application entitled “GAS SENSORS AND METHODS OF PREPARATION THEREOF” having Ser. No. 14/262,117, filed on Apr. 25, 2014, which claims priority to U.S. Provisional Application entitled “SOLAR PUMPING SENSING” having Ser. No. 61/820,237, filed on May 7, 2013; in addition, Ser. No. 14/262,117 is a continuation-in-part application of co-pending U.S. Non-Provisional application entitled “GAS SENSORS AND METHODS OF PREPARATION THEREOF” having Ser. No. 14/240,691, filed on Feb. 24, 2014, which is a 35 U.S.C. §371 national stage of PCT Application No. PCT/US2012/051721, entitled “GAS SENSORS AND METHODS OF PREPARATION THEREOF” and filed Aug. 21, 2012, which claims priority to U.S. Provisional Application entitled “NANOSTRUCTURE DRIVEN ANALYTE-INTERFACE ELECTRON TRANSDUCTION” having Ser. No. 61/527,294, filed on Aug. 25, 2011, each of which is incorporated herein by reference.

### BACKGROUND

Porous silicon (PS) has drawn considerable attention for sensor applications. Its luminescence properties, large surface area, and compatibility with silicon based technologies have been the driving force for this technology development. However, there exists a need in the industry to advance sensor technologies.

### SUMMARY

Embodiments of the present disclosure include sensors, arrays of conductometric sensors, devices including conductometric sensors, methods of making conductometric sensors, methods of using conductometric gas sensors, and the like.

One exemplary embodiment of a method, among others, includes: providing a conductometric porous silicon gas sensor including a silicon substrate having a porous silicon layer, wherein a plurality of metal oxide nanostructures are disposed on a portion of the porous silicon layer to provide a fractional coverage on the porous silicon layer, and functionalizing, in situ, the metal oxide nanostructures with nitrogen, sulfide, or thiol to form a in situ functionalized metal oxide nanostructures. In an embodiment, functionalizing, in situ, the metal oxide nanostructures includes exposing a functionalization agent to the metal oxide nanostructures to form the in situ functionalized metal oxide nanostructures. In an embodiment, the functionalization agent is selected from the group consisting of: triethylamine, tributylamine, aryl amines and a combination thereof. In an embodiment, the functionalization agent is selected from the group consisting of: diethyl sulfide, dibutylsufide, dimethylsulfide, and a combination thereof. In an embodiment, the process can be used to form a structure or device of the present disclosure.

One exemplary embodiment of a device, among others, includes: a conductometric porous silicon gas sensor including a silicon substrate having a porous silicon layer, wherein a plurality of in situ functionalized metal oxide nanostructures are on a portion of the porous silicon layer to provide a fractional coverage on the porous silicon layer, wherein the in situ functionalized metal oxide nanostructures are functionalized in situ with nitrogen, sulfide, or thiol, wherein the conductometric porous silicon gas sensor is operative to transduce the presence of a gas into an impedance change, wherein the impedance change correlates to the gas concentration. In an embodiment, the in situ functionalized metal oxide nanostructures are more basic relative to unfunctionalized metal oxide nanostructures. In an embodiment, the in situ functionalized metal oxide nanostructures formed from thiol groups intereacting with and functionalizing in situ, the metal oxide nanostructures.

One exemplary embodiment of a method of detecting a concentration of a gas, among others, includes: providing a conductometric porous silicon gas sensor including a silicon substrate having a porous silicon layer, wherein a plurality of in situ functionalized metal oxide nanostructures are on a portion of the porous silicon layer to provide a fractional coverage on the porous silicon layer, wherein the in situ functionalized metal oxide nanostructures are functionalized in situ with nitrogen, sulfide, or thiol, wherein the conductometric porous silicon gas sensor is operative to transduce the presence of a gas into an impedance change, wherein the impedance change correlates to the gas concentration: introducing the gas to the sensor; and measuring an impedance change in the sensor.

Other devices, systems, methods, features, and advantages of the present disclosure will be or become apparent to one with skill in the art upon examination of the following detailed description. It is intended that all such additional devices, systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1.1 illustrates the estimated hard and soft acidities and basicities based on resistance changes relative to a p-type and n-type porous silicon interface. The acidic metal oxides that decorate the semiconductor interface can be modified through in-situ nitridation, decreasing their Lewis acidity. The analytes remain as positioned. A horizontal line is used to separate the metal oxides used to modify the interface (above) and the analytes below in the figure.

FIGS. 1.2A-C illustrate the response of an untreated PS interface to the analyte gas as a dark grey solid line and the response after nitridation is in a light grey dotted line. The boxes (Black dashed) denote the analyte concentration from 1 to 10 ppm over the time of analyte gas exposure to the sensors. FIG. 1.2A illustrates a graph of a rapid response observed for NH₃ at each concentration. Nitridation of the sensor for 15 seconds produces an increase in the interface response monitored as an increase in conductance (decrease in resistance). FIG. 1.2B illustrates the change in the response to NH₃ after nitridation of the interface for one hour with triethylamine. A rapid response is again observed at each concentration. Nitridation produces an increase in the interface response as monitored as an increase in resistance (decrease in conductance). FIG. 1.2C illustrates the response of an untreated PS interface to NO and after nitridation of the PS interface with trimethylamine for 15 seconds. While a rapid response is observed at each concentration, Nitridation results in a decrease in the interface response as monitored as a decrease in resistance.

FIGS. 1.3A and B illustrate graphs showing boxes (dashed black) that denote the analyte concentration from 1 to 10 ppm over the time of analyte gas exposure to the sensors. A rapid response is observed at each concentration for all sensors. FIG. 1.3A illustrates the response corresponding to decreasing resistance as NH₃ contributes electrons to an untreated porous silicon (PS), TiO₂, and a TiO₂₋ₓNₓ treated PS interface. The TiO₂₋ₓNₓ treated interface is basic relative to the PS and TiO₂ treated PS acidic sites. Nitridation for 15 seconds results in a decrease in the interface response as monitored as a decrease in resistance. FIG. 1.3B illustrates the response of a TiO₂ treated PS interface to NO (gray solid) and after nitridation for 15 seconds with triethylamine (gray dashed). The signal for untreated TiO₂ on the same scale as that for the oxynitride actually bottoms out. Nitridation results in a decrease in the interface response as monitored as a decrease in resistance.

FIGS. 1.4A and B illustrate the response to NH₃ (1.4A) and NO (1.4B) of an SnO₂ treated PS interface before (solid gray) and after (dotted gray) nitridation of the surface for 15 seconds with triethylamine. The boxes (black dashes) denote the analyte concentration from 1 to 10 ppm over the time of analyte gas exposure to the sensors. A rapid response is observed at each concentration. Nitridation results in a decrease in the interface response as monitored as a decrease in conductance for NH₃ and NO.

FIGS. 1.5A-B illustrate the response to NH₃ (1.5A) and NO (1.5B) of an NiO treated PS interface before (solid gray) and after (dotted gray) nitridation of the surface for 15 seconds with triethylamine. The boxes (black dashes) denote the analyte concentration from 1 to 10 ppm over the time of analyte gas exposure to the sensors. A rapid response is observed at each concentration. For NH₃, nitridation produces an increase in the interface response as monitored as an increase in conductance (decrease in resistance); however, for NO the nitridation decreases the interface response.

FIGS. 1.6A and B illustrate boxes (dashed black) that denote the analyte concentration from 1 to 10 ppm over the time of analyte gas exposure to the sensors. A rapid response is observed at each concentration. FIG. 1.6A illustrates the response of a CuₓO treated PS interface to NH₃ (gray solid), after nitridation with triethylamine (gray dotted), and after white light excitation of the nitridated sample (gray dashed dotted). Nitridation produces an increase in the interface response as monitored as an increase in conductance (decrease in resistance). FIG. 1.6B illustrates the response of a CuₓO treated PS interface to NO (gray solid) and after nitridation with triethylamine (gray dashed). Nitridation produces an increase in the interface response as monitored as an increase in conductance (decrease in resistance).

FIG. 1.7A illustrates a Ti 2p XPS spectrum showing a lower binding energy for the nitridated form of TiO₂. FIG. 1.7B illustrates a Sn 3d XPS spectrum showing a lower binding energy for the nitridated form of SnO₂. FIG. 1.7C illustrates a Ni 2p XPS spectrum showing a lower binding energy for the nitridated form of NiO. FIG. 1.7D illustrates a Cu 2p XPS spectrum showing a higher binding energy for the nitridated form of CuₓO.

FIG. 1.8 illustrates a nitrogen 1s XPS spectra associated with the nitridation of TiO₂, SnOₓ, NiO, and CuₓO depositions.

FIG. 1.9A illustrates an oxygen 1s XPS spectra associated with the nitridation of TiO₂ depositions. Both TiO₂ and SiO₂peaks are observed and suggest a shift to higher binding energy on nitridation. FIG. 1.9B illustrate an oxygen 1s XPS spectra associated with the nitridation of SnO₂ depositions. The spectra show a notable red shift on nitridation. FIG. 1.9C illustrates an oxygen 1s XPS spectra associated with the nitridation of NiO, and CuₓO depositions. The data show an increasing and notable shift in binding energy on nitridation.

FIG. 1.10 illustrates a schematic of an in-situ nitridation of metal oxide nanostructures deposited to an n-type extrinsic semiconductor interface.

FIG. 2.1 is a schematic representation of PS sensor system.

FIGS. 2.2A-F illustrate a comparison of responses to 1-5 and 10 ppm NO for (2.2A) an untreated n-type PS micro/nanostructured interface with those treated with (2.2B) TiO₂, (2.2C) SnO₂, (2.2D) NiO, (2.2E) CuₓO, and (2.2F) AuₓO fractional nanostructured island depositions. NO was pulsed onto these interfaces with a 300 s half-cycle w by a 300 s half-cycle nitrogen cleaning. The system was purged with UHP nitrogen for 1800 s before operation.

FIG. 2.3 illustrates a comparison of metal oxide (usually SnO₂ or WO₃) elevated temperature (150-500° C.) heat controlled sensors separated from their electronics by a channel with a heat sunk PS sensor operating at room temperature and capable of operation to temperatures of at least 200° C.

FIG. 2.4 illustrates a response corresponding to decreasing resistance as NH₃ contributes electrons to a CuₓO treated porous silicon and nitridated CuₓO nanostructure treated PS. The nitridated CuₓO treated interface is basic relative to the PS and CuₓO treated PS acidic sites. This interface becomes more acidic upon exposure to white light.

FIG. 2.5 illustrates a response corresponding to decreasing resistance as NH₃ contributes electrons to a TiO₂ treated PS interface without light exposure and the same interface exposed to UV radiation.

FIG. 2.6 illustrates a response corresponding to increased resistance as NO₂ extracts electrons from a TiO₂ treated PS interface without light exposure and the same interface exposed to UV radiation.

FIG. 2.7 illustrates a response corresponding to decreasing resistance as a significant TiO₂₋ₓNₓ fractional nanostructure deposition leads to the extraction of electrons from the moderate acid NO₂.

FIGS. 3.1A-F illustrate a comparison of responses to 1, 2, 3, 4, 5 and 10 ppm NH₃ for (3.1A), (3.1C) and (3.1F), sensors, having an untreated n-type porous silicon (PS) interface with those treated with (3.1B) TiO₂, (3.1D) SnOₓ and (3.1F) NiO fractional nanostructured island depositions. The PS interface in (3.1A) is that treated with TiO₂ in (3.1B) and similarly for SnOₓ and NiO.

FIG. 3.2 illustrates a response of ethanethiol-treated tin oxide nanostructure-deposited porous silicon (PS) interface to NH₃ after exposure for 30 s vs. only tin oxide. The response of the thiol-treated SnOₓ-deposited interface is consistent with the introduction of S—H groups on the interface and an increased Brönsted acidity relative to the PS— and SnOₓ-treated PS acidic sites (FIG. 1.1) after a 30 s exposure.

FIG. 3.3 illustrates a response of ethanethiol-treated nickel oxide nanostructure-deposited porous silicon (PS) interface to NH₃ exposure for 30 s, and exposure only to nickel oxide. The thiol-treated NiO-deposited interface is more acidic than the NiO-treated PS acidic sites (FIG. 3.1) after a 30 s exposure.

FIG. 3.4 illustrates a response corresponding to decreasing resistance as NH₃ contributes electrons to a CuₓO-treated porous silicon and nitridated CuₓO nanostructure-treated PS (green line). The nitridated CuₓO-treated interface is basic relative to the PS— and CuₓO-treated PS acidic sites.

FIG. 3.5 illustrates a response of a CuₓO-treated PS interface to NO (blue line) and after nitridation with triethylamine. The boxes denote the analyte concentration from 1 to 10 ppm.

FIG. 3.6 illustrates a response of diethyl sulfide, Et₂S, -treated TiO₂-deposited porous silicon (PS) interface to NH₃. Exposure to TiO₂-treated PS interface. Response of diethyl sulfide-treated TiO₂ nanostructure-deposited PS interface to NH₃. The Et₂S-treated TiO₂ deposited interface is made more basic relative to the PS and TiO₂-treated PS acidic sites.

FIG. 3.7 illustrates a response of diethyl sulfide, Et₂S, -treated tin oxide-deposited porous silicon (PS) interface to NH₃. Exposure to SnOₓ-treated PS interface. Initial exposure to Et₂S with water present. Response of diethyl sulfide-treated tin oxide nanostructure-deposited PS interface to NH₃ after gentle heating to 80° C. to remove water. The Et₂S treated SnOₓ-deposited interface is a weaker Lewis acid relative to the PS and SnOₓ-treated PS acidic sites, where it is more acidic in the presence of water (See, also, the thiol results).

FIG. 3.8 illustrate a response of diethyl sulfide-treated nickel oxide nanostructure-deposited porous silicon (PS) interface to NH₃. Initial response of nickel oxide-treated PS, after treatment for 10 s with diethyl sulfide, and after treatment for 15 s with diethyl sulfide. The Et₂S-treated NiO-deposited interface treated for 15 s is a weaker Lewis acid made more basic relative to the PS— and NiO-treated (FIG. 3.1) PS interface

FIG. 4.1 illustrates a response corresponding to decreasing resistance as NH₃ contributes electrons to a CuₓO treated porous silicon and nitridated CuₓO nanostructure treated PS. The nitridated CuₓO treated interface is basic relative to the PS and CuₓO treated PS acidic sites.

FIG. 4.2A-F illustrates a comparison of responses to 1-5 and 10 ppm NO for (4.2A) an untreated n-type PS micro/nanostructured interface with those treated with (4.2B) TiO₂, (4.2C) SnO₂, (4.2D) NiO, (4.2E) CuₓO, and (4.2F) AuₓO fractional nanostructured island depositions. No was pulsed onto these interfaces with a 300 s half-cycle followed by a 300 s half-cycle nitrogen cleaning. The system was purged with UHP nitrogen for 1800 s before operation.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that any pair of intervening values in the stated range are encompassed within the disclosure although they may not be specifically recited, subject to any specifically excluded limit in the stated range (e.g., if the range is 1 to 100, then the range of 10 to 20 is also included and can be claimed even if not specifically recited).

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, and the like, which are within the skill of the art. Such techniques are explained fully in the literature.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

The term “detectable” refers to the ability to detect a signal over the background signal.

The term “detectable signal” is a signal derived from an impedance change upon the interaction of a gas with a porous silicon layer or a porous silicon layer having a nanostructured deposit on the porous silicon layer. The detectable signal is detectable and distinguishable from other background signals. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, 40% or more difference between the detectable signal and the background) between the detectable signal and the background. Standards and/or calibration curves and/or arrays of porous silicon sensors can be used to determine the relative intensity of the detectable signal and/or the background.

### Discussion

Embodiments of the present disclosure include sensors, arrays of sensors, devices including sensors, methods of making sensors, methods of using sensors, and the like. In an embodiment, the present disclosure includes porous silicon (PS) sensors, arrays of PS sensors, devices including PS sensors, methods of making PS sensors, methods of using PS gas sensors, and the like.

Embodiments of the present disclosure can be understood based on the Inverse Hard/Soft Acid/Base (IHSAB) principle/approach. The Inverse Hard/Soft Acid/Base (IHSAB) principle/approach, as it correlates with a basis in physisorption (electron transduction), complements the HSAB principle for hard/soft acid/base interactions and can be correlated with a basis in density functional theory (DFT). The basis for correlation follows the principle that soft-soft acid/base interactions produce significant covalent bonding and hard-hard combinations produce significant ionic bonding. The HSAB principle states that hard acids prefer to coordinate to hard bases and soft acids prefer to coordinate to soft bases. In contrast, the driving principle to promote physisorption (electron transduction) represents the inverse of that necessary to form strong chemical bonds. This principle is manifest through the interactions of metal oxide nanostructures as they influence the nature of the majority carriers in a p- or n-type semiconductor. In an embodiment, nanoporous silicon layers positioned on porous silicon (PS) micropores facilitate the deposition of nanostructured metal/metal oxides which provide distinctly higher variable sensitivities and selectivity for a given extrinsic semiconductor interface. In an embodiment, the PS surface is treated with fractional depositions (e.g., islands of nanostructures), much less than a monolayer to insure that the nanostructures do not begin to cross talk and, in so doing diminish signal to noise. This deposition can be made to produce a dominant physisorptive (sensors) or chemisorptive (micro-reactors) character at the semiconductor interface as the deposited nanostructures act to focus the nature of the surface interaction. However, the nature of the extrinsic semiconductor, the manner in which its donor and/or acceptor levels can be manipulated, and its transformation to intrinsic character also represent important variables.

An embodiment of the present disclosure describes a conductometric gas sensor that is operative to measure an impedance change that corresponds to a gas concentration (e.g., a gas concentration can be determined based on the impedance change or the magnitude of the impedance change and this concentration can be independently evaluated for calibration). More particularly, the conductometric sensor transduces the presence of a gas into an impedance signal, which is measured by another device in communication with the conductometric sensor. Therefore, the term “measure” used in reference to the conductometric sensor can include the conductometric sensor in combination with another circuit or device (e.g., impedance analyzer, sensor and shunt circuit, and the like) to measure the impedance (e.g., the detectable signal). The conductometric sensor can be used to detect gases or liquids. In particular, conductometric sensors, in accordance with the present disclosure, have a rapid and reversible response to analyte gases at room temperature.

In an embodiment, the n-type semiconductor is PS, so the sensor can be referred to as a conductometric PS gas sensor (also referred to as a “PS gas sensor” or “conductometric PS sensor”). Although some embodiments of the present disclosure are described as PS, other n-type semiconductor materials can be used and include a porous layer. There is no intention to limit embodiments of the present disclosure to PS materials and other materials, such as those described herein, can be used.

In an embodiment, the conductometric PS gas sensor can be made from an n-type PS substrate. In an embodiment, the conductometric PS gas sensor can be made from a p-type PS substrate. In an embodiment, a conductometric PS gas sensor can operate at a concentrations that can extend down to the lowest parts per billion (ppb) less than 100 ppb, less than about 75 ppb, less than about 50 ppb, less than about 35 ppb, about 1 to 100 ppb, about 1 to 75 ppb, about 1 to 50 ppb, about 1 to 35 ppb. In an embodiment, both p-type and n-type PS based conductometric PS gas sensors that can be used and, in some instances, in a wide dynamic range of gas concentrations. Some detection abilities for some specific gases are given in the Examples, and these illustrate the general sensitivity of the sensor which can be extended to other gases or gas mixtures.

Embodiments of the present disclosure provide for a concept that is predictive of significant and predictable changes in the conductometric sensor (e.g., conductometric PS sensor) sensitivity for a variety of gases. Rapidly responding, reversible, sensitive, and selective conductometric PS sensors can be formed (1) with a highly efficient electrical contact to a porous silicon layer (e.g., a nanopore covered microporous layer), while also including (2) deposited nanostructures (e.g., nanostructures as nanostructured fractional deposits, which enhance the sensitivity of the sensor), using embodiments of the present disclosure.

In an embodiment, the in situ functionalized metal oxide nanostructures can be deposited as distinctly variable nanostructures that can be chosen to be deposited on a portion (e.g, distinct islands) of the porous silicon layer, where the resulting conductometric PS sensor provides a range of sensitivities for a given analyte using a concept complementary to that of hard and soft acid-base interactions (HSAB) and commensurate with a basis in dominant physisorption. The physisorption interaction involves electron transduction between the gas and in situ functionalized metal oxide nanostructure deposits. The physisorption interaction may involve a change in the electronic orbital patterns of the in situ functionalized metal oxide nanostructure deposit but the key is the analyte gas and orbital miss-match which leads to weak interation. A physisorption interaction is a reversible interaction of the in situ functionalized metal oxide nanostructure deposit with the gas. A physisorption interaction is not a chemisorption reaction that involves a chemical reaction that may not be reversible. The concept, based on the reversible interaction of hard acids and bases with soft bases and acids corresponds (1) to the inverse of the HSAB concept and (2) to the selection of a conductometric PS sensor and a porous silicon surface (e.g., in situ functionalized metal oxide nanostructure islands) and analyte materials, which do not undergo strong covalent or ionic bonding but rather represent a much weaker orbital interaction where a significant HOMO-LUMO and additional orbital mismatches dominate the interaction as a reversible physisorption interaction (electron transduction). For example, at 300° K for an n-type semiconductor, the donor level population has been depleted sufficiently so that there are a significant number of levels available for population when a basic analyte interacts with the decorated semiconductor interface, contributes electrons to the donor levels, enhances the majority charge carrier concentration, and increases conductance. This process can eventually “top out” the level population. Similarly, an acidic analyte, as it withdraws electrons, decreases conductance, depletes and can eventually “bottom out” the donor level population. Embodiments of the present disclosure provide for notably higher sensitivities and selectivity based on impedance changes.

An embodiment of the present disclosure can be advantageous for one or more of the following reasons: (1) its operation at room temperature as well as at a single, readily accessible, temperature with an insensitivity to temperature drift, (2) its potential operation in a heat-sunk configuration allowing operation to a surface temperature of 80° C. even in highly elevated temperature environments (in sharp contrast to metal oxide sensors), (3) its ease of deposition with a diversity of gas-selective materials to form sensor arrays, (4) its low cost of fabrication, (5) its low cost and ease of rejuvenation after contamination, (6) its low cost of operation, and/or (7) its ability to rapidly assess false positives by operating the sensor in a pulsed gas mode.

It should be noted that the time frame for measuring the gas concentration depends, in part, on the experimental configuration for gas delivery and the particular application and gas being measured. The presence of the gas can be measured in a time frame less than or equal to 2 seconds in some embodiments which can be dictated by the gas delivery system, while in other embodiments, the time frame for a precise concentration measurement may be longer. In an embodiment, the sensor itself responds in less than 2 seconds. It should be noted that impedance includes contributions from one or more of resistance, capacitance, and inductance, and measurement of impedance includes the measurement of one or more of resistance, capacitance, and inductance. In an embodiment, the impedance analyzer measures the resistance and capacitance only.

Embodiments of the present disclosure can be used to measure the concentration of a gas or a mixture of gases when the gas or the gas mixture is known or substantially known. For example, if the environment that the conductometric PS sensor is to be used in is known to include ammonia, the conductometric PS sensor can be used to measure the concentration of ammonia. In another example, if the environment that the conductometric PS sensor is to be used is known to include ammonia and H₂S, then an array matrix of conductometric PS sensors (e.g., 2, 3, 4, 6, 8, or more) can be used to measure the concentration of each gas or the relative concentration of each gas. In an embodiment, the array of conductometric PS sensors can be designed so that each of the conductometric PS sensors has a greater sensitivity for detection of a specific gas. In addition, the array of conductometric PS sensors allows the concentration of the gases to be compared to one another on relative terms.

In an embodiment, the conductometric PS sensor includes a silicon substrate (n- or p-type), a protective layer on a portion of the silicon substrate, an n- or p-type PS layer (or region) on a portion of the silicon substrate that is not covered by the protective layer, and two or more distinct gold contacts disposed onto a portion of the PS region and the protective layer. A plurality of in situ functionalized metal oxide nanostructures (sometimes referred to as a “in situ functionalized metal oxide nanostructured deposits”) can be disposed in a fractional manner (e.g., non-contiguous islands) on and/or within the n-type PS layer that is not covered by the contacts, which enables the conductometric PS sensor to respond more strongly to certain gases relative to others depending on the nanostructures used.

In an embodiment, the protective layer can include, but is not limited to, a silicon carbide layer, a silicon nitride layer, a silicon oxynitride (SiOₓN_{y}) layer, an insulating dielectric film, a ceramic layer, and combinations thereof. In an effort to be clear, the protective layer may be referred to as the silicon carbide layer hereinafter, but the protective layer could be any one of the layers described above in other embodiments.

In an embodiment, the n- or p-type PS layer can include a macroporous/nanoporous hybrid framework. The nanopores are superimposed on the walls of the macropores. In an embodiment, the macropores can be about 0.5 to 20 μm deep and about 1 to 3 μm in diameter. In an embodiment, the nanopores can be about 1 to 20 nanometers in diameter.

In an embodiment, the contact can be disposed on and within the macroporous and nanoporous hybrid framework as well as extend above the n- or p-type PS layer and onto the protective layer (e.g., silicon carbide layer). In other words, the material fills in a portion of the n- or p-type PS layer and then forms a layer on top of the n- or p-type PS layer. The contacts are distinct and separated from one another by a space or area (e.g., a portion of the PS layer and a portion of the protective layer). In an embodiment, the contact can include one or more contact portions. In other words, one portion can be disposed on the n- or p-type PS layer and one portion disposed on the protective layer, but the two portions are contiguous in that a single metal layer extends from the n-type PS layer onto the top of the n- or p-type PS layer and onto the protective layer. The contacts can be made of a metal or a combination of metals such as, for example, gold. In an embodiment, the contact includes a pre-coating layer usually titanium, and a metal layer usually gold, disposed onto the pre-coating layer. The pre-coating layer can be used to improve the electrical connection of the contact to the n- or p-type PS layer.

As mentioned above, the exposed portion of the n- or p-type PS layer not covered by the contacts can have a plurality of in situ functionalized metal oxide nanostructures positioned (e.g., nanostructure islands) on and/or within the n- or p-type PS layer (e.g., a combined macroporous/nanoporous hybrid framework). The in situ functionalized metal oxide nanostructures can include, but are not limited to, a nitrogen substituted metal oxide nanostructure, a sulfur substituted metal oxide nanostructure, a sulfide substituted metal oxide nanostructure, a thiol metal oxide nanostructure, and combinations thereof, where the substitution is for a portion of the oxygen in the metal oxide or the a bond can be formed to the oxygen. In an embodiment, the in situ functionalized metal oxide nanostructures can be discrete and/or clustered nanostructures and/or nanomaterials on and/or with the n- or p-type PS layer. In another embodiment, the in situ functionalized metal oxide nanostructures can be deposited onto and/or within discrete areas of the n- or p-type PS layer.

In an embodiment, the nanostructure can include a nanoparticle such as a nanosphere. In an embodiment, the nanostructure could be a nanowire, a nanodisk, or a nanobelt. In an embodiment, the nanoparticle can be uncoated or coated.

In an embodiment, the nanostructure can be made of silicon (Si), tin (Sn), chromium (Cr), iron (Fe), nickel (Ni), silver (Ag), titanium (Ti), cobalt (Co), zinc (Zn), platinum (Pt), palladium (Pd), osmium (Os), gold (Au), lead (Pb), iridium (Ir), rhodium (Rh), ruthenium (Ru), molybdenum (Mo), vanadium (V), and aluminum (Al), or oxides thereof. In an embodiment, the nanostructure can be made of metals that can be transformed in situ to form the metal oxide nanostructure. In an embodiment, the nanostructure can be made of: aluminum oxide (Al₂O₃, AlOₓ, x is 1 to 2), silicon oxide (SiOₓ, x is 1 to 4), tin oxide (SnOₓ, x is 2 to 4), chromia (Cr₂O₃), iron oxide (Fe₂O₃, Fe₃O₄, or FeO), nickel oxide (NiO), silver oxide (AgO), cobalt oxide (Co₂O₃, Co₃O₄, or CoO), zinc oxide (ZnO), platinum oxide (PtO), palladium oxide (PdO), vanadium oxide (VO₂), molybdenum oxide (MoO₂), lead oxide (PbO), titanium oxide (TiO₂), clustered oxides of each of these, and a combination thereof. In an embodiment, the metal oxide nanostructures can be functionalized, in situ, to form the in situ functionalized metal oxide nanostructure that includes O, N, thiol (e.g. —S—H—R, where R can be an alkyl group (e.g, one to six carbon alkyl group)), or sulfide (S—R), where N and the sulfide can replace some of the O in the metal oxide and/or be bonded to the metal oxide. Using a variety metal oxide materials, the conductometric PS sensor can be designed to provide selectivity for a particular gas.

In an embodiment, each in situ functionalized metal oxide nanostructure, a group of in situ functionalized metal oxide nanostructures, or a cluster of in situ functionalized metal oxide nanostructures, form an island on the n-type PS layer. In an embodiment, the islands are spaced apart so there is no cross talk between the islands that would impede, substantially impede (e.g., impede about 50% or more, about 75% or more, about 90% or more, or about 50 to 90%, relative to no impedance), or substantially interfere (e.g., interfere about 80% or more, about 90% or more, or about 95% or more, relative to no interference) with the detection of the gas(es) of interest.

Embodiments of the present disclosure also include methods of making conductometric PS sensors. In general, the conductometric PS sensor can be fabricated by first providing an n- or p-type silicon substrate (or other appropriate substrate) having a protective layer, such as a silicon carbide layer, disposed on a first portion of the silicon substrate. Then, a first area on the silicon substrate is converted into an n- or p-type PS layer, where the first area does not have a silicon carbide layer disposed thereon. Next, a first contact (e.g., a first contact pre-coating (e.g., Ti or Cr) and the first contact (e.g., Au)) is formed onto a first portion of the n- or p-type PS layer and onto a first portion of the silicon carbide layer. The first portion of the silicon carbide layer is contiguous with the first portion of the n- or p-type PS layer as described above. A second contact (e.g., a second contact pre-coating (e.g., Ti or Cr) and the second contact (e.g., Au)) is formed onto a second portion of the n- or p-type PS layer and onto a second portion of the silicon carbide layer. The second portion of the silicon carbide layer is contiguous with the second portion of the n- or p-type PS layer as described above. A third portion of the n- or p-type PS layer is between the contacted first portion and the second portion of the n- or p-type PS layer. The first and second contacts can be formed at the same time. Specifically, the first and the second contact pre-coatings are formed and then the first and second contacts are formed on the first and second contact pre-coatings, respectively. The first and second contact pre-coatings are advantageous in that the first and second contacts enable a superior electrical connection to be formed. In addition, the first and second contacts can be formed using a shadow mask technique.

In an embodiment, the silicon substrate can be replaced with any extrinsic n- or p-type semiconductor (e.g., GaP, InP, CdTe, and the like) onto which a porous microstructure can be generated. In an embodiment, the silicon substrate can include wafers, such as, but not limited to, n-, p, or p⁺-type doped wafers, for example n-type phosphorous doped wafers. In an embodiment, the silicon substrate can include wafers, such as, but not limited to, n-, p, or p⁺-type doped silicon wafers, n-type phosphorous doped silicon wafers.

In an embodiment, other materials can be used in place of the silicon carbide layer such as, but not limited to, a silicon nitride layer, a SiOₓN_{y} layer, an insulating dielectric film, and a ceramic layer.

In an embodiment, the p-type PS layer can be prepared by electrochemically etching a portion of the silicon substrate with acetonitrile, hydrofluoric acid, tetrabutylammonium-perchlorate (TBAP), and water, for example. Additional details regarding the preparation of the n-type PS layer are presented in more detail herein.

In an embodiment, the first contact pre-coating layer and the second contact pre-coating layer can be made from titanium or chromium, for example, and can be about 100 to 300 angstroms thick or about 200 angstroms thick. The first contact pre-coating and the second contact pre-coating can be disposed onto the two portions of the PS substrate via techniques such as, but not limited to, electron-beam evaporation, sputtering, electroless plating, and electroplating.

In an embodiment, a second contact can be made of metals, such as, but not limited to, gold (Au), and can be about 1000 to 4000 angstroms thick or about 3000 to 5000 angstroms thick to facilitate wire bonding. The second contact can be disposed onto the two portions of the PS substrate via techniques such as, but not limited to, electron-beam evaporation, sputtering, electroless plating, and electroplating.

One or more types of metal nanostructures can be fractionally disposed on an n- or p-type PS layer, where the nanostructure(s) form islands on the doped PS layer. In an embodiment, the nanostructures can be disposed using techniques such, but not limited to, depositions from solutions including depositions from electroless solutions, and depositions from sol-gel solutions; electron-beam evaporation; sputtering; electroless plating; and electroplating.

Subsequently, the metal nanostructures can be formed into metal oxide nanostructures in situ. Alternatively, the nanostructures disposed to the PS layer are metal oxide nanostructures. Specific embodiments and specific details are provided in the Examples.

After formation of the metal oxide nanostructures, the metal oxide nanostructures can be functionalized in situ to form the in situ functionalized metal oxide nanostructures. As mentioned above, the in situ functionalizing of the metal oxide nanostructures can include functionalization with nitrogen or sulfur compounds (e.g, sulfides, thiols) to form in situ functionalized metal oxide nanostructures. In an embodiment, functionalizing includes forming thiol compounds of the sulfides or thiols that maintain (e.g., exist after functionalization of the precursor compound) S—R or S—H—R groups on the surface of the metal oxide nanostructures. In an embodiment, relative to the metal oxide nanostructures, the in situ functionalized (e.g., with N or S or SR) metal oxide nanostructures can have a more basic character, which alters the conductormetric response to the same gas.

In an embodiment, the in situ functionalized metal oxide nanostructures include thiol-based groups on the surface of the metal oxide nanostructures. In an embodiment, relative to the metal oxide nanostructures, the in situ functionalized (e.g., with thiol group) metal oxide nanostructures can have a more acidic character, which alters the conductormetric response to the same gas.

In an embodiment, the functionalization can include using a functionalization agent. In this regard, the metal oxide nanostructures are exposed to the functionalization agent for about 5 to 60 seconds or 10 to 30 seconds at a temperature of about room temperature. Specific embodiments and specific details regarding functionalization are provided in the Examples.

In an embodiment, the functionalization agent can include a compound that functions to substitute or bond to an oxygen with nitrogen or sulfur or sulfur-based moieties from sulfides or thiols (e.g., S—R or S—H—R groups) interacting with the metal oxide. In an embodiment, the functionalization agent can include triethylamine, an alternate amine or aryl group, or a combination thereof. In an embodiment, the functionalization agent can include diethyl sulfide, dibutyl sulfide, or a combination thereof. In an embodiment, the functionalization agent can include ethane thiol, butane thiol, or a combination thereof.

Additional fabrication steps can be conducted. For example, an additional fabrication step includes cleaning the n- or p-type PS layer with a mixture of one part hydrochloric acid (e.g., about 44%) in about five parts methanol for about four hours.

After the conductometric PS sensor is formed, the conductometric PS sensor can be validated. In this regard, embodiments of this disclosure include methods of selecting a conductometric PS sensor having certain performance characteristics, methods of analyzing the data measured using the conductometric PS sensor, and methods of measuring the concentration of a gas. In addition, the method of validating includes detecting false positives (e.g., determining that an impedance change is from the gas of interest and not a response caused by another source). Furthermore, the present disclosure provides methods of analyzing data for the conductometric PS sensor as well as for other devices and sensors.

Embodiments of the conductometric PS sensor having a plurality of in situ functionalized metal oxide nanostructures on the n- or p-type PS layer can provide enhanced sensitivity and selectivity to certain gases. In particular, concentrations of select gases can be measured in the presence with one or more additional gases, where selected gases are more strongly sensed (e.g., impedance change detected). In an embodiment, the selectivity to one gas over one or more others can be controlled by selection of the type or combination of types of in situ functionalized metal oxide nanostructures.

As briefly mentioned above, the conductometric PS sensor responds and is operative to measure an impedance change (e.g., an impedance magnitude change) across a first contact and a second contact that corresponds to a concentration of a gas in contact with the PS surface. The sensitivity of the conductometric PS sensor is defined as the relative increase or decrease in impedance over a time frame following exposure to a concentration of a gas of interest. It should also be noted that the sensitivity is, in part, a function of the analyte gas of interest, the nature of the in situ functionalized metal oxide nanostructure deposits, and whether the extrinsic semiconductor is n- or p-type.

The operating parameters of the conductometric PS sensor include, but are not limited to, a bias voltage, an AC voltage frequency, an AC voltage amplitude and combinations thereof. The conductometric PS sensors operate at a bias voltage between about 10 and 3000 millivolts DC. Also one can use an AC voltage frequency between 100 and 100,000 Hz, and an AC voltage amplitude between 1 and 1000 millivolts, or a combination thereof.

As mentioned briefly above, the impedance change can be measured with an impedance analyzer, a sensor and shunt circuit, or other impedance measurement devices. An embodiment of the sensor and shunt circuit uses a high impedance resistor in parallel with the sensor (conductometric PS sensor or components thereof). The resistor shunts the stray capacitance (removes high frequency noise), resulting in a resistive measurement.

The conductometric PS sensor can be used in a variety of ways including, but not limited to, a stand-alone detector device or system, or a device or system including an array of stand-alone detectors (e.g., one or more types of n-type conductometric PS sensors and one or more types of p-type conductometric PS sensors). The conductometric PS sensor can be used to detect gases (e.g., combustion generated gases such as carbon monoxide, carbon dioxide, sulfur dioxides, nitrogen oxides, and hydrogen sulfide). In particular, conductometric PS sensors, in accordance with the present disclosure, can provide a rapid and reversible response to analyte gases (e.g., including hydrogen chloride (HCl), ammonia (NH₃), phosphine (PH₃), carbon monoxide (CO), sulfur dioxide (SO₂), hydrogen sulfide (H₂S) nitric oxide (NOₓ), and toluene) at room temperature. Additional details regarding analyte gases are described in the Examples.

In addition, the conductometric PS sensor can be used as an array, where multiple conductometric PS sensors (e.g., one or more types of n-type conductometric PS sensors and one or more types of p-type conductometric PS sensors) are uniquely sensitive to different gases of interest thereby enabling an array to measure the concentration of multiple gases simultaneously (e.g., an appropriately treated conductometric PS sensor can be made to respond more strongly to one gas over a second gas). In addition, an array of conductometric PS sensors can be used to enhance sensing selectivity as the array of conductometric PS sensors provide multiple data points per tested sample which can be analyzed in a matrix format to provide selectivity for one gas over another based on the individual conductometric PS sensors within the conductometric PS sensor array. Thus, an array of conductometric PS sensors includes conductometric PS sensors sensitive to one gas over another and, in this sense, to select gases. In this regard, the array of conductometric PS sensors can be used to detect multiple analytes simultaneously.

### EXAMPLES

Now having described the embodiments of the present disclosure, in general, the following examples describe some additional embodiments of the present disclosure. While embodiments of the present disclosure are described in connection with the examples and the corresponding text and figures, there is no intent to limit embodiments of the present disclosure to this description. On the contrary, the intent is to cover all alternatives, modifications, and equivalents included within the spirit and scope of the embodiments of the present disclosure.

### Example 1

### Introduction

The incorporation of nitrogen can be used to vary the Lewis acidity of metal oxide metal sites. There are a significant number of approaches to accomplish this nitridation process on the oxides.¹⁻⁵ A well-established approach involves treatment with NH₃ at temperatures exceeding 500° C. for several hours.² This process has been applied to TiO₂ to produce the oxynitride, TiO₂₋ₓNₓ. More recently, in searching for microscopic strongly basic catalysts, Dogan et al.³ have created heavily nitrogen doped zeolites, treating these zeolites with NH₃ at temperatures in excess of 750° C. This process replaces the oxygen in both the Si—O—Si and Si—O—Al framework. These experiments lend credence to the use of amines in the nitridation process, as the Lewis acidity of the substituted metal oxide framework decreases due to the lower electronegativity of nitrogen with respect to oxygen. This is consistent with calculations obtained using density functional theory (DFT)⁴⁻⁶. An alternate approach takes advantage of the highly active nature of metal oxide nanoparticles. Porous anatase titanium dioxide nanocolloids in a size range 5-20 nm can be generated using sol-gel techniques⁷⁻⁹. These nanoparticles are found to be considerably more active than non-porous Degussa P25 TiO₂ nanoparticles, falling in the size range near 30 nm. They can be nitridated directly within seconds using alkyl ammonium compounds at room temperature to yield the visible light absorbing titanium oxynitride, TiO₂₋ₓNₓ. This chemical reaction produces significant heat as it forms the anatase oxynitride nanocolloid. By contrast, purified anatase Degussa TiO₂ requires several hours to produce an oxynitride which is not as effectively transformed and has not undergone the substantial changes observed for the nanocolloid. Further, any attempts to use the direct amination treatment at the microscale have been virtually unsuccessful, suggesting the important role played by the nanocolloid porous form and its interaction. The photocatalytic efficiency of the oxynitride formed from the nanocolloid also exceeds that of Degussa P25⁷⁻⁹.

The combination of tailored active interfaces, the ability to confine processes at the nanoscale, and the ability to manipulate nanostructured materials and their interaction at these select interfaces, offers the opportunity to develop economically viable, energy efficient, and sensitive sites for direct sensing and the heterogeneous base catalytic transformation of chemical species. The site preparation and interaction process driven largely by nanostructure-focused Lewis acid-base chemistry can provide array-based rapidly responsive and sensitive platforms¹⁰⁻¹⁶. In all cases we deal with Lewis acids and bases or Bronsted acids and bases. Here, we extrapolate on the nitridation of the titanium oxide nanocolloids and focus on the in-situ transformation of the metal oxides by reducing their Lewis acidity on substitution of nitrogen for oxygen, alternatively forming the more basic oxynitrides.⁷ Within this framework, the creation of highly active, nanopore coated, microporous, extrinsic semiconductor interfaces and their ability to be transformed at room temperature with selective nanostructure sites enables the efficient control of interface modification and electron transduction^{15,16}. Our overriding purpose in this study is to create and exemplify a means to broaden the response (matrix) of nanostructured metal oxide decorated interfaces with application to the complimentary inverse concepts of efficient chemical sensing and alternatively chemical conversion.

We have recently implemented the Inverse Hard/Soft Acid/Base IHSAB model¹⁰⁻¹⁹ as a means of linking chemical selectivity and the mechanism of sensor response. This model provides a simple-to-use prescription for design which relates, rationally, the physics and chemistry of specific nanostructure interfaces in microporous extrinsic semiconductor channels, combining the basic tenants of acid/base chemistry (the ability of bases to donate electrons and acids to seek electrons) and semiconductor physics. The mechanism of selectivity relies on the use of a nanopore coated microchannel array which combines optimized analyte diffusion with maximum interface interaction¹⁴. The nanoporous coating of the microchannel provides a unique phase match for the subsequent fractional deposition of select nanostructure islands that decorate the microchannel. The materials selected for the nanostructured islands serve the role of guiding gateways to force a dominant electron transduction (vs. chemisorption) at the decorated extrinsic semiconductor interface. The selection of these nanostructures and the variable and controllable physisorbed (reversible) interaction they introduce for sensor applications is well predicted by the IHSAB model¹⁰⁻¹⁹ as it dictates the coupling of analyte/interface acid-base interactions with the properties of the majority carriers in an extrinsic semiconductor. The inverse hard and soft acid and base (IHSAB) concept,¹⁰⁻¹⁹ complements the tenants of HSAB interactions.²⁰ Based on the reversible interaction of hard acids and bases with soft bases and acids, the IHSAB principle enables the selection of interacting materials that do not form strong covalent or ionic chemical bonds and thus it complements the HSAB model²⁰ for significant bond formation based on strong ionic (hard acid/base) or covalent (soft acid/base) interactions and chemical bond formation. The selection of the nanostructures that are deposited to the nanopore covered microchannels and the variable surface sensitivities that are produced, as they form metal/metal oxide deposits which can be transformed in-situ to the respective oxynitrides or oxysulfides, can be predicated based on a clearly designed procedure and established materials properties. The deposited metal oxide, oxynitride, and oxysulfide nanostructures are not formed via the application of time consuming self-assembly within the interface pores as the fractional deposition is far simpler to implement than traditional thin film or alternate coating techniques.

We emphasize that the nanostructures are deposited to form islands on the micropores of the extrinsic PS semiconductor. The nanostructures control and focus a variable and efficient analyte interaction and the transfer of electrons to or from the extrinsic semiconductor interface. The process can show a distinct time dependent dynamics as a function of nanostructure and analyte concentration.^{16,19,21} The in-situ transformation of the deposited nanostructured metal oxides to their corresponding oxynitrides facilitates a change in the interface response which we will demonstrate is explained within the IHSAB model and not simply based on an alteration of surface basicity. The formation of the oxynitrides or oxysulfides represents but one mode of functionalizing the decorated extrinsic semiconductor interface to expand the matrix of sensor responses. In complement, the extrinsic semiconductor is independently variable with a distinctly different band structure and electron dynamics associated with n or p-type doping.

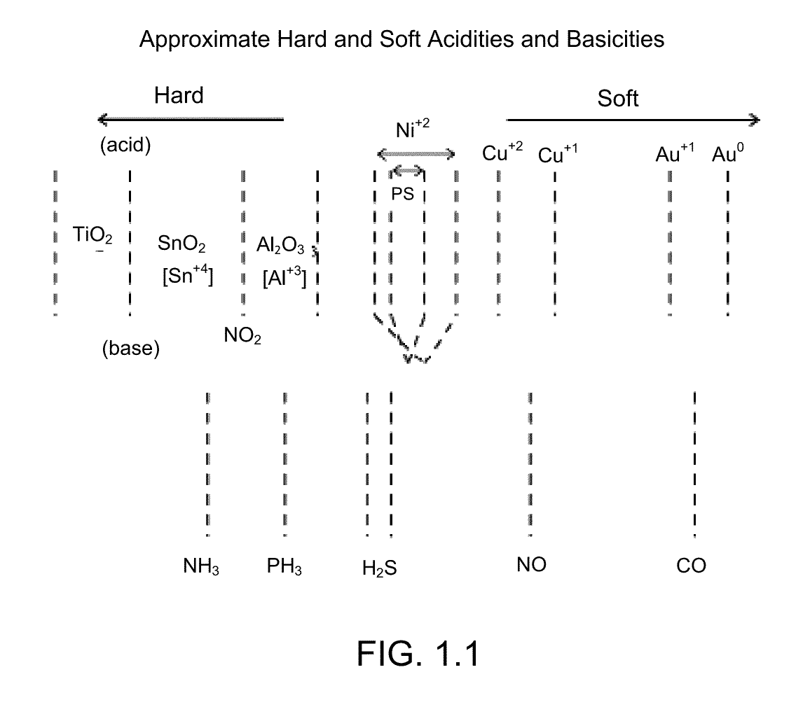

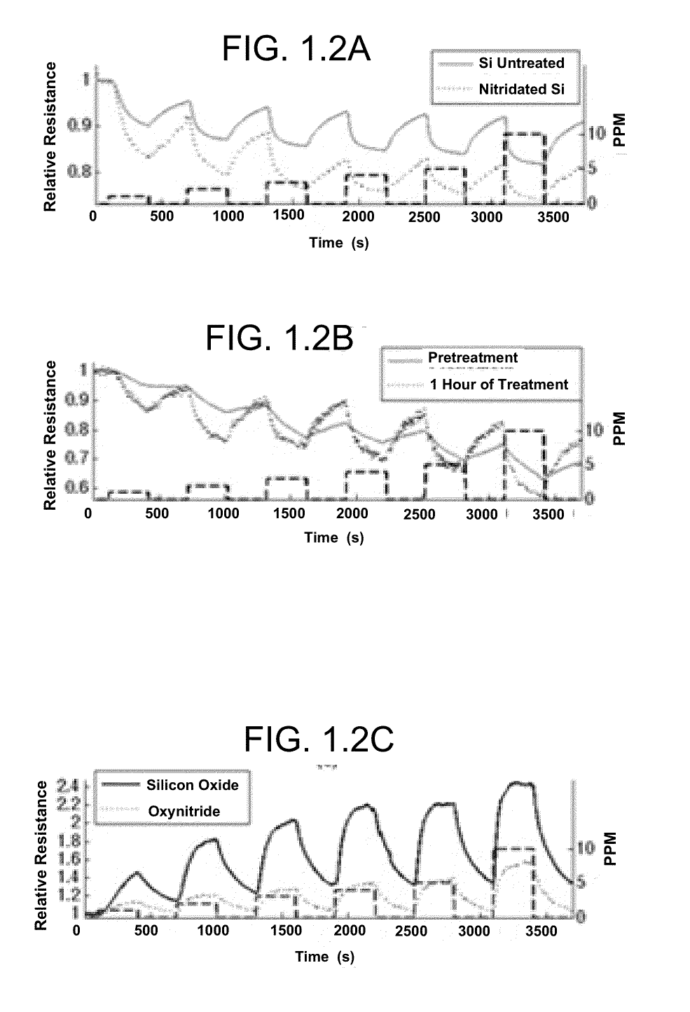

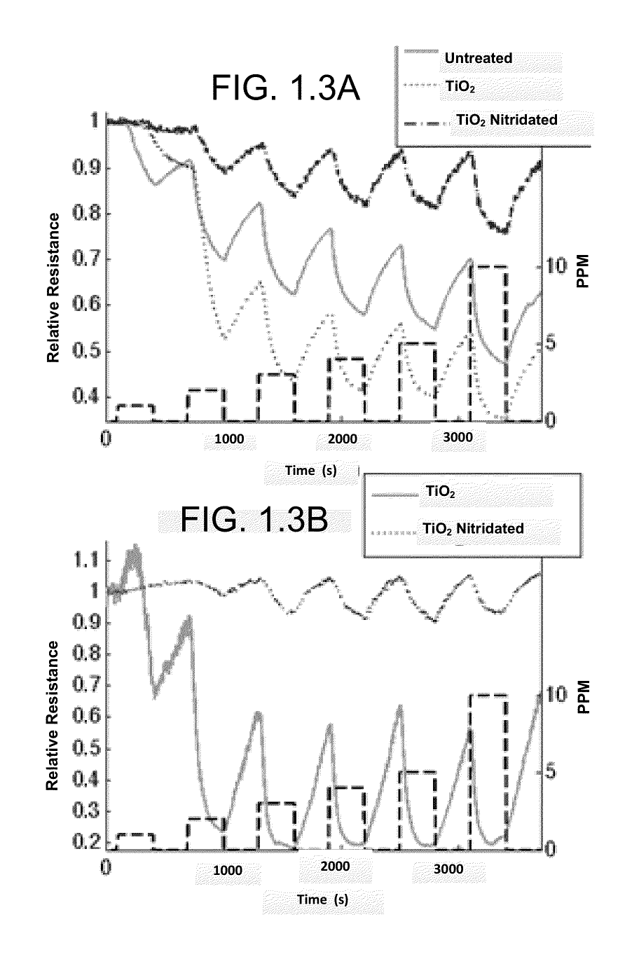

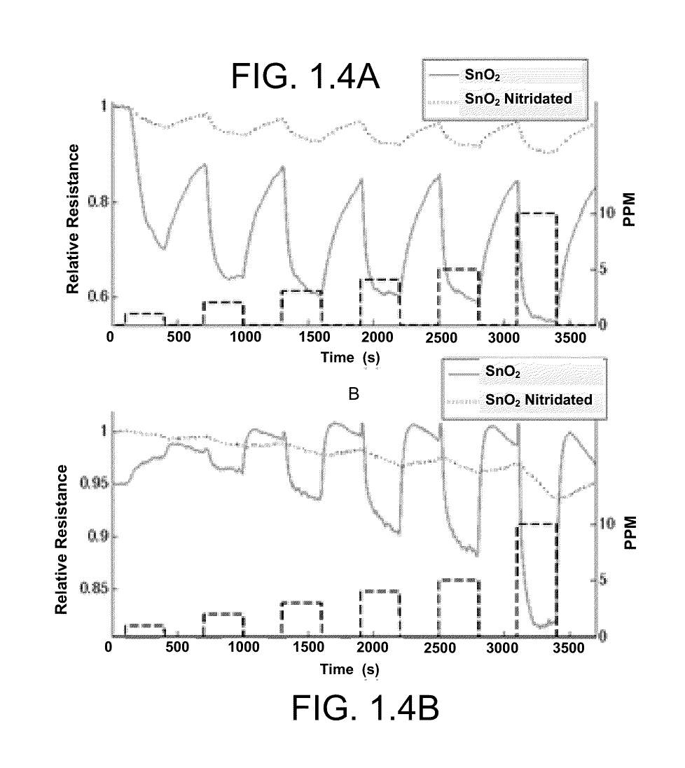

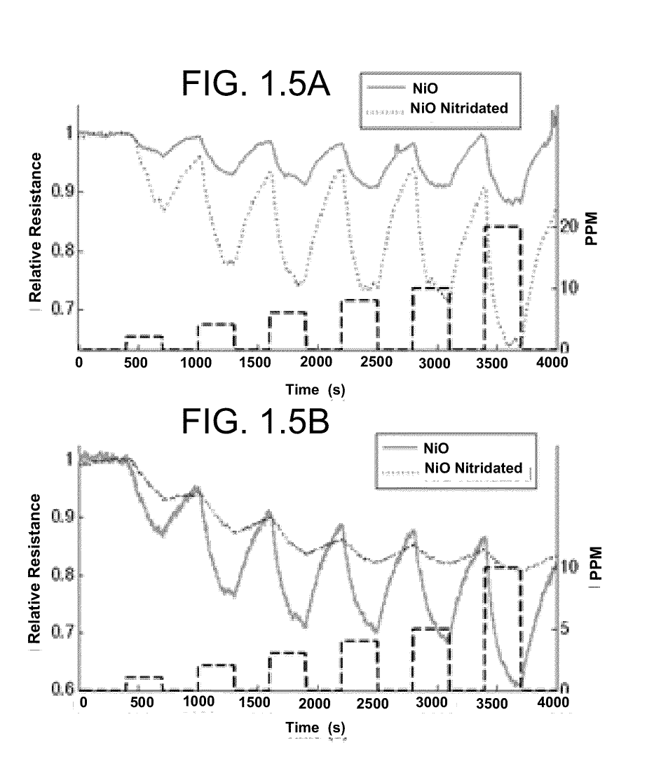

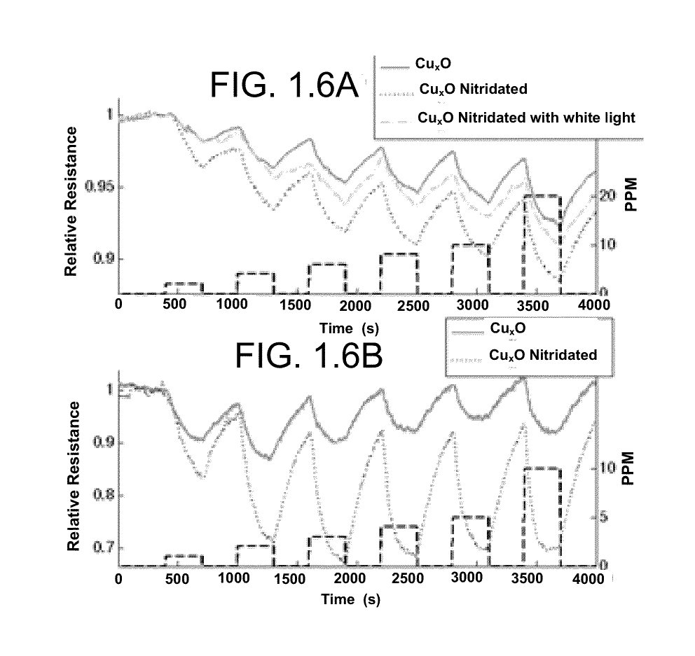

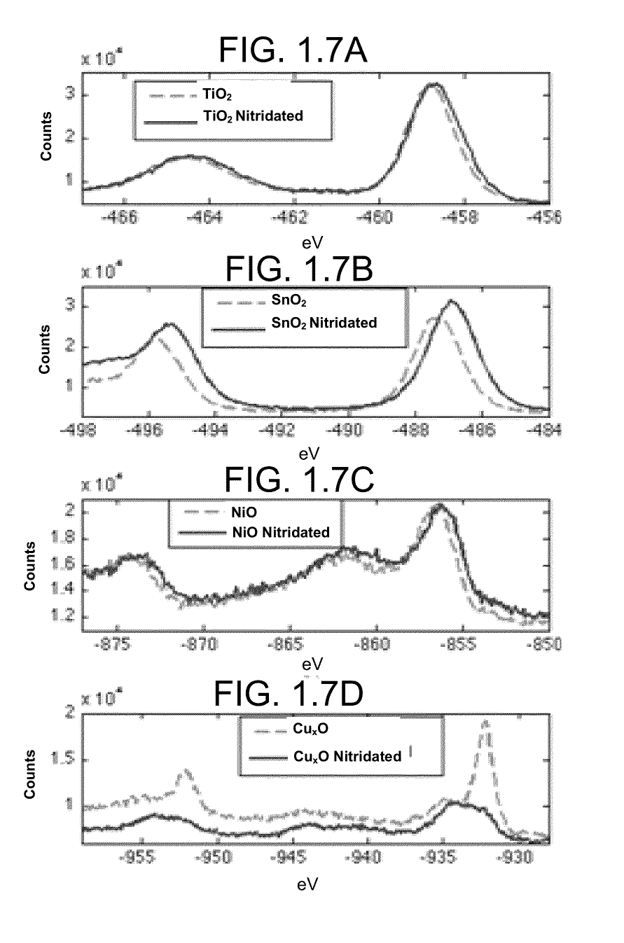

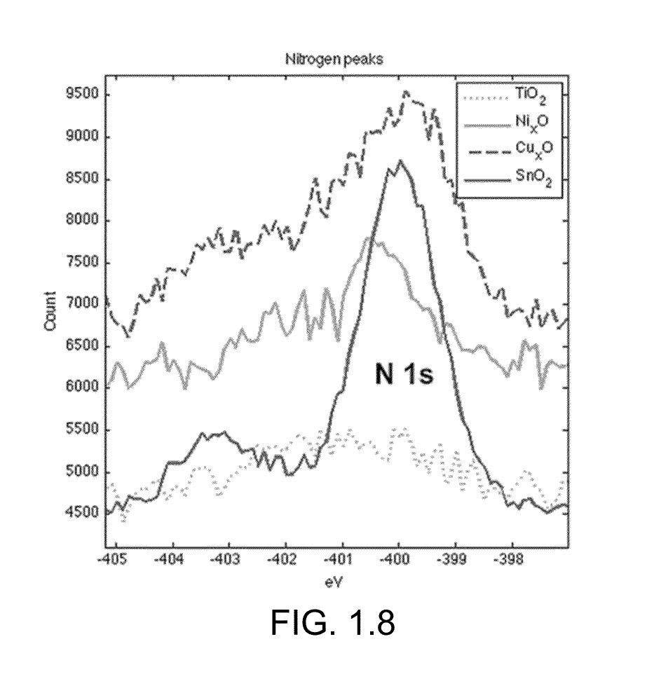

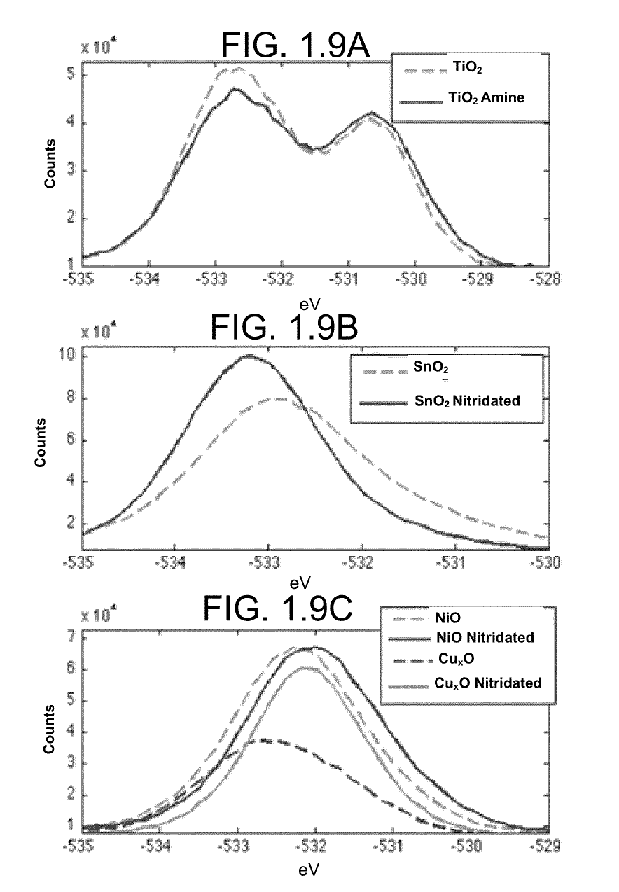

By focusing on the in-situ nitridation of metal oxide nanostructures deposited to an n-type extrinsic semiconductor interface, we therefore deal with analytes that modify the donor level electron population^{15,16,19}. A schematic model of the interface is shown in FIG. 1.10.

### Inverse Hard and Soft Acid/Base Concept and In-Situ Modification:

As an extrapolation of the HSAB concept developed by Pearson²² and later correlated within the context of density functional theory (DFT) by Pearson,²³ Parr, and others,^{23,24,25} the IHSAB model is somewhat broader-based and predicts reversible sensor-analyte interactions. Further details of this model are given elsewhere^{12,15} and in the Supporting Material. The relative responses given for n-type systems can be correlated to allow the construction of a “Materials Positioning Table” for the acids and bases within the IHSAB and HSAB concepts as summarized in FIG. 1.1. The analyte response data forms the basis for this development of the materials positioning diagram^{12,15} based largely on the interaction of the acidic metal oxides ranging from TiO₂ to AuₓO (x>>1) and the bases NH₃ to CO. The relative separation of the oxides and the bases within the range from hard to soft acids and bases dictates the observed responses of the interface. NH₃ displays a maximum reversible response for an AuₓO deposited surface whereas CO displays a maximum response for TiO₂ and SnO₂. Thus, in contrast to chemical bond formation, the greatest reversible response corresponds to the largest HOMO (highest occupied molecular orbital-donor)-LUMO (lowest occupied molecular orbital-acceptor) mismatch^{12,15}. The combination of responses for the analytes considered form the basis for selectivity, based on the combinatorial arrangement of arrays of decorated n and p-type PS interfaces^{15,19}, for which the interfacial structure of FIG. 1.1 can be generated. It is also feasible to expand the range of interface acidity by modifying the metal oxide nanostructure deposits, and we have obtained evidence for the facile in-situ transformation of the metal oxides to their corresponding oxynitrides and oxysulfides at the nanoscale.

In the following, we consider the nitridation of the porous silicon (PS) substrate and TiO₂, SnO₂, NiO, and CuₓO decorated PS. The interaction of these specific interfaces with NH₃ and NO has been studied.

In FIG. 1.1 the analyte scale is fixed in terms of acid/base properties as determined by the energy of the lone pair (lone electron) on the Lewis base donating to the positive Lewis acid metal site. The analyte lone pair energies can be evaluated from their ionization potentials or proton affinities (gas phase basicity) if the lone pair is the highest lying orbital. The orbital energies can be extracted from PES experiments if the lone pair is not the HOMO. The sensor scale in FIG. 1.1 can be varied by substituting N for oxygen, donating electron density into the metal. This lowers the Lewis acidity of the metal sites in the metal oxide. The apparent ability for in-situ transformation of the deposited metal oxide nanostructures can enhance the array of distinct responses which can be developed and extended to form “materials sensitivity matrices” for a given analyte as it provides a route to decrease the Lewis acidity of these acidic sites. Alternatively, the degree of nitridation can be used to introduce a progressively increasing site basicity at the nanoscale. This transformation is easily accomplished through direct amination in a manner analogous to that applied to the facile conversion of TiO₂ to TiO₂₋ₓNₓ^{7,8}. The in-situ formation of the oxynitrides shifts the transformed oxides toward the soft acid side of FIG. 1.1, adding breadth to this materials selectivity table and promoting a significant change in interface (sensor) response.

### Results and Discussion:

Nitridation Concept and Enhanced Basicity Associated with the Formation of Oxynitride Sites.

We consider the measurement of the in-situ change in response resulting from nitridation, as predicted by the IHSAB concept and its correlation with an enhanced basic character, gauged alternately by the softening of acidity (lowering of Lewis acidity) for the metal sites located within the deposited metal oxides. It is also possible to examine the surface chemistry of the nitridated nanostructures. By applying the decomposition reaction of methanol, it is possible to distinguish acid and base sites²⁶⁻²⁷ and concomitantly the transformation from acidic to basic sites. We exemplify this behavior by comparing the interactions of NO and NH₃ with a porous silicon and nitridated porous silicon interface and by studying the effect of nitridation on TiO₂, SnOₓ, NiO, and CuₓO nanostructure deposited PS interfaces¹⁵.

FIG. 1.2 compares the response for NH₃ and NO to untreated and nitridated n-type PS interfaces. FIG. 1.2A corresponds to the responses observed when NH₃ contributes electrons to an untreated¹⁵ and nitridated PS interface. Here, the PS interface is treated for 15 seconds with triethylamine. The interaction of NH₃ contributes electrons and increases the majority charge carrier concentration (electrons) and the conductance for both the nitridated and the untreated PS interfaces.¹⁵ However, the conductance increase (response) is considerably greater for the nitridated interface. This increase in response for the untreated PS interface is distinct from that of the TiO₂ and SnOₓ decorated PS interfaces. The response follows the IHSAB principle as the decorating metal oxides dominate the nature of the response (see following).

While the nitridation process enhances the basicity (decreases the Lewis acidity) of the PS surface corresponding to a shift toward the soft acid side of FIG. 1.1, the HOMO (donor)-LUMO (accepter) orbital mismatch increases and the IHSAB principle predicts an increased response after nitridation. FIG. 1.2B demonstrates that the nitridation process extended to one hour produces a similar, if not more pronounced, modification of the PS interface response. We observe a more rapid rise in system response as well as signal decay as the gas flow is removed. The untreated PS interface appears to display a t^{1/2} dependence. The interface after nitridation appears to display a more exponential response indicative of a degree of pore expansion upon nitridation. FIG. 1.2C corresponds to the response observed when NO interacts with an n-type PS interface. Here, with the untreated PS interface, the amphoteric NO radical acts as a weak acid^{15,19}, withdrawing electrons and increasing resistance (decreasing conductance). This is a complex process. However, nitridation of the PS interface inhibits the extraction of electrons by NO and leads to a decrease in the resistance response. The decrease in response for the untreated PS interface is distinct from that of NH₃ and the NiO and CuₓO decorated PS interfaces as the response follows the IHSAB principle and the decorating metal oxides dominate the nature of the response (see following). The nitridation of the PS interface decreases the HOMO-LUMO orbital mismatch. The data in FIGS. 1.2A-C, describing the PS interface response, provides a backdrop for the consideration of those changes which accompany the nitridation of the metal oxide nanostructure deposited surfaces we consider.

FIG. 1.3 compares the response for NH₃ and NO to a TiO₂ nanostructure deposited n-type PS interface, before and after nitridation. Our recent studies of visible light absorbing TiO₂₋ₓNₓ photocatalyst nanoparticles^{7,8} suggest the direct nitridation of highly porous TiO₂ nanocolloids.

We find that in-situ nitridation modifies the response of the semiconductor interface which has been deposited with TiO₂ nanoparticles. Within the IHSAB format^{12,14,15,17-19,}, the effect can be considered by recognizing that nanostructured TiO₂ represents a strong (hard) acid. Its oxynitride, TiO₂₋ₓNₓ, once formed, through in-situ treatment of the TiO₂ deposited surface, has gained considerable basic character. Alternatively, the Lewis acidity of the titanium center has decreased due to the smaller electronegativity of nitrogen which reduces the ionicity of the M—N bond and makes M less acidic. Here the electronegativity of N is in contrast to the formal charge distribution which makes it N³⁻. It is to be noted that if N goes into the oxide lattice as N²⁻ there is an extra electron present that can modify the acid/base character.

The data in FIG. 1.3A compares the response of an untreated n-type PS interface, upon exposure to 1-5 and 10 ppm of NH₃, for the PS interface deposited with acidic TiO₂ nanostructures, and this same interface where the deposited nanostructures are converted in-situ from TiO₂ to the more basic TiO₂₋ₓNₓ^{7,8}. Here, the TiO₂ concentration may be somewhat less than that necessary to produce an optimal response¹⁶. TiO₂ with Ti as a strong Lewis acid, enhances the capture of electrons, transferring these electrons to increase conductance (decrease resistance) relative to the undecorated interface. The formation of the oxynitride decreases the metal site Lewis acidity and does not facilitate electron transduction as efficiently. The sensor response corresponds to a conductance decrease relative to the TiO₂ treated interface. The in situ nitridation of TiO₂ shifts the nature of this metal oxide nanostructure toward the soft acid side of FIG. 1.1, closer to ammonia. The IHSAB principle dictates that the response of the TiO₂₋ₓNₓ interface should decrease relative to TiO₂ as it indeed does. However, the nitridation process does not simply increase the basicity of the nanostructured interface. A weak interaction with minimal chemical bonding occurs if the donor orbital (highest occupied molecular orbital, HOMO) energy is not well matched with the acceptor (lowest occupied molecular orbital, LUMO) energy. The nitridation affects the LUMO energy as it is raised by decreasing the Lewis acidity. The HOMO of the molecule to be sensed is not changed and the HOMO (donor)-LUMO (acceptor) energy gap decreases. This produces more charge transfer and a stronger Lewis acid-base interaction. The orbital matchup and Lewis acid-base bonding with NH₃ is enhanced, leading to increased chemisorption and decreased sensor response. By comparison, the sensor response of the TiO₂₋ₓNₓ interface decreases relative to TiO₂. In both concert, and contrast to the behavior expected for a simple increase in interface basisity, the sensitivity of the weaker metal oxides can be enhanced by nitridation.

FIG. 1.3B corresponds to the response observed when NO interacts with a TiO₂ treated n-type PS interface. TiO₂, as a strong acid, completely overcomes electron withdrawal by NO (FIG. 1.2C) as the TiO₂ decorated PS interface extracts electrons resulting in a significant increase in conductance (drop in resistance). This electron extraction also should form the more strongly bonded NO⁺. The TiO₂ concentration can be optimized further to produce a greater drop in resistance¹⁵. Nitridation of the TiO₂ decorated PS interface inhibits this electron extraction and leads to a considerable decrease in conductance.

FIG. 1.4 compares the responses to NH₃ and NO for an SnOₓ nanostructure deposited n-type PS interface, before and after nitridation. Nanostructured SnOₓ, especially as SnO₂, corresponds to a strong acid, however, its acid strength is notably less than that of TiO₂. Hence its positioning in FIG. 1.1. The oxynitride, “SnO₂₋ₓNₓ”, once formed, through in-situ treatment of the SnO₂ deposited interface shows a decreased Lewis acidity and thus gains considerable basic character. FIG. 1.4A corresponds to the responses observed when NH₃ contributes electrons to an interface deposited with SnO₂^{12,15,19} and subsequently nitridated. We compare the response of an untreated n-type PS interface, upon exposure to 1-5 and 10 ppm of NH₃ for the interface treated with a deposition of acidic SnO₂ nanostructures, and this same interface where the deposited nanostructures are converted in-situ from SnO₂ to the more basic SnO₂₋ₓNₓ. FIG. 1.4B corresponds to the responses observed when NO interacts with an SnO₂ treated n-type PS interface.¹⁵

FIG. 1.4B corresponds to the responses observed when NO interacts with an SnO₂ treated n-type PS interface.¹⁵ The strong acid SnO₂ decorated interface again overcomes electron withdrawal by NO (FIG. 1.2C), although not to the extent of the TiO₂ decorated PS interface. Electrons are extracted from the NO, resulting in a significant increase in conductance (drop in resistance). Nitridation of the SnO₂ decorated PS interface again inhibits the electron extraction as nitrogen replaces oxygen, lowers the Lewis acidity of the tin centers, and leads to a considerable decrease in conductance and hence response.

FIG. 1.5 compares the response for NH₃ and NO to an NiO nanostructure deposited n-type PS interface, before and after nitridation. Nanostructured NiO corresponds to a moderate acid, hence, the positioning of NiO in FIG. 1.1. Its oxynitride, “NiO₁₋ₓNₓ”, once formed, through in-situ treatment of the NiO deposited interface gains considerable basic character as the Lewis acidity of the nickel site decreases.

FIG. 1.5A corresponds to the responses observed when NH₃ contributes electrons to an interface treated with NiO, (1) formed from oxidation after electroless Ni deposition and, (2) subsequently nitridated. We compare the response of an untreated n-type PS interface, upon exposure to 1-5 and 10 ppm of NH₃ to the interface treated with a deposition of NiO nanostructures, and this same interface where the deposited nanostructures are converted in-situ from NiO to the more basic “NiO₁₋ₓNₓ”. Here, after nitridation, the response to ammonia increases. This surprising response can be explained within the framework of the IHSAB principle.

FIG. 1.5B corresponds to the responses observed when NO interacts with an NiO treated n-type PS interface.¹⁵ NiO and NO compete for the available electrons in this system, especially at higher NO concentrations.¹⁵ The process is dynamic and, under the conditions of the present experiment NiO dominates. Nitridation of the NiO decorated PS interface again inhibits electron extraction by the decorated interface and leads to a considerable decrease in conductance. The observed processes associated with nitridation in FIGS. 1.5A and 1.5B taken together are consistent with (1) a greater HOMO (donor)-LUMO (acceptor) energy mismatch in energy for NH₃ and (2) a decrease in the HOMO-LUMO energy mismatch for NO.

FIGS. 1.6A and B compare the response to NH₃ and NO of a CuₓO nanostructure deposited n-type PS interface, before and after nitridation. Nanostructured CuₓO corresponds to a moderate to weak acid, hence its positioning in FIG. 1.1. Its oxynitride, “CuₓO_{1-y}N_{y}”, once formed, through in-situ treatment of the CuₓO deposited interface gains considerable basic character.

The data in FIG. 1.6A compare the response upon exposure to 1-5 and 10 ppm of NH₃ for a PS interface treated with a deposition of CuₓO nanostructures, and this same interface where the deposited nanostructures are converted in-situ from CuₓO to the more basic “CuₓO_{1-y}N_{y}”. Here, after nitridation, the response to ammonia increases! This surprising response can be explained within the framework of the IHSAB principle.

FIG. 1.6B corresponds to the responses observed when NO interacts with a CuₓO treated n-type PS interface.¹⁵ CuₓO and NO again compete for the available electrons in this system¹⁵ and, under the conditions of the present experiment CuₓO dominates. Nitridation of the CuₓO decorated PS interface now increases the electron extraction and leads to a considerable increase in conductance! This is not consistent with a simple increase in the basic character of the interface but is consistent with the IHSAB principle.

Response Matrices and their Modification on Nitridation:

Table 2 summarizes the effects of nitridation on TiO₂SnOₓ, NiO, and CuₓO decorated PS interfaces. Here, we summarize whether nitridation produces a weaker or stronger response than that of the untreated metal oxide. The data in Table 2point to an affect which results from a more complex interaction than simply converting the metal oxides to weaker Lewis acids (increasing basicity).

The interaction of a given analyte with the four acids we have studied is summarized by a group of responses which are distinctly different than those of an untreated PS interface. The relative responses of the decorated metal oxide interfaces and there corresponding nitridated counterparts are summarized in Table 3. The resistance change per base resistance for the nanostructure deposited vs. untreated PS interfaces is given by Equation 1. Since there can be OH groups and hydrocarbons, originating from the air, deposited to the interface, we treat all of the systems studied here with UHP N₂ before use to insure a significant flushing of water from the surface. In addition we perform relative measurements with the identical previously characterized untreated sensor when evaluating the changes resulting as nanoparticles are deposited to the PS interface. An N₂ flow onto the sensor is kept constant at 200 sccm at all times during the experiment. Diluted NH₃ and NO are mixed (separately) with the N₂ flow as we test the system response for reversible sensitivity.

TABLE 2
Summary of relative oxynitride treated vs. oxide responses for the
interactions of NO and NH₃ with porous silicon (PS), TiO₂ treated PS,
SnOₓ treated PS, NiO treated PS, and CuOₓ treated PS.

| | NO | NH₃ |
|---|---|---|
| PS nitrated 15 seconds | weaker | stronger |
| PS nitration 1 hour | — | very strong |
| TiO₂ | weaker | weaker |
| SnOₓ | weaker | weaker |
| NiO | weaker | stronger |
| CuOₓ | stronger | stronger |

$$Δ = \frac{Δ R ( deposited ) \text{/} {R}_{0} ( deposited )}{Δ R ( untreated ) \text{/} {R}_{0} ( untreated )} \qquad ( 1 )$$

The data in Tables 3 and 4 demonstrate that the response matrices for the metal oxides and there nitridated counterparts are distinct and that the nitridation process creates additional distinct response matrix elements that can be used to test for a given analyte. These response matrix elements are determined by the acid/base strength of the analyte relative to that of the nanostructure deposited interface. This is exemplified in FIG. 1.1.

TABLE 3
Relative responses for nitridated metal oxides in comparison to
nanostructured metal oxides for conductometric responses to NO and NH₃.

| | NO | NH₃ |
|---|---|---|
| TiO₂ | 0.025 | 0.11 |
| SnOₓ | .28 | .14 |
| NiO | .5 | 3 |
| CuOₓ | 2 | 1.5 |

The dependence is not a simple one but is dictated by the manner in which the nitridation process modifies the molecular orbital makeup of the metal oxides as the nitration process adjusts the HOMO (donor)-LUMO (acceptor) energy separation and enhances or diminishes the molecular orbital matchup of the analytes and decorating nanostructures. The method of producing individual reversible sensors, could be employed to produce combinations of array based multiple sensor devices of varying sensitivity to a variety of gases and a matrix of array responses might be correlated to selectivity for a given gas mixture.

TABLE 4
Relative responses of nitridated metal oxides vs. untreated porous
silicon (PS) for conductometric responses to NO and NH₃.

| | NO | NH₃ |
|---|---|---|
| PS nitrated 15 seconds | .5 | 2 |
| PS hour nitration | — | 2.2 |
| TiO2 | −.25⁸ | .25 |
| SnOx | −.5⁸ | .33 |
| NiO | 2 | 4.5 |
| CuO | 2.4 | 4 |

* The negative numbers correspond to the reversal of the signal for the untreated porous silicon sensor (please see Ref. 15).

X-Ray Photoelectron Spectroscopy (XPS)

XPS profiles for the TiO₂, SnOₓ, NiO, CuₓO and their corresponding nitridated counterparts are show in FIGS. 1.7-1.9. Here, we monitor the electron binding energy of sites a few nanometers from the decorated interface surfaces. We have examined six areas of the XPS spectrum, the Ti 2p, Sn 3d, Ni 2p, and Cu 2p, regions, the N 1 s region near 400 eV, and the O 1 s region near 530 eV.

In scanning the Ti 2p regions as depicted in FIG. 1.7A we find a slight shift of the Ti binding energy from 458.8 eV, decreasing to 458.6 eV upon nitrogen incorporation, a shift of approximately 0.2 eV to lower binding energy. The Sn 3d binding energy associated with SnOₓ (FIG. 1.7B) shifts by 0.3 eV to lower binding energy. The Ni 2p binding energy associated with NiO (FIG. 1.7C) is at ˜853.7 eV and shows a slight red shift of approximately 0.2 eV upon nitridation.

In contrast to the XPS spectra for TiO₂, SnOₓ, and NiO, the Cu 2p peaks for the nitridated CuₓO sample show not only a weakening and broadening of the XPS features but also a shift to higher binding energy. The N 1s XPS features associated with the nitridation process are depicted in FIG. 8 whereas the corresponding O 1s XPS spectra are depicted in FIG. 1.9.

The XPS spectra obtained in FIGS. 1.7-1.9 are sensitive to 0.1%. The typical time frame for the depositions used to obtain the conductometric data in FIGS. 1.2-1.6 is 30 seconds. The XPS data requires that we use deposition times which are at least 5 minutes. This corresponds approximately to an order of magnitude increase in concentration to obtain signals in the 0.1% range.

The N 1s XPS binding energies observed for the N-doped oxides (FIG. 1.8) are broad and extend from ˜398 to 403 eV with the exception of SnO₂ which extends to 401 eV. In all cases, this range exceeds the binding energy of 397.2 eV in TiN³⁰. The shift in the N is features to higher binding energy results when the nitrogen is more positive. It is 408 eV in NaNO₃ (formal +5 on N) compared to 398.8 eV³¹ in NH₃ where the nitrogen has a negative formal charge of −3. Rodriguez et. al^{32,33} have carried out an XPS analysis of the interaction of NO₂ with several polycrystalline surfaces, observing a strong peak at 404.5 eV, intermediate to that for absorbed NO (400-401.5 eV^{33,34}) and NO₃,³⁵ which they tentatively assign to NO₂.

They also observe weak features at 396.5 eV and 401 eV which they assign to N and NO in a metal lattice. The observed features, which we observe are consistent with the N is features for NO. They increase in intensity from the TiO₂ treated to the CuₓO treated interface. The observed XPS peaks for the Ti 2p region and their change with nitrogen incorporation are quite consistent with an XPS depth profiling characterization carried out by Gyorgy et. al.³⁶ These authors have also observed a shift in the Ti 2p binding energy to lower energies as the TiO₂ surface is nitrided. Further, the observations in the present study are consistent with the data obtained by Chen et al.³⁷ on TiO₂₋ₓNₓ and the earlier results of Saha and Tomkins²⁹ who have used XPS to characterize the oxidation of a titanium nitride surface.

The observed binding energies for the O 1s XPS spectra are given in FIGS. 1.9A, 1.9B, and 1.9C. They show significant changes upon nitridation of the metal oxide. The data for TiO₂ are quite interesting verses the typical data that we have observed³⁷ previously for TiO₂ and its nitridated counterpart TiO₂₋ₓNₓ. The double peak in FIG. 1.9A is, in fact, due to both SiO₂ (high energy peak at 532.3 eV) and TiO₂ (low energy peak at 530.2 eV) as the signals for these oxides overlap each other.

The data indicate that the nitridated form of both TiO₂ and SiO₂ show a slight increase in binding energy to 532.4 and 530.3 eV respectively. The SnOₓ decorated systems depicted in FIG. 1.9B indicate a significant decrease in binding energy from 532.8 eV to 531.9 eV with nitridation. The NiO decorated interface shows a significant decrease of ˜1.8 eV in the O 1 s binding energy (FIG. 1.9C) on nitridation and the decrease in binding energy for the CuₓO decorated interface is even greater (˜2.8 eV). These are clear trends and distinctions.

### Discussion:

The responses reported in FIGS. 1.2-1.6 can be explained within the concept of the IHSAB principle. The underlying IHSAB principle dictates the physisorption (electron transduction) directed response to a number of basic, acidic, and amphoteric analytes as they interact with nanostructure decorated p and n-type PS extrinsic semiconductor interfaces^{12,15,19}. A first order comparison of response data with the exemplary list of hard, borderline, and soft acids and bases demonstrates that hard bases such as NH₃ respond most strongly when exposed to an AuₓO nanostructure treated PS surface^{10,15,19}. Within the framework of the IHSAB principle, AuₓO is a soft acid (Au^{0,+1}) and should provide a strong response to NH₃^{11,12,17}. The results which we obtain for the amphoteric NO radical demonstrate the importance of the relative acidity of the nanostructured metal oxide deposits which are placed on the PS interface. This is more strongly manifest on an n-type as opposed to a p-type PS interface^{15,19}. The ability and efficiency of these nanostructured sites to direct electron flow dictates whether NO acts as either a weak base or a weak acid. The responses to NO for an n-type PS interface^{15,19} also can be correlated to demonstrate consistency with the materials positioning diagram of FIG. 1.1. By monitoring the trends in hard and soft acid and base behavior, first order selections can be made for the modification of the PS hybrid interface (or any extrinsic semiconductor interface onto which a nanopore coated microporous structure can be formed) with nanostructured metal oxide deposits to create a range of selectivities (responses) for a number of gases. With this data, continued improvements and extensions of FIG. 1.1 can be generated. The data that we have presented suggest that we can modify the acid/base properties of the metal oxide (metal centers) outlined in FIG. 1.1 and the sensor scale by varying the metal positive charge through doping in-situ with nitrogen substituted from appropriate precursors. Here, we lower the Lewis acidity of the metal oxide (metal) sites. This will shift the metal toward the softer acid side in the top portion of FIG. 1.1. The bonding interaction with the fixed analytes catalogued in the bottom of this figure will increase or decrease in complement, and the sensor signal will decrease or increase. The change in Lewis acidity does not shift the doped TiO₂ further to the right than the fixed position of NH₃ so the signal response for both NH₃ and NO will decrease and remain of the same sign. However for the interaction with a metal site between two analytes, for example, Ni²⁺ the situation is modified. Ni²⁺ is approximately equidistant between NH₃ and NO. As its Lewis acidity decreases, the signal from NO will decrease and that for NH₃ will increase. These patterns provide a basis for increasing the breadth of the sensitivity matrices and modifying the relative responses of the interface sites thus creating Tables 3 and 4 in a well prescribed manner.

There are different ways to control the size of the interaction of those molecules that are to be sensed with variably doped metal oxide nanostructure deposited sensing interfaces. If the orbital orientation at the surface is not correctly configured, there can be little binding with the lone pairs of the incoming molecules. In addition, a combination of molecular and surface steric effects could also block the interaction at the surface by preventing orbital overlap. However, the dominant factor is the HOMO-LUMO energy increment. If the donor orbital energy (highest occupied molecular orbital, HOMO) is not well matched with the acceptor (lowest unoccupied molecular orbital, LUMO), then the interaction will be weak. As the HOMO (donor)-LUMO (acceptor) energy gap decreases, there can be more charge transfer between the molecule and the sensor interface, leading to a stronger Lewis acid-base interaction. For Lewis acid-base bonds, the donor retains the electron pair, a prototypical example being BH₃NH₃, with a B—N bond dissociation energy (BDE) of ˜26 kcal/mol²⁸. At the other extreme is the interaction of an anion and a cation forming an ionic bond with a much larger BDE. If the electrons are fully shared leading to the formation of a covalent bond this can also lead to a large BDE. The IHSAB is in large part based on controlling the size of the Lewis acid-base bond dissociation energy.

The results obtained for conductometric response display a clear quantitative dependence on concentration, however, they are based on semi-quantitative inferences from measuring the sensing signals from the interactions of molecules interacting with surfaces via donor-acceptor interaction. More detailed physical measurements on the structures of the surfaces and the energetics of these surfaces will provide enhanced understanding. Molecular data needed to address the orbital energy arguments is available in terms of molecular proton affinities, acidities, and ionization potentials but these data are not broadly available for surfaces²⁹ or nanoclusters. While our measurements now provide semi-quantitative data about the doped metal oxide surface sites, further experiments will help to quantify that data.

The XPS data obtained for TiO₂, SnO₂, NiO, and CuₓO and their oxynitrides would seem to correlate with the data in FIGS. 1.2-1.6. In particular, we note that the conductometric data in FIGS. 1.6A and B which demonstrate a significant increase in the signal for the nitridated sample are consistant with the 1) the decrease in signal intensity and 2) the increase in nitrogen binding energy (FIG. 1.7D) observed for the copper system. The clear trend in the decrease of the O 1s binding energy from the titanium to copper oxides upon nitridation is consistent with the decrease in the titanium, tin, and nickel site binding energies. However, the data in FIG. 1.7D) are consistent with an increased stability for the nitridated copper sites. The copper (FIG. 1.7D) and oxygen (FIG. 1.9C) data, in concert, would suggest a more significant rearrangement of the nitridated lattice for the copper system.

The process of physisorption/electron transduction must involve the interaction of high-lying occupied or low lying unoccupied molecular orbitals of each individual gas with the decorated PS interface. If these gases, as bases, are the electron donors (acceptors-acid) then the surface, as an acid, is an electron acceptor (donor-base), represented by the primarily acidic metal oxides used to modify an n or p-type PS interface. This process will differ from gas to gas and with changes in the nanostructured deposit. However, the nature of the interaction, as it provides for increased electron transduction and minimizes chemical bond formation (chemisorption), thereby influencing the efficient flow of electrons to or from a gaseous analyte as it interacts with the decorated PS interface, provides the dominating basis for the observed resistance (conductance) changes seen in FIGS. 1.2 to 1.6. The presence of a fractional nanostructured oxide deposit on the PS surface promotes further interaction with the interface and is distinct for each metal oxide. The process whereby a gas transfers or withdraws electrons as it interacts with that surface will be strongly influenced by the balance of chemical bonding, which greatly inhibits electron flow, and physical absorption, which can be made to facilitate electron flow.

The precise details of the mechanism for the resistance change which appears to be characteristic of virtually all oxidizing (increased resistance) and reducing (decrease in resistance), gases on the individually modified hybrid n-type PS interfaces and their counterparts in a p-type system will require further experimentation and modeling. However, if we consider an appropriate sensor mechanism involving interaction with the metal oxide surface subgroups, the fractional nanostructure deposition of an n-type sensor is consistent with the changes in resistance that we outline above. Basic analytes will attempt to provide an electron to the PS interface whereas acidic analytes will attempt to remove an electron, leading to an increase or decrease in the number of majority charge carriers. This process will be influenced by the nature of the decorated PS interface. The key issue is the orbital makeup of the analyte with that of the decorated interface. As we modify these molecular orbital makeups, we change the relative positions and matchups of the analyte and interface.^{12,15,19} This is the essential proposition for creating the arrangement of the data outlined in FIG. 1.1.

The in-situ nitridation of TiO₂ shifts the nature of this metal oxide nanostructure toward the soft acid side of FIG. 1.1 and as a result of the molecular orbital makeup of the oxynitride, it lies closer to ammonia. The IHSAB principle dictates^{12,14,15,17-19,26} that the orbital matchup with and chemical interaction with NH₃ is enhanced and therefore the response of the TiO₂₋ₓNₓ interface should decrease relative to TiO₂ as it does. Similar decreases in the observed sensor response are observed as nitridated SnO₂ interacts with NH₃ and NO with which its molecular orbital makeup (LUMO) is now more closely aligned with the HOMO of NH₃ and NO. The nitridation of NiO leads to a decrease in response for NO, however, the reversible response for interaction with NH₃ increases. This may seem surprising, yet it is completely consistent with the IHSAB concept. Nitridation shifts the molecular orbital makeup of NiO closer to NO but leads to a greater HOMO (donor)-LUMO (acceptor) orbital mismatch with NH₃. The nitridation of CuₓO again decreases the metal site Lewis acidity (forms more basic sites) and shifts the response of the modified nanostructures further to the soft acid side of FIG. 1.1. The behavior of the nitridated CuₓO again may seem surprising. It is tempting to suggest that the formation of the oxynitride should simply increase the basicity of the nanostructure surface and thus should decrease the response to NH₃ However, this does not occur. The nitridated copper oxide is shifted further to the soft acid side of ammonia in FIG. 1.1, dictating a greater HOMO (donor)-LUMO (acceptor) molecular orbital mismatch and an enhanced reversible response to ammonia. The IHSAB principle suggests, counter to intuition that the response of the in-situ treated nitridated copper oxide interface should increase relative to that of CuₓO, precisely as is observed. In FIG. 1.1, NO is positioned directly under the copper oxides. Nitridation shifts the copper oxides to the soft acid side and away from NO, again leading to an increase in molecular orbital mismatch and an increase in the reversible response of the oxynitride to NO. Finally, the behavior observed for the untreated and nitridated PS interfaces is consistent with the IHSAB principle. FIG. 1.1 can be used to demonstrate that the nitridation of the PS interface causes a shift away from NH₃, which leads to an increase in the reversible response to NH₃ and a shift toward NO which leads to a decrease in the reversible response to NO. These results which we have obtained with nitridation suggest that the IHSAB principle can be used as an important distinguishing principle of reversible sensor response as dictated by the dominance of electron transduction over bond formation.

### Conclusion:

We have demonstrated the efficacy of fractional nanostructure depositions as a means of obtaining distinct reversible interface responses which show the potential for combination in an array based format. We have considered the conversion of the nanostructured metal oxides in-situ to their oxynitrides and the enhanced basicity that this introduces to a nanostructure decorated PS interface. The behavior of these systems appears to be well represented by the newly developing IHSAB model^{12,15,19}. These systems also display a time-dependent dynamics which must be incorporated into the IHSAB model^{16,19}. This will be the subject of future studies.

### Experimental:

Virtually all of the experimental procedures with the exception of nitridation have been discussed previously^{12,14,16,19}. A hybrid etch procedure is used to generate nanopore covered micropores. Schematic diagrams of the complete working sensor platform have already been presented^{12,38,39}. The PS interface is generated by electrochemical anodization of 1-20 ohm-cm, n-type (phosphorous doped) (100) silicon wafers. The anodization of the n-type wafers^{40,41} is done under topside illumination using a Blak-Ray mercury lamp. The silicon wafer is etched in a 1:1 solution of HF and ethanol at a current between 8-15 mA/cm^{2 8,39-41}. The anodized n-type sample is placed in methanol for a short period and subsequently transferred to a dilute HF solution for a 30 minute period. This process creates a porous structure with pore diameters of order 0.5-0.7 μm and pore depths varying from 50 to 75 μm.

The PS hybrid arrays of nanopore covered micropores are tested at room temperature for their individual interface response. The nature of this response is evaluated on the basis of the IHSAB acid/base principle. The selection of the nanostructures and the variable surface sensitivities that are produced as they form in-situ metal oxide deposits, introduces a distinct systematics of design. The approach is unique in that the nanostructures are deposited fractionally to the PS micropores and this fractional deposition DOES NOT require any time consuming self-assembly within the pores. This is not a coating technique or one that requires an exacting structural film arrangement but is, in fact, a much simpler process. The nanostructure deposition must be maintained at a sufficiently low level to avoid cross-talk between the nanostructures that, as it increases, leads to a noisy device and the eventual loss of functionality. In combination, these can be used as a basis to develop selectivity. Results obtained with nanostructured deposits generated from electroless tin, nickel, and copper, as well as nano-titania are considered in this study. All of the nanostructured metals deposited to the PS surface are readily oxidized to SnOₓ (x=2, 4), NiO, and CuₓO (x=1, 2) as demonstrated by XPS measurements³⁹. This in-situ formation provides metal oxide decorated interfaces which appear to be more sensitive than those previously obtained.^{12,15,19}

The initially introduced titania (anatase) may be crystalline, however, we cannot be certain of this crystallinity after deposition to the PS interface. The untreated PS hybrid structures are exposed to the electroless solutions for 10 to 30 seconds and are placed in DI H₂O and MeOH for consecutive 120 second periods. The oxidized electroless metal depositions when characterized before deposition correspond to amorphous structures displaying no diffraction patterns. Therefore, it is difficult to envision their crystallization during the short deposition and subsequent surface cleaning process. Basic character (a lowering of Lewis acidity) is introduced to the nanostructured metal oxides by direct in-situ treatment with triethylamine (TEA). The metal oxide treated surface is exposed to the TEA for 10-30 seconds. The treated interface is subsequently washed in methanol to remove excess TEA and allowed to age for approximately 24 hours.

All sensors are evaluated in an unsaturated mode since the time scale for reversibility may become an issue in a long term saturated mode and these longer term exposures are not necessary. NH₃ or NO were pulsed onto these interfaces with a 300 s half-cycle followed by a 300 s half-cycle nitrogen cleaning. The numbers denote ppm exposure to NH₃. The system was purged with UHP nitrogen for 1800 s before operation. Although we operate the sensors in an unsaturated mode, the sensor response and recovery times for “sticky gases such as ammonia are distinctly different and full time recovery from the gas exposure takes longer than 300 s, —the exposure time duration in the present configuration. However, the onset of the sensor response remains clearly visible. This behavior suggests that the response for NH₃ on PS is that of a gas whose interaction may be dominated by physisorption but which also displays weak chemisorption. Purging the sensor surface with UHP N₂ for longer durations improves the gradual shift to the initial base line. The return to baseline can also be further improved by more tightly constraining the gas flow path to the sensor surface. Nitridation with ammonia is a process that requires treatment for several hours at elevated temperatures^{2,3}. This process should be even more difficult with NO.

For all of the cases considered in this study, the analyte gas, either NH₃ or NO, that is sensed is brought to the hybrid surface after entrainment at room temperature in UHP nitrogen (Matheson 99.999+%). The system is purged with UHP nitrogen for a minimum of 30 minutes before use. The typical resistances for the base PS structures range between 300 and 10,000 ohms at room temperature. The gas flow for the analyte and the entraining UHP nitrogen are controlled by MKS type 1179 A mass flow controllers. The mass flow controllers used to control the analyte gas and the entraining nitrogen flow, respond to the gas in less than 2 seconds. The diffusion time of the analyte gas to the sensors, which provides the longest system time constant, varies from four to five seconds for the lowest analyte concentrations to of order 1 to 2 seconds for concentrations greater than 2ppm. These are the delay times for the observation of a signal due to the analyte in the supply line. The sensing interfaces respond to the analyte gas on a time scale much less than two seconds. The change in resistance is measured in one-second intervals using a DC current. This voltage bias used in these experiments is 3 volts to obtain an optimum signal to noise ratio. A NI DAQPad-6015 is used for gathering data and supplying the DC current. Labview software is used to control the experiment and record the results. MATLAB is used in the analysis of the data.

### REFERENCES

[1] R. Saferstein, Criminalistics, An Introduction to Forensic Science, 9^{th} edition, Pearson, Prentice Hall, Upper Saddle River, N.J. 2007.
[2] R. Asahi, T. Morikawa, T. Ohwaki, K. Aoki, Y. Taga, Science, 2001, 293, 269.
[3] F. Dogan, K. D. Hammond, G. A. Tomsett, H. Huo, W. C. Conner, S. M. Auerbach, and C. P. Grey, J. Am. Chem. Soc, 2009, 131, 11062-11079.
[4] R. Astala, S. M. Auerbach, J. Am. Chem. Soc. 2004, 126, 1843-1848.
[5] D. Lesthaeghe, V. Van Speybroek, M. J. Waroquier, J. Am. Chem. Soc. 2004 126, 9162-9163.
[6] D. Lesthaeghe, V. Van Speybroek, G. B. Marin, M. J. Waroquier, Phys. Chem. B. 2005, 109, 7952-7960 (2005).
[7] J. L. Gole, J. Stout, C. Burda, Y. Lou, X. Chen, J. Phys. Chem. B 2004, 108, 1230-1240.
[8] X. Chen, Y. Lou, A. C. S. Samia, C. Burda, J. L. Gole, Adv. Funct. Mater., 2005, 15, 41-49.
[9] J. L. Gole, S. M. Prokes, X. Qiu, C. Burda, J. Wang, O. J. Glemboki, Nanoscale, 2010, 2, 1134-1140
[10] J. L. Gole, S. E. Lewis, J. R. De Boer, P. J. Hesketh, Sensors and Actuators B, 2005, 110, 54-65.
[11] J. L. Gole, S. E Lewis, Nanosilicon, Kumar, S. Ed., Elsevier, London, 2007, pp. 147-192.
[12] J. L. Gole, S. Ozdemir, ChemPhysChem, 2010, 11, 2573-2581
[13] J. L. Gole, Photoluminescence Induced Metallization for Low Resistance Contacts, iTechnology Advances, Invited, MRS Bulletin 2003, 28, 263.
[14] P. A. Kottke, A. G. Federov, J. L. Gole, Multiscale Mass Transport in Porous Silicon Gas Sensors, Modern Aspects of Electrochemistry, Vol. 43, Schlesinger M., Ed., Springer, New York, 2009, 139-168.
[15] J. L. Gole, E. C. Goode, W. Laminack, ChemPhysChem, 2012, 13, 549-561.
[16] W. Laminack, N. Pouse, J. L. Gole, ECS J. Solid State Sci. Tech. 2012, 1, Q25-Q34. doi:10.1149/2.002202jss
[17] S. Ozdemir J. L. Gole, Current Opinions in Solid State and Materials Science, 11, 92-100 (2007)
[18] J. L. Gole, S. Ozdemir, T. S. Osburn, ECS Transactions, 2010 33 (8), 239-244.
[19] Gole, J. L.; Laminack, W. I. General Approach to Design and Modeling of Nanostructure Modified Semiconductor and Nanowire Interfaces for Sensor and Microreactor Applications, Chemical Sensors: Simulation and Modeling, Volume 3-Solid State Sensors, pp 87-136, Ed. G. Korotcenkov, Momentum Press, New York, USA.
[20] R. G. Pearson, Coord. Chem. Rev. 1990, 100, 403-25; R. G. Pearson, Chemical Hardness, John Wiley VCH, Weinheim, 1997.
[21] W. Laminack, J. L. Gole Advanced Functional Materials, DOI: 10.1002/adfm.201301250.
[22] a) R. G. Pearson, J. Am. Chem. Soc. 1963, 85, 3533-3539. B) R. G. Pearson, Chemical Hardness, John Wiley VCH, Weinheim, 1997. C) R. G. Pearson, J. Chem. Sci. 2005, 117(5), 369-377. R. G. Pearson, Inorg. Chem. 1988, 27, 734-740.
[23] Parr, R. G.; Pearson, R. G. Absolute Hardness: Companion Parameter to Absolute Electronegativity, J. Am. Chem. Soc. 1983, 105, 7512-7516.
[24] R. G. Pan, W. Yang, Density Functional Theory of Atoms and Molecules, Oxford University Press, New York, 1989.
[25] P. Geerlings, F. De Proft, W. Langenaeker, Chem. Rev. 2003, 109, 1793-1874.
[26] E. L. Lee, I. E. Wachs, J. Catal., 258 (2008), 103-110.
[27] M. Badlani I. E Wachs, Catalysis Letters 2001 Vol. 75, No. 3-4.
[28] D. A. Dixon, M. Gutowski, J. Phys. Chem. A, 2005, 109, 5129-5135
[29] E. P. Hunter, S. G. Lias, J. Phys. Chem. Ref Data, 1998, 27, 3, 413-656; B) S. G. Lias, J. E. Bartmess, J. F. Liebman, J. L Holmes, R. D. Levin, W. G. Mallard, J. Phys. Chem. Ref Data, Suppl. 1, 1988, 17, 1-861.
[30] N. C. Saha, H. C. Tompkins, J. Appl. Phys. 1992, 72,3072.
[31] NIST/EPA Gas-Phase Infrared Database.
[32] J. A. Rodriguez, T. Jirsak, J. Dvorak, S. Sambasioan, D. Fischer, J. Phys. Chem., 2000, 104B, 319.
[33] T. Jirsak, J. Dvorak, J. A. Rodgiguez, Surf Sci., 1999, 436, L683.
[34] L. A. DeLouise, N. Winograd, Surf Sci., 1985, 159, 199.
[35] C. D. Wagner, W. M. Riggs, L. E. Davis, J. R. Moulder, G. E. Muilenberg, Handbook of Xray Photoelectron Spectroscopy, Perkin-Elmer: Edev-Praire, Minnesota, 1978, 40.
[36] E. Gyorgy, A. Perez del Pino, P. Serra, J. L. Morenza, Surface and Coatings Technology, 2003, 173, 265.
[37] J. Campbell, J. A. Como, N. Larsen, J. L. Gole, J. of Electrochem. Soc., 2008 155, D128-D132.
[38] S. Ozdemir, J. L. Gole, Sensors and Actuators B, 2010 151, 274-280.
[39] C. Levy-Clement, A. Lagoubi, M. Tomkiewicz, J. Electrochem. Soc. 1994 141, 958-967.
[40] H. Foell, M. Christophersen, J. Carstenson, G. Hasse, Materials Science and Engineering R280, 149, (2002).
[41] S. Ozdemir, T. Osburn, J. L. Gole, J. Electrochem. Soc., 2011 158 (7), J201-J207.

### Example 2

### Brief Introduction

Nanostructure decorated n-type semiconductor interfaces are studied in order to develop chemical sensing with nanostructured materials. We couple the tenents of acid/base chemistry with the majority charge carriers of an extrinsic semiconductor. Nanostructured islands are deposited in a process that does not require self-assembly in order to direct a dominant electron transduction process that forms the basis for reversible chemical sensing in the absence of chemical bond formation. Gaseous analyte interactions on a metal oxide decorated n-type porous silicon interface show a dynamic electron transduction to and from the interface depending upon the relative strength of the gas and metal oxides. The dynamic interaction of NO with TiO₂, SnO₂, NiO, CuₓO, and AuₓO (x>>1), in order of decreasing acidity, demonstrates this effect. Interactions with the metal oxide decorated interface can be modified and controlled by the in-situ nitridation of the oxide nanoparticles, enhancing the basicity of the decorated interface. This process changes the interaction of the interface with the analyte. The observed change to the more basic oxynitrides does not represent a simple increase in surface basicity but appears to involve a change in molecular electronic structure which is well explained using the recently developed IHSAB model. The optical pumping of a TiO₂ and TiO₂₋ₓNₓ decorated interface demonstrates a significant enhancement in the ability to sense NH₃ and NO₂. Comparisons to traditional metal oxide sensors are also discussed.

### Introduction:

The combination of tailored active interfaces, the ability to confine processes at the nanoscale, and the ability to manipulate nanostructured materials and their interaction at these select interfaces, offers the opportunity to develop economically viable, energy efficient, and sensitive devices for the direct sensing of a variety of chemical species. Processes that are driven by nanostructure-focused Brönsted and Lewis acid-base chemistry can provide rapidly responsive and sensitive (ppb) sensor platforms.¹⁻⁴ Within this framework, the creation of highly active, nanopore coated microporous extrinsic semiconductor interfaces, their ability to provide readily accessible significant light harvesting surface areas,⁵ and their ability to be transformed with selective nanostructure sites, enables sensing based on efficient electron transduction. Decorated microporous arrays enable enhanced analyte diffusion to active sites,⁶ whereas the nanopores provide a “phase matching” region with which the modifying nanostructured materials of interest can be made to interact in a controlled manner to promote a range of interface sensitivities.

The selection of the appropriate nanostructured materials relies primarily on the IHSAB model,³ based on concepts from hard/soft acid-base theory to develop model nanostructures which, within themselves or deposited on high surface area interfaces, (1) provide a range of selectable sensitivities^{3,7} to a variety of analytes and (2) provide a sensitive and dynamic⁸ mechanism for electron transduction. The inverse hard and soft acid/base (IHSAB) model links chemical selectivity and the mechanism of sensor response, for nanostructure modified and directing acidic or basic sites on microporous extrinsic semiconductor channels, through fractional deposition of the nanostructures. These nanostructures do not form a surface coating but rather act as independent nanostructured sites capable of strongly directed interaction with a given analyte and subsequent rapid electron transduction. In this configuration, the basic tenants of acid/base chemistry (the ability of Lewis bases to donate electrons and Lewis acids to accept electrons) and semiconductor physics can be coupled to provide a road map for the implementation of readily constructed, energy and cost efficient, rapidly responding, devices which can be sensitive to the ppb level. The selection of the nanostructures that are deposited and the variable surface sensitivities that are produced, as they form in situ metal/metal oxide deposits, can now be largely predicted. Further, the deposited sites can be modified in-situ to form the corresponding oxynitrides, with a greatly increased basicity. Using a defined procedure, based on established molecular and semiconductor properties, the IHSAB model dictates the coupling of analyte/interface acid-base interactions with the properties of the majority charge carriers in an extrinsic semiconductor. When such properties are not already available, it is possible to use advanced computational chemistry approaches for their prediction, to improve our understanding of the dynamics of electron transduction across the interface, and to analyze the changes in molecular electronic structure that this process induces. In combination, this provides a focused chemistry which tailors electron flow at the interface, differentiates electron transduction vs. chemisorption, and can enhance light harvesting efficiency. This approach is now developed to the extent that the dynamics of analyte-decorating nanostructure-interface interactions and the nature of competitive electron dynamics can be evaluated.⁸

We emphasize that the nanostructures are deposited to form islands on the micropores of the extrinsic semiconductor and suggest that the confined nature of these nanostructured islands is fundamental to their initial strong interaction and efficient electron transduction. The fractional deposition does not require time-consuming self-assembly within the pores of the interface, and is far simpler to implement than traditional thin film or alternative “coating” techniques. This approach creates a distinct sensor platform where the nanostructures control and focus a variable and efficient analyte interaction and the transfer of electrons to or from the extrinsic semiconductor interface. The in-situ transformation of the deposited nanostructured metal oxides to their corresponding oxynitrides^{8,9}, as it introduces basicity, also facilitates the change of sensor response through optical pumping⁹⁻¹¹. The extrinsic semiconductor is, however, independently variable with a distinctly different band structure and electron dynamics associated with n or p-type doping. Although these two components are separable, they can be combined to provide an enhanced versatility versus a single or mixed metal oxide surface coating. In concert with the IHSAB principle, this approach leads to an optimized and simpler interface. Treatment of the semiconductors with nanostructured photocatalysts can be used to facilitate the use of the system for solar pumped sensing.^{7,11} The control of the interaction of targeted analytes with a specific material and the degree to which this dictates the tailoring of interfaces through the understanding of their physics and chemistry offers a uniquely defined approach to enable the selection of device materials and a general framework for the design of advanced sensor platforms.

### The Sensor Interface:

### Micro/Nano-Porous Semiconductor Surface:

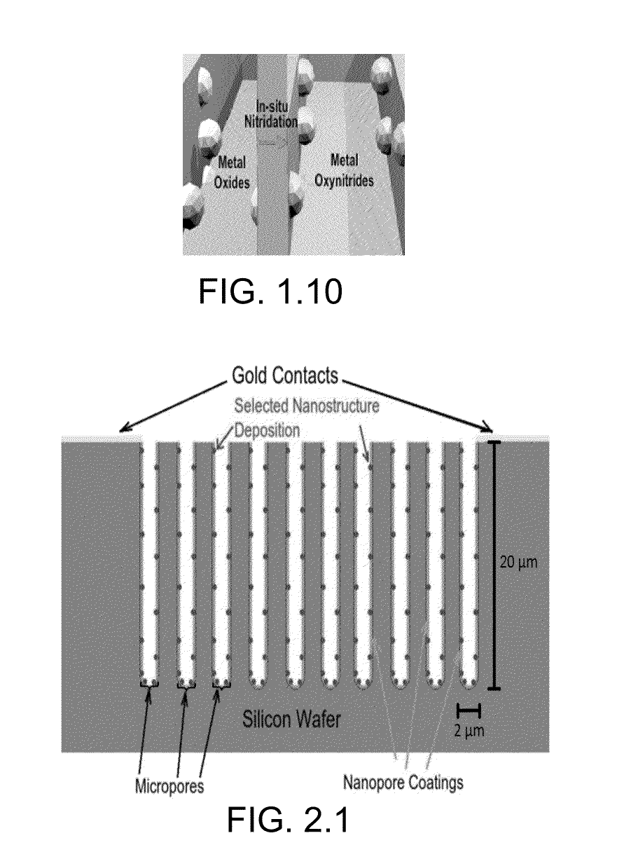

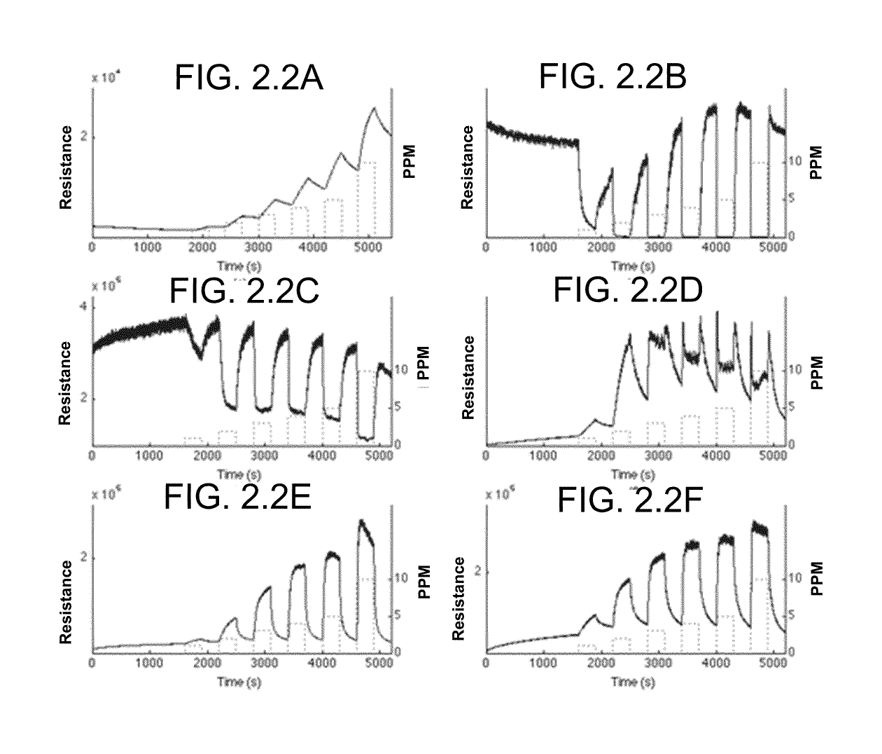

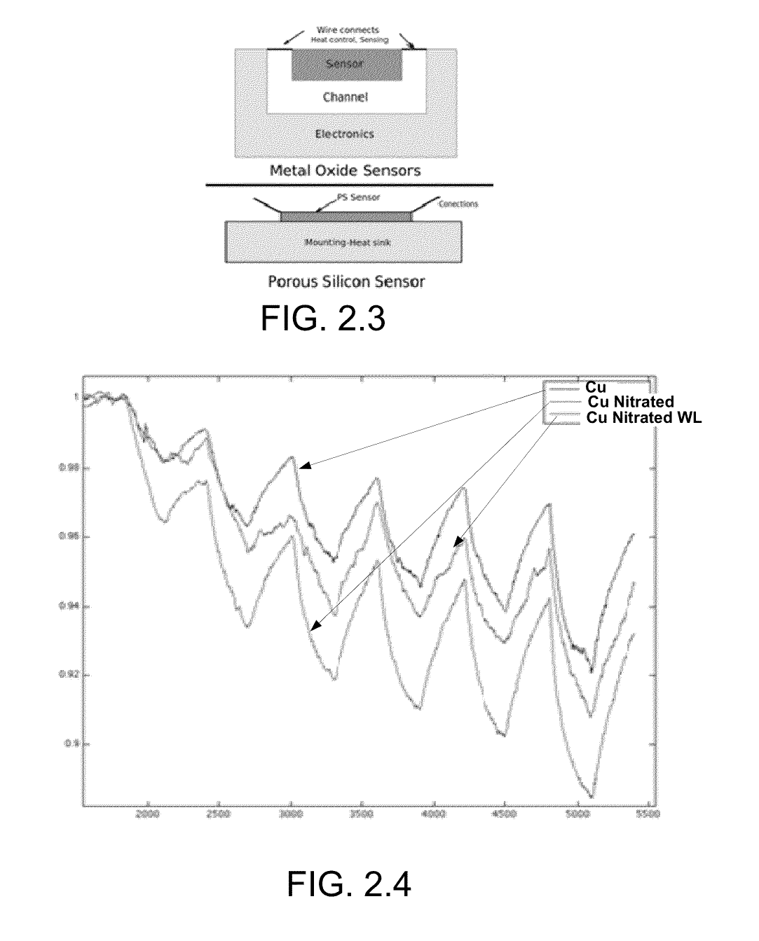

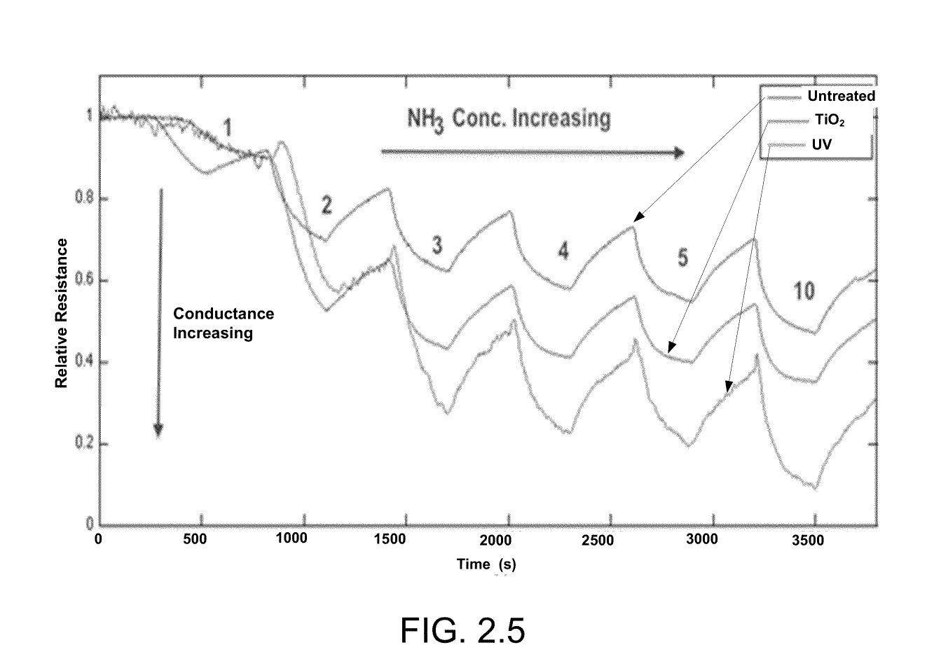

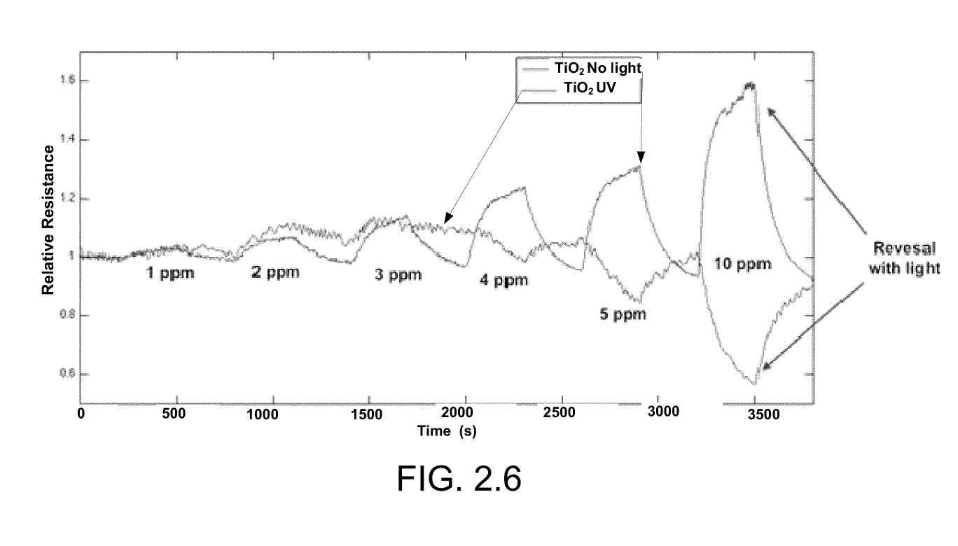

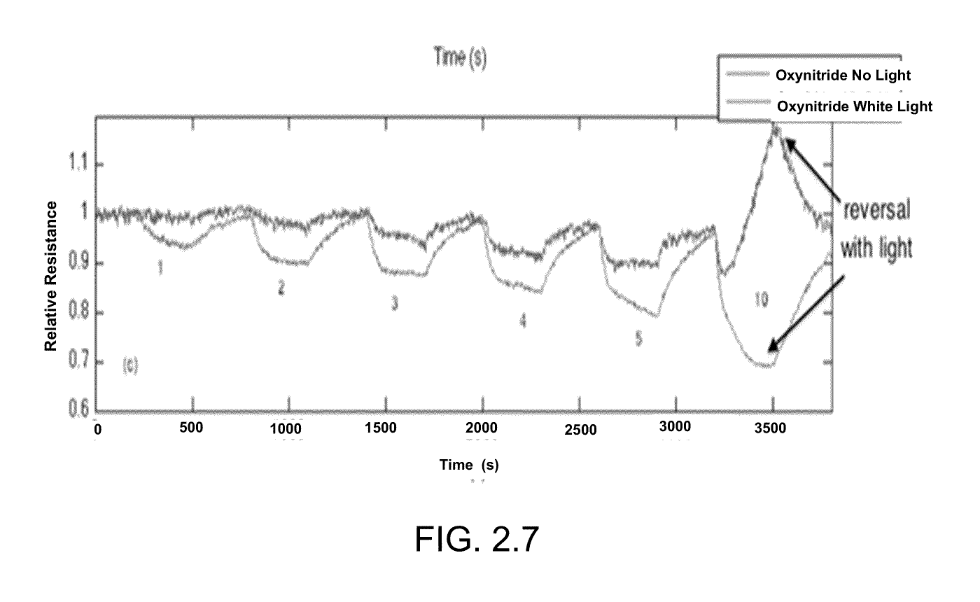

The semiconductor interfaces of interest are illustrated by the porous silicon (PS) nano/microporous interface depicted in FIG. 2.1. This structure is produced by a hybrid etch procedure used to create the desired interfacial support structure. The silicon structure or layers and the porous silicon (PS) structures, devices, and methods, considered in the following discussion can be replaced with any alternate extrinsic semiconductor (e.g., GaP, InP, CdTe)¹² onto which a porous microstructure can be generated. Further, the configuration in FIG. 2.1 can be adapted to a pass-through microporous membrane.¹³ This greatly broadens the range of nanostructure-interface combinations which can be potentially exploited as the alternate extrinsic semiconductors can be a p-type substrate, a p⁺-type substrate, or an n-type substrate. The nanopore covered microporous structure of the interface has been created specifically to facilitate efficient gaseous diffusion (Fickian)⁶ to the highly active nanostructure modified nanoporous coating. The nanoporous coating acts to provide a phase matching for the subsequent deposition of selected nanostructured islands (FIG. 2.1-red). These surface-attached nanoparticles possess unique size dependent and electronic structure properties that form a basis for changing the sensitivity for exposure to specific gases. This exposure alters the conductivity of the porous silicon or an alternate extrinsic semiconductor (measured by microprobe circuitry) attached to the gold contacts shown in FIG. 2.1. While the room temperature operation of this interface serves many applications, the appropriate installation of heat sinks allows operation at several hundred degrees C. A developed interface thus operates under conditions which are not amenable to typical metal oxide systems. Especially when coated with nanoparticle photo-catalysts, the interface could also operate as a nanostructure-modified microreactor for efficient chemical transformation. When operated in the electron transduction mode, the transfer of electrons to an n-type PS interface, as would occur with a basic analyte, enhances the majority carriers which are electrons, decreases the conductmetric resistance and increases conductance. The removal of electrons, as would occur with an acidic analyte, decreases the majority charge carriers and the conductance and increases resistance. The opposite behavior will be observed for a p-type semiconductor interface.

### Inverse Hard and Soft Acid/Base Concept:

We have applied the inverse hard and soft acid and base (IHSAB) concept,^{3,7,8} using metal oxide and oxynitride nanostructure modified interfaces. This concept complements the tenants of HSAB interactions.¹⁴ It includes the coupling of analyte/interface acid-base chemistry with select interfaces, leading to a balance and separation of surface electron transduction and chemisorption, and enables the ability of active nanostructure-based sites to utilize these differences. Based on the reversible interaction of hard acids and bases with soft bases and acids the IHSAB principle enables the selection of interacting materials that do not form strong covalent or ionic chemical bonds and complements the HSAB model¹⁴ for significant bond formation based on strong ionic (hard acid/base) or covalent (soft acid/base) interactions. As an extrapolation of the HSAB concept developed by Pearson¹⁴ and later correlated within the context of density functional theory (DFT) by Pearson,¹⁵ Parr, and others,^{16,17,18} the IHSAB model is somewhat broader-based and predicts reversible sensor-analyte interactions. The details of this model are given elsewhere^{3,7}. The fractional deposition of TiO₂, SnO₂, NiO, CuₓO, and AuₓO (x>>1) nanostructured islands (FIG. 2.1) modifies the response of an intrinsic porous silicon interface to increase sensitivity. Some typical responses, represented as the ratio of the observed signal compared to that of porous silicon are summarized in Tables 2 and 3. These ratios, while they are for a given interfacial structure^{3,7} are maintained as one improves the pore structure of the interface to produce sensors which operate at the ppb level.

TABLE 2
Relative increase in response (increase in resistance) of SnO₂, NiO, CuₓO,
and gold clustered oxide, AuₓO treated “p-type” PS interfaces relative
to the untreated interface. The table constitutes a response matrix to
the gases PH₃, NO, NH₃, and SO₂.^{3,7}

| | SnO₂ | NiO | CuₓO | AuₓO |
|---|---|---|---|---|
| PH₃ | 2 | 2.5 | 4 | 5 |
| NO | 7-10 | 3.5 | 1 | 1.5-2 |
| NH₃ | 1.5 | 1.5-2 | 2-2.5 | ~3 |
| SO₂ | 4 | (2) | 1+ | 2 |

TABLE 3
Relative increase or decrease in resistance (decrease or increase in
conductance) of TiO₂, SnOₓ, NiO, CuₓO, and gold clustered oxide,
AuₓO treated “n-type” PS interfaces. The table constitutes a
response matrix for the gases NO, NO₂, and NH₃.

| | TiO₂ | SnO₂ | NiO | CuₓO | AuₓO |
|---|---|---|---|---|---|
| NO | −12* | −2* | 4 | 1.2 | 1.5-2 |
| NO₂ | 0.75 | 0.5** | (0.9-1) | 1 | 1.5-2** |
| NH₃* | (3.5-4) | 2.5 | 1.5 | 2 | 3 |

*indicates decrease in resistance with analyte exposure
**indicates initial response.⁷

The relative responses given in Tables 2 and 3 can be correlated to allow the construction of the a “Materials Positioning Diagram” for the acids and bases within the IHSAB and HSAB concepts as summarized in FIG. 1.1. Recently, we have obtained additional data for PH₃ on p, n, and p⁺ decorated porous silicon (PS)¹⁹. For p-type PS, a TiO₂ decorated surface is five times more responsive than the untreated PS interface. For n-type PS, TiO₂ and AuₓO decorated surfaces are respectively 2-2,5 and 3-5 times more responsive. For p⁺-type PS, TiO₂, SnO₂, CuₓO, and AuₓO decorated surfaces are respectively 4,2, 2-2.5, and 7 times more responsive. The analyte response data forms the basis for the development of the materials positioning diagram^{3,7} based largely on the interaction of the acidic metal oxides ranging from TiO₂ to AuₓO (x>>>1) and the bases NH₃ to CO. The relative separation of the oxides and the bases within the range from hard to soft acids and bases dictates the observed responses of the interface. NH₃ displays a maximum reversible response for an AuₓO deposited surface whereas CO displays a maximum response for TiO₂ and SnO₂. Thus, in contrast to chemical bond formation, the greatest reversible response corresponds to the largest molecular orbital mismatch^{3,7}. The combination of responses for the analytes considered form the basis for selectivity based the combinatorial arrangement of arrays of decorated n, p, and p+-type PS interfaces, for which the interfacial structure of FIG. 2.1 can be generated.

The nitridation of the metal oxides can be used to modify the nanostructure island site basicity through in-situ transformation to the corresponding oxynitrides. The degree of nitridation can be used to introduce a progressively increasing basicity. The transformation is accomplished in a manner analogous to that applied to the facile conversion of nanostructured TiO₂ to TiO₂₋ₓNₓ^{9,10}. The in-situ formation of the oxynitrides shifts the positioning of the oxides toward the soft acid side of FIG. 1.1 as it promotes the formation of more basic sites. This promotes a significant change in sensor response.

### Results and Discussion:

Nitridation Concept and Enhanced Basicity Associated with the Formation of Oxynitride Surfaces.

The enhanced basicity inherent to the oxynitride systems that we are developing can be demonstrated in multiple ways. Consistent results are obtained from the measurement of the in-situ change in response resulting from nitridation as predicted by the IHSAB concept and its correlation with an enhanced basic character, gauged also by the softening of acidity. Further, by examining the surface chemistry of nitridated nanostructures and applying the decomposition reaction of methanol, it is possible to distinguish acid and base sites and therefore the transformation from acidic to basic sites. These studies also define a broadened interaction matrix as it extends from physisorption (sensing) applications to chemisorption and microreactor design.

Recently, we have produced visible light absorbing TiO₂ₓNₓ photocatalyst nanoparticles in seconds at room temperature using alkyl ammonium compounds^{9,10,20,21}, leading to the direct nitridation of highly porous TiO₂ nanocolloids. The observed effect of in-situ nitridation as it modifies the response of a semiconductor interface within the IHSAB format^{3,6,7,22-25} can be demonstrated as nanostructured TiO₂ represents a strong (hard) acid. Its oxynitride, TiO₂₋ₓNₓ, once formed, through in-situ treatment of a TiO₂ deposited surface, has gained considerable basic character. The data in FIGS. 1.3A and B compare the response of an untreated n-type PS interface, upon exposure to 2-10 and 20 ppm of NH₃, and that for the interface treated with a deposition of acidic” TiO₂ nanostructures, and this same interface where the deposited nanostructures are converted from TiO₂ to the more basic TiO₂₋ₓNₓ. TiO₂, as a strong acid, enhances the capture of electrons, transferring these electrons to increase conductance (decrease resistance) relative to the undecorated interface. The more basic oxynitride does not facilitate electron transduction as efficiently and the sensor response corresponds to a conductance decrease relative to the untreated interface. Note also that the in-situ nitridation of TiO₂ shifts the nature of this metal oxide nanostructure toward the soft acid side of FIG. 1.1, closer to ammonia. The IHSAB principle dictates^{3,6,7,22-25} that the orbital matchup with NH₃ is enhanced and therefore the reversible response of the TiO₂₋ₓNₓ interface should decrease relative to TiO₂ as it does. Similar decreases in the observed sensor response are observed as nitridated SnO₂ interacts with NH₃ and NO with which its molecular orbital makeup is now more closely aligned. The nitridation of NiO also leads to a decrease in response for NO, however, the reversible response for interaction with NH₃ increases. FIG. 2.4 presents comparable data as 1-10 ppm of ammonia interacts with a copper oxide treated n-type PS interface converting this interface in-situ to a copper oxynitride interface. Again, the nitridation of CuₓO forms more basic sites and shifts the response of the modified nanostructures further to the soft acid side of FIG. 1.1. It is tempting to suggest that the formation of the oxynitride should simply increase the basicity of the nanostructure surface and thus should decrease the response to NH₃. However, this does not occur. The nitridated copper oxide is shifted further to the soft acid side of ammonia in FIG. 1.1, dictating a greater mismatch of molecular orbitals. The IHSAB principle suggests, counter to intuition, that the response of the in-situ treated nitridated copper oxide interface should increase relative to that of CuₓO, precisely as is observed. In FIG. 1.1, NO is positioned directly under the copper oxides. Nitridation shifts the copper oxides to the soft acid side and away from NO, leading to an increase in molecular orbital mismatch and the reversible response of the oxynitride to NO. These results strongly suggest that the IHSAB principle can be used as an important distinguishing principle of sensor response.

### Microcatalysis of Metal Oxide and Oxynitride Surfaces:

We have examined several of the metal oxide and oxynitride samples for the qualitative aspects of their surface chemistry using the methanol decomposition reaction. This reaction is not a replacement for titrations with model acid and base compounds, however, the MeOH probe reaction has been effectively used by Wachs^{26,27} to characterize the surface of bifunctional, mixed metal oxides. They have demonstrated the utility of evaluating redox, acid, and base sites on surface opened reaction manifolds leading to the products: formaldehyde, dimethyl ether, and CO/CO₂, respectively.

2CH₃OH→CH₃OCH₃+H₂O: acid sites

CH₃OH→HCHO+H₂: redox sites

CH₃OH→CO+2H₂: base sites

Using these probe reactions, we have discerned that the nitridation process offers the opportunity to convert the metal oxide acid sites to more basic metal oxynitride surface sites.

### Optical Pumping of a Nanostructure Modified Porous Silicon Interface:

It is possible to enhance the sensitivity of an n-type extrinsic semiconductor PS interface to which TiO₂ and TiO₂₋ₓNₓ photocatalytic nanostructures have been deposited. PS sensor interfaces can be treated to form TiO₂ nanostructure island sites that greatly enhance the surface acidity and sensitivity to NH₃, FIG. 2.5 demonstrates that the sensitivity to NH₃ greatly increases as UV light impingent on the sensor increases the acidic character of TiO₂. NO₂, as a moderate acid is found to extract electrons from a PS interface^{7,25} and treatment with moderate concentrations of TiO₂ enhances the response to NO₂. However, FIG. 2.6 demonstrates that UV light now reverses this process as the enhanced acidity of an optically pumped TiO₂ treated interface begins to extract electrons from the moderately acidic NO₂. In-situ nitridation of the TiO₂ to form the oxynitride, TiO₂₋ₓNₓ, enhances the visible light response, basicity, and sensitivity of a decorated PS interface. At low fractional TiO₂ depositions, NO₂ dominates TiO₂₋ₓNₓ and white light excitation increases the sensor response in the form of an increased resistance. In contrast, at higher fractional depositions, FIG. 2.7 demonstrates that white light now increases the sensor response in the form of an increased conductance as the TiO₂₋ₓNₓ decorated interface is found to extract electrons. With light intensities less than a few lumens/cm²-sterad-nm, responses are enhanced by up to 150% through interaction with visible (and UV) radiation. These light intensities should be compared to the sun's radiation level, ˜500 lumens/cm²-sterad-nm suggesting an important extension of the IHSAB principle and the possibility of solar pumped sensing. The results we obtain with optical pumping not only follow the tenants of the IHSAB principle but they suggest an intriguing electron dynamics which pervades through these systems.

### Dynamics of Nanostructured Metal Oxide Analyte Interaction:

We have previously considered the dynamic interaction and competition between NO₂ and a TiO₂ nanostructure modified n-type PS interface. The addition of NO₂, which extracts electrons, leads to a resistance increase (conductance decrease). However, a fractionally deposited strong acid such as TiO₂ can compete effectively with the moderately strong acid, NO₂, for the available electrons in this system.^{3,8} As NO₂ is introduced to the decorated PS interface and attempts to extract electrons, the sensor resistance rises rapidly to a point when the electron depletion reaches a limiting value as nanostructured TiO₂ islands coupled to the PS interface prevent further electron withdrawal and reverse the flow of electrons so as to increase the donor and conduction level electron concentrations. This can lead to a sharp decrease in the resistance. The process of interaction is a dynamic one as TiO₂ and NO₂ vie for the available electrons as the NO₂ is introduced and removed from the system. Further the process of electron withdrawal is strongly influenced by the relative concentration of the TiO₂ island sites. A similar dynamic playoff is observed with the amphoteric NO radical which can be either a weak acid or a weak base.^{7,25}

FIG. 2.2A which shows a positive resistance change with concentration indicates that, when brought in contact with an n-type PS interface, NO acts like a weak acid. The boxes in the figure indicate the concentration range from 1 to 5 ppm. Nanostructure treated PS interfaces deposited with the acidic metal oxides in decreasing strength TiO₂>SnOₓ>NiO>CuₓO>AuₓO (x>>1) demonstrate not only a starkly different response but also clear trends which can be associated with the relative acid strengths of the metal oxides. FIGS. 2.2B and 2.2C indicate the responses for the PS interfaces decorated with TiO₂ and SnOₓ. The strong acid character of TiO₂ and to a lesser degree SnO₂ has overcome the ability of NO to extract electrons. Instead, electrons are extracted from NO and transferred to the PS interface to greatly increase conductance. Although the observed responses are virtually linear to 5 ppm for the untreated PS interface, they are clearly quenched for the TiO₂ and SnO₂ surfaces at NO concentrations in excess of 4 ppm. The observed response at 10 ppm diminishes for TiO₂ which suggests that the ability of this interface to extract electrons from NO has reached a limiting value. This is less apparent for SnO₂ but the ability of the interface to extract electrons is still significantly diminished. The magnitude of the signals observed for the TiO₂ and SnO₂ interfaces, while opposite in sense, exceed those for the untreated PS interface by factors of 12 and 2 respectively.^{7,25}

FIG. 2.2D corresponds to the response of a PS interface treated with the intermediate acid NiO. The response displays an intriguing intermediate behavior as NO and the decorated PS interface now complete effectively for electrons. At the lowest NO concentrations the response of the n-type PS interface is enhanced as the ratio of the responses (2ppm/1 ppm) increases significantly relative to the n-type PS as the observed response rises to a maximum throughout the cycle of NO exposure. At an NO concentration in excess of 3 ppm, the dynamic response at first increases rapidly to a sharp maximum, subsequently decreases, oscillates, and then increases as the NO concentration decreases. The dynamic behavior is even more pronounced at 4, 5, and 10 ppm. As the NO concentration increases, the transfer of electrons to NO increases to a maximum, indicated by the onset of the spike-like features which diminish in width with increasing NO concentration. We suggest that this results as the transfer of electrons to NO reaches a limit when the n-type PS interface is sufficiently depleted so that it acts as a stronger acid than the NO radical. At this point electrons are extracted from NO (acting as a base), accompanied by a decrease in the measured dynamic resistance (increase in conductance) as the semiconductor donor levels are repopulated. The oscillatory behavior which is especially apparent at concentrations of 4, 5, and 10 ppm is suggested to result from a continual if not less pronounced change of the competing interface and NO. This will be the subject of further study. The time dependent competition between NO and the NiO decorated interface means that the sensors return slowly to a baseline response.

FIGS. 2.2E and 2.2F demonstrate a clear trend in the responses of the weaker acid nanostructure deposited PS interfaces, based on CuₓO and AuₓO nanostructure depositions and an increase the response of the PS interface by factors of 1.2 to 1.5 respectively.^{7,25} The increase in response is, as expected, greater for the weaker acid AuₓO. The CuₓO and AuₓO decorated interfaces thus act to enhance the electron withdrawing power of the NO radical, which suggests that they represent weaker acids on the n-type PS interface. Consistent with trends in the acid strength of the nanostructure deposits, the CuₓO response increases to a maximum with NO exposure for concentrations 1-4 ppm, however, this increase is slowed at 4 ppm. At 5 ppm the response peaks at an intermediate time of the NO exposure and at 10 ppm, the signal peaks shortly after the NO exposure and, counter to the behavior at lower pressures, decreases. This overall behavior is consistent with the weaker acid nature of CuₓO vs. NiO. At 4 ppm, the CuₓO decorated interface begins to compete effectively with NO for the available electrons and at 10 ppm the interface is able to overcome the extraction of electrons by NO as the response shows a decrease with time of NO exposure. Note also the almost complete return to baseline.

The trends that we describe for CuₓO extend to the weaker acid AuₓO decorated interface. Here, FIG. 2.2F demonstrates that AuₓO response increases to a maximum with exposure to NO concentrations 1-4 ppm. At 5 ppm the response peaks at an intermediate response to the NO exposure. However, at 10 ppm the response decreases with NO exposure but at a much slower rate than does the response for CuₓO. The extraction of electrons by NO is overcome but to a much lesser extent. Note also the return to baseline.

The variations in response observed for the nanostructure treated PS interfaces, while reflecting the donor level population, demonstrate the important role played by the deposited nanostructures and the nature of acid/base interaction they direct. The observed trends also correlate well with relative responses observed as NO interacts with p-type PS. Within this framework the behavior of the NiO decorated interface is indeed intriguing. At the lowest concentrations the NiO nanostructure deposited interface mimics the dynamic behavior of the weak acids CuₓO and AuₓO and at higher concentrations it is transmuted to a surface similar to that treated with the stronger nanostructured acids TiO₂ and SnOₓ. As a function of concentration, NO acts as both an acid and a base.

### Comparison to Traditional Metal Oxide Sensor Systems:

In contrast to traditional metal oxide systems, the present systems create a dual interface where the nanostructured islands and the extrinsic semiconductor act separately but are coupled. Our room temperature operative design, adds considerable flexibility not possible in a singly or multiply “coated” metal oxide interface. There is yet another important distinction. Metal oxide sensors (FIG. 2.3), (when compared also to electrochemical sensors) are slightly less costly to produce, however, concerns may include poor sensitivity, high power requirements, and most importantly, the need to operate at elevated temperatures. The latter requirement is a drawback for several reasons. First, a power consuming heating element must be provided with the sensor housing to precisely control the temperature of the sensor element. This is, in large part, intimately tied to the correct identification of the gas of interest. Distinguishing one gas from another thus requires that the heating element and sensor be well separated (channel) from the remaining electronics. This means that this configuration can be greatly affected by an impinging combustion or flue gas, rendering difficult the correct identification of gaseous species in the flow. In contrast, the PS sensor configuration depicted in FIG. 2.1 consumes less power, is far simpler, and does not require the complexity of a system separated sensor/heater configuration. In a heat sink environment (FIG. 2.3), it is potentially capable of operation in a high temperature gas flow.

### Development of Materials Selection Tables:

It would seem appropriate to expand the selective deposition of nanostructured materials to create inexpensive microfabricated sensor platforms and develop “materials selection tables” built on the IHSAB model. The response data that we have outlined form the basis for the development of an initial materials positioning diagram (FIG. 1.1) predicted by the IHSAB concept. It remains to expand the metal oxide data base, including the in-situ transformation to the corresponding oxynitrides, to enhance the array of distinct responses which can be developed and extended to form “materials sensitivity matrices” for a given analyte. This will enhance the capability to sense analytes and their mixtures. This ready transformation is easily accomplished in a manner analogous to that applied to the facile conversion of TiO₂ to TiO₂₋ₓNₓ.^{9,10} Because the in-situ formation of the oxynitrides will shift the positioning of the oxides toward the soft acid side of FIG. 1.1, it will add a notable flexibility to the materials sensitivity table. Results suggest that the nitridation process does not simply increase the basic character of the nanostructure surfaces but that it modifies the molecular structure and interaction as the metal oxide deposited surface has gained considerable basic character. Initial results suggest that the nitridation process does not simply increase the basic character of the nanostructure surfaces but that it modifies the molecular structure and interaction consistent with the IHSAB principle. This means that the sensitivity of the weaker metal oxides is enhanced by nitridation. Further, this process can be applied to create several potential visible light absorbing photocatalysts similar to TiO₂₋ₓNₓ.^{9,10}

We wish to better understand the change in electronic character of the sensing system when it interacts with an analyte and the analyte injects or removes charge from the semiconductor interface to change the resistance. It is of interest to understand how the occupied bands in the semiconductor change when the analyte interacts with the surface. How does the change in the bands occur? How does this affect the band gap? The prediction of the electronic properties, especially the band gaps, is closely tied to the actual structure of the interface. Where does the analyte bind to the interface? What types of interactions dominate the analyte-interface bonding in, for example, the competition between physisorption (electron transduction) and chemisorption? The IHSAB concept appears to map a general approach to the development of sensor systems, however, it remains to obtain a more quantitative picture of these systems.

### Conclusion:

We have demonstrated the efficacy of fractional nanostructure depositions as a means of obtaining distinct sensor responses which show the potential for combination in an array based format. The behavior of these systems appears to be well represented by the newly developing IHSAB model. We have also considered the conversion of the metal oxides in-situ to their oxynitrides and the enhanced basicity that this introduces to a nanostructure decorated PS interface. These systems also display a time-dependent dynamics which must be incorporated into the IHSAB model. This will be the subject of future studies.

### Experimental:

Highly efficient nanostructure modified interfaces on either p- or n-type PS, as we generate the micro/nanoporous interface outlined in FIG. 2.1³. A hybrid etch procedure is used to generate nanopore covered micropores. Schematic diagrams of the complete working sensor platform have already been presented^{3,13,23}. The PS interface is generated by electrochemical anodization of 1-20 ohm-cm, n-type (phosphorous doped) (100) silicon wafers (Wafer World) or 7-13 ohm-cm, p-type (boron doped) (100) silicon wafers (Siltronix). The anodization of the n-type wafers^{28,29} is done under topside illumination using a Blak-Ray mercury lamp. The silicon wafer is etched in a 1:1 solution of HF and ethanol at a current between 8-15 mA/cm^{23,24,28,29}. The anodized n-type sample is placed in methanol for a short period and subsequently transferred to a dilute HF solution for a 30 minute period. This process creates a porous structure with pore diameters of order 0.5-0.7 m and pore depths varying from 50 to 75 μm. The mircopores provide a medium for Fickian diffusion to the surface nanoporous layer.

Before the anodizations, an insulation layer of SiC (≈1000 angstroms) is coated onto the c-Si substrate by PEVCD methods. Windows of size 2×5 mm are opened in this layer by Reactive Ion Etching (RIE). The SiC layer serves two purposes. SiC makes it possible to form the hybrid micro/nanoporous PS structure in the 2×5 mm windows during electrochemical anodization because of its resistance to HF. The SiC also aids the placement of gold contacts exclusively on the porous layer for resistance measurements and acts as an electrical insulator on the doped silicon. The PS hybrid arrays of nanopore covered micropores are tested at room temperature for their individual sensor response. The nature of this response is based on the application of the IHSAB acid/base principle. The selection of the nanostructures and the variable surface sensitivities that are produced as they form in-situ metal oxide deposits, introduces a distinct systematics of design, which can be predictably formatted. The approach is unique in that the nanostructures are deposited fractionally to the PS micropores and this fractional deposition DOES NOT require any time consuming self-assembly within the pores. This is not a coating technique or one that requires an exacting structural film arrangement but is, in fact, a much simpler process. The nanostructure deposition must be maintained at a sufficiently low level to avoid cross-talk between the nanostructures that, as it increases, leads to a noisy device and the eventual loss of functionality. In combination, these can be used as a basis to develop selectivity. Results obtained with nanostructured deposits generated from electroless gold, tin, nickel, and copper, as well as nanotitania are considered in this study.

With the exception of the gold depositions, all of the nanostructured metals deposited to the PS surface are readily oxidized to SnOₓ (x=2,4) and CuₓO (x=1,2) as demonstrated by XPS measurements²³. The initially introduced titania (anatase) may be crystalline, however, we cannot be certain of this crystallinity after deposition to the PS interface. The untreated PS hybrid structures are exposed to the electroless solutions for 10 to 30 seconds and are placed in DI H₂O and MeOH for consecutive 120 second periods. The oxidized electroless metal depositions when characterized before deposition correspond to amorphous structures displaying no diffraction patterns. Therefore, it is difficult to envision their crystallization during the short deposition and subsequent surface cleaning process. After deposition the decorated surfaces are cleaned for 120 s in DI and 120 s in methanol. Basic character is introduced to the nanostructured metal oxides by direct in-situ treatment with triethylamine (TEA). The metal oxide treated surface is exposed to the TEA for 10 seconds. The treated interface is subsequently washed in methanol to remove excess TEA and allowed to age for approximately 24 hours.

In all cases, the analyte gas being sensed is brought to the hybrid surface after entrainment at room temperature in UHP nitrogen (Matheson 99.999+%). The system is purged with UHP nitrogen for a minimum of 30 minutes before use. The typical resistances for the base PS structures range between 300 and 10,000 ohms at room temperature. The gas flow for the analyte and the entraining UHP nitrogen is controlled by MKS type 1179A mass flow controllers. The mass flow controllers used to control the analyte gas and the entraining nitrogen flow responsed in less than 2 seconds. The diffusion time of the analyte gas to the sensors, which provides the longest system time constant, varies from four to five seconds for the lowest anaylte concentrations to of order 1 to 2 seconds for concentrations greater than 2ppm. These are the delay times for the observation of a signal due to the analyte in the supply line. The sensors respond to the analyte gas on a time scale much less than two seconds. The change in resistance is measured in one-second intervals using a DC current. This voltage bias used in these experiments is 3 volts to obtain an optimum signal to noise ratio. A NI DAQPad-6015 is used for gathering data and supplying the DC current. Labview software is used to control the experiment and record the results. MATLAB is used in the analysis of the data.

### REFERENCES

1. Gole, J. L.; Lewis, S. E.; De Boer, J. R.; Hesketh, P. J. Sensors and Actuators B, 2005, 110, 54-65.
2. Gole, J. L.; Lewis, S. E. “Porous Silicon—Sensors and Future Applications,” in Nanosilicon, Kumar, S. Ed., Elsevier, London, 2007, pp. 147-192.***
3. Gole, J. L.; Ozdemir, S., ChemPhysChem, 2010, 11, 2573-2581.***
4. Gole, J. L., “Photoluminescence Induced Metallization for Low Resistance Contacts”, in Technology Advances, Invited, MRS Bulletin 2003, 28, 263.
5. Nozik, A. J. Inorg. Chem., 2005, 44, 6893-6899
6. Kottke, P. A.; Federov, A. G.; Gole, J. L. “Multiscale Mass Transport in Porous Silicon Gas Sensors,” in Modern Aspects of Electrochemistry, Vol. 43, Schlesinger M., Ed., Springer, New York, 2009, 139-168.***
7. Gole, J. L.; Goode, E. C. Laminack, W., ChemPhysChem, 2012, 13, 549-561.
8. Laminack, W.; Pouse, N.; Gole, J. L., ECS J. Solid State Sci. Tech., in press, 2012, (JSS-12-0688R).
9. Gole, J. L.; Stout, J.; Burda, C.; Lou, Y.; Chen, X. J. Phys. Chem. B 2004, 108, 1230-1240.
10. Chen, X.; Lou, Y.; Samia, A. C. S.; Burda, C.; Gole, J. L. Adv. Funct. Mater., 2005, 15, 41-49.
11. Laminack, W., Gole, J. L. “Light Enhanced Electron Transduction and Sensing at a Nanostructure Modified Semiconductor Interface” submitted.
12. See for example tables in Lévy-Clément, C. “Macro-Porous Micro-Structures including Silicon,” in Encyclopedia of Electrochemistry, in Semiconductor Electrodes and Photoelectrochemistry, Vol. 6 in Licht, S. (Ed.); Bard, A. (Series Ed.), Wiley-VCH, Weinheim, Germany, Chapter 3.2, pp. 185-237, 2002.
13. Campbell, J.; Como, J. A.; Larsen, N.; Gole, J. L.; Journal of Electrochemical Society, 2008, 155, D128 D132.
14. Pearson, R. G. Coord Chem. Rev. 1990, 100, 403-25; Pearson, R. G., “Chemical Hardness”, John Wiley VCH, Weinheim, 1997.
15. Pearson, R. G., J. Am. Chem. Soc. 1963, 85, 3533-3539. Pearson, R. G., “Chemical Hardness”, John Wiley VCH, Weinheim, 1997. Pearson, R. G., J. Chem. Sci. 2005, 117(5), 369-377. Pearson, R. G., Inorg. Chem. 1988, 27, 734-740.
16. Parr, R. G.; Pearson, R. G., J. Am. Chem. Soc. 1983, 105, 7512-7516.
17. Parr, R. G.; Yang, W. “Density Functional Theory of Atoms and Molecules,” Oxford University Press, New York, 1989.
18. Geerlings, P.; De Proft, F.; Langenaeker, W., Chem. Rev. 2003, 109, 1793-1874.
19. Laminack, W.; and Gole, J. L. to be submitted
20. Gole, J. L.; Brauer, J.; Graham, S.; Hu, J.; Dixon, D. A., “Creating Looser and More Basic Zeolite Structures from Direct Low-Temperature Nitridation”, manuscript in preparation.
21. Kumar, S.; Fedorov, A. G.; Gole, J. L., Appl. Catalysis B: Env., 2005, 57, 93-107.
22. Ozdemir, S,; J. L. Gole, Current Opinions in Solid State and Materials Science, 2007, 11, 92-100.
23. Ozdemir, S.; Gole, J. L., Sensors and Actuators B, 2010,151, 274-280.
24. Gole J. L.; Ozdemir, S.; Osburn T. S., ECS Transactions, 2010, 33 (8), 239-244.
25. Gole, J. L.; Laminack, W., “General Approach to Design and Modeling of Nanostructure Modified Semiconductor and Nanowire Interfaces for Sensor and Microreactor Applications,” in “Chemical Sensors: Simulation and Modeling, Volume 3-Solid State Sensors,” Momentum Press, New York, USA, in press.
26. Lee, E. L., and I. E. Wachs, J. Catal., 2008 258, 103-110.
27. Badlani, M.; Wachs, I. E., Catalysis Letters, 2001 Vol. 75, No. 3-4,
28. Levy-Clement, C.; Lagoubi, A.; Tomkiewicz, M, J. Electrochem. Soc., 1994, 141, 958-967.
29. Foell, F.; Christophersen, M.; Carstenson, J.; Hasse, G.; Materials Science and Engineering R280, 2002, 149.

### Example 3

The response matrix, as metal oxide nanostructure decorated n-type semiconductor interfaces are modified in situ through direct amination and through treatment with organic sulfides and thiols, is demonstrated. Nanostructured TiO₂, SnOₓ, NiO and CuₓO (x=1,2), in order of decreasing Lewis acidity, are deposited to a porous silicon interface to direct a dominant electron transduction process for reversible chemical sensing in the absence of significant chemical bond formation. The metal oxide sensing sites can be modified to decrease their Lewis acidity in a process appearing to substitute nitrogen or sulfur, providing a weak interaction to form the oxynitrides and oxysulfides. The sulfur systems likely weakly bind sulfur-based compounds. Treatment with triethylamine and diethyl sulfide decreases the Lewis acidity of the metal oxide sites. Treatment with acidic ethane thiol modifies the sensor response in an opposite sense, suggesting that there are thiol (SH) groups present on the surface that provide a Brönsted acidity to the surface. The in situ modification of the metal oxides deposited to the interface changes the reversible interaction with the analytes, NH₃ and NO. The observed change for either the more basic oxynitrides or oxysulfides or the apparent Brönsted acid sites produced from the interaction of the thiols do not represent a simple increase in surface basicity or acidity, but appear to involve a change in molecular electronic structure, which is well explained using the recently developed inverse hard and soft acid and base (IHSAB) model.

### Introduction:

There is a substantial need to develop new materials that allow the sensing of chemicals in a broad range of environments. A combination of uniquely defined active interfaces and the ability to confine processes at the nanoscale, coupled with the ability to manipulate nanostructured materials and their interactions at select interfaces, offers a special opportunity to develop economically viable, energy-efficient and sensitive modes of detection for chemical species [1-3]. The ability to manipulate and control charge transport at porous semiconductor micro/nanoporous interfaces, driven by nanostructure-focused Brönsted and Lewis acid-base chemistry, can play a major role in the development of highly responsive, sensitive (ppb), reversible sensors [4-6]. Within this framework, the creation of novel, highly active, micro-/nano-structured porous extrinsic semiconductor interfaces, their ability to provide readily accessible significant light harvesting surface areas [7] and their ability to be transformed with select nanostructure interactions [1-3] provide new avenues for sensing based on energy transfer and transduction [8]. Nanopore coated, microporous arrays not only enable enhanced Fickian diffusion [9] to active sites, but also, the nanopores provide a “phase matching” region with which modifying nanostructured materials can be made to interact in a controlled manner to promote a distinct and controllable, wide ranging and variable interface sensitivity [10].

We have recently developed a new concept, inverse hard and soft acids and bases (IHSAB) [1-3], that expands on the tenants of the HSAB [11] principle and allows the design of novel sensors, as well as catalytic sites. The IHSAB principle incorporates the coupling of analyte/interface acid-base chemistry, an approach to the balance and separation of surface physisorption (electron transduction) and chemisorption, and the ability of active nanostructures to utilize these differences. Here, the concept of electron transduction [1-3,8] is defined as the transfer of electrons to or from an interface without the formation of a chemical bond. At the heart of the concept, based on our experimental observations, is the effective transfer of electrons to acidic or from basic molecules (analytes) at a nanostructure modified extrinsic semiconductor interface. As primarily acidic metal oxides, the nanostructures focus the interaction and coupling with the majority charge carrier concentration of an extrinsic p- or n-type semiconductor, directing an electron transduction process. We have now found that these metal oxide nanostructures can be readily functionalized, in situ, to create what appear to be metal oxynitride [10,12] and oxysulfide [13] sites. The semiconductor interfaces can also be modified, as they are treated with nanostructured photocatalysts to provide a light-enhanced sensing efficiency. In concert, these concepts can be used to formulate solar pumped sensors [14]. In this manuscript, we outline the nature of the formation of nanostructure-modified reversible sensor interfaces and the nature of those changes that occur as these interfaces are modified in situ to produce oxynitride and oxysulfide sites. Our observations suggest that the observed changes can be explained within the recently developed IHSAB principle [1-3].

The fractional deposition of nanostructured metal oxide centers can be used to create inexpensive, micro-fabricated interfaces and selective interfacial platforms that can be developed for applications of a focused electron transduction built on our IHSAB model [1-3]. Nanostructured metal oxide treatments modify the interface activity to create a dominance of physisorption/weak chemisorption verses significant chemical bonding, facilitating a reversible porous silicon (PS) gas sensor response. In order to explain this behavior, we have developed a complementary concept to that formulated by Pearson et al. [11,12,13,14,15] for hard and soft acid-base (HSAB) interactions. In the HSAB concept, the interaction strength is correlated with the relative acidity and basicity of the reactants, as strong acids react with strong bases and weak acids interact with weak bases, resulting in significant ionic and covalent bonding, respectively. We wish to minimize this bond formation. A nanostructure-treated PS gas sensor can be made to behave in a physisorption/weak chemisorption dominated mode, as the IHSAB concept can be used to explain this behavior [1-3]. Here, the physisorption process is found to dominate for primarily strong acid-weak base and weak acid-strong base interactions. The emphasis is to impede bond formation by creating a molecular orbital mismatch. By assessing trends within the IHSAB framework, appropriate selections can be made for the modification of the porous Si hybrid interface with nanostructured metal/metal oxide deposits to create a range of sensitivities for a number of gases [1-6].

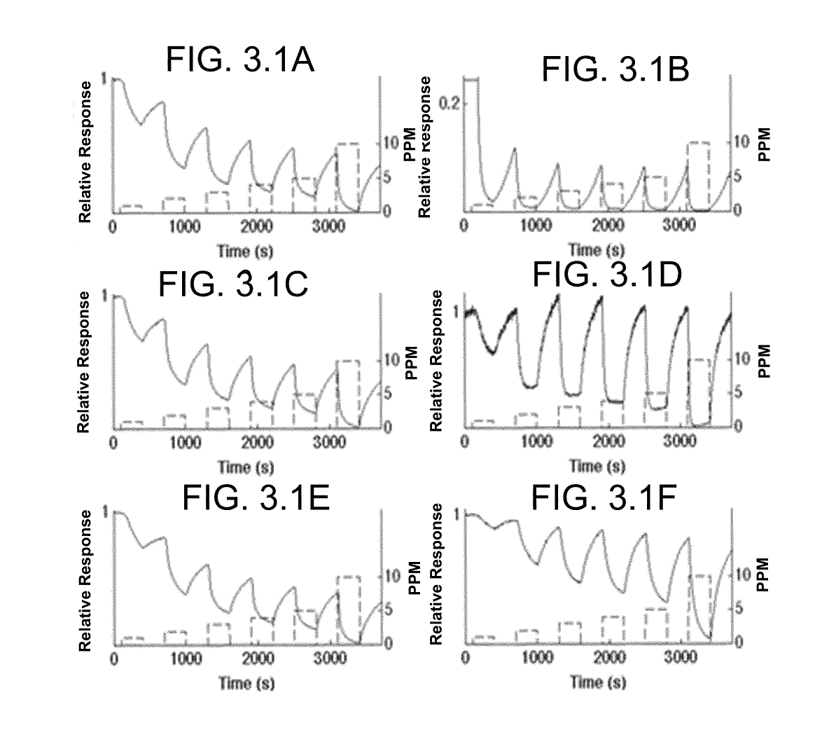

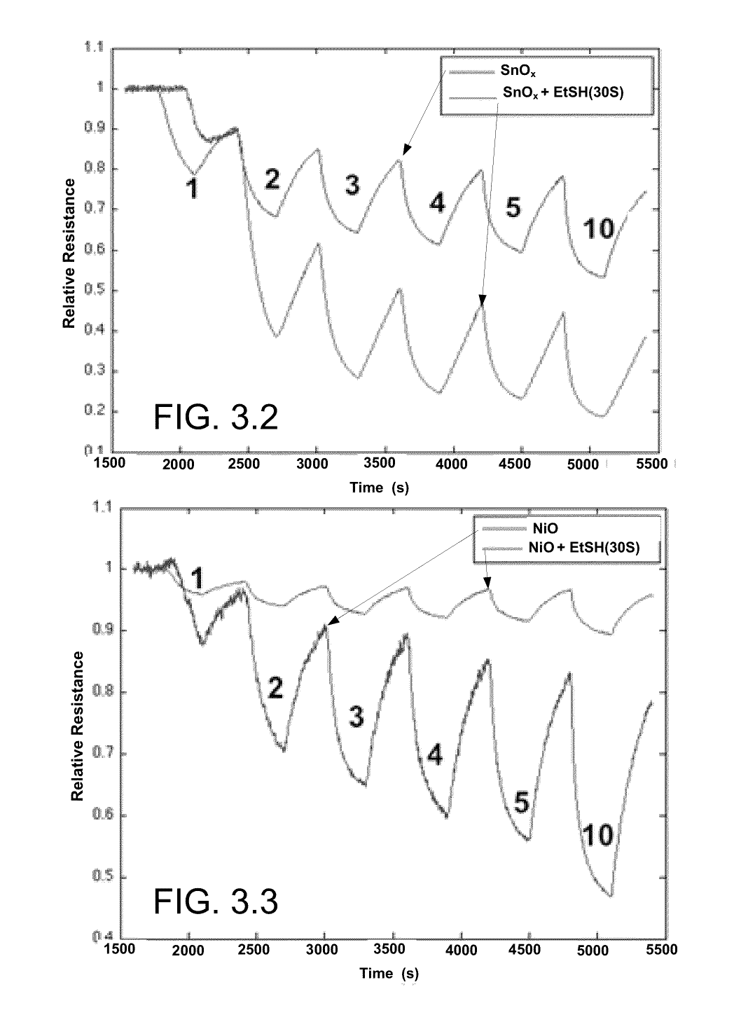

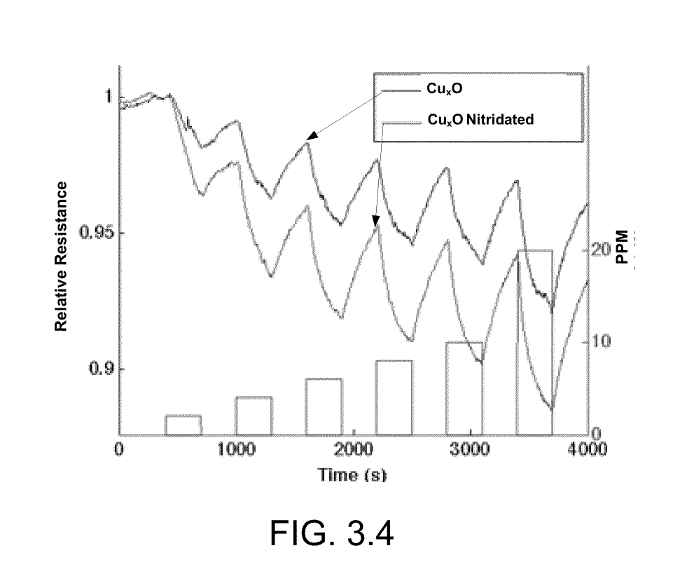

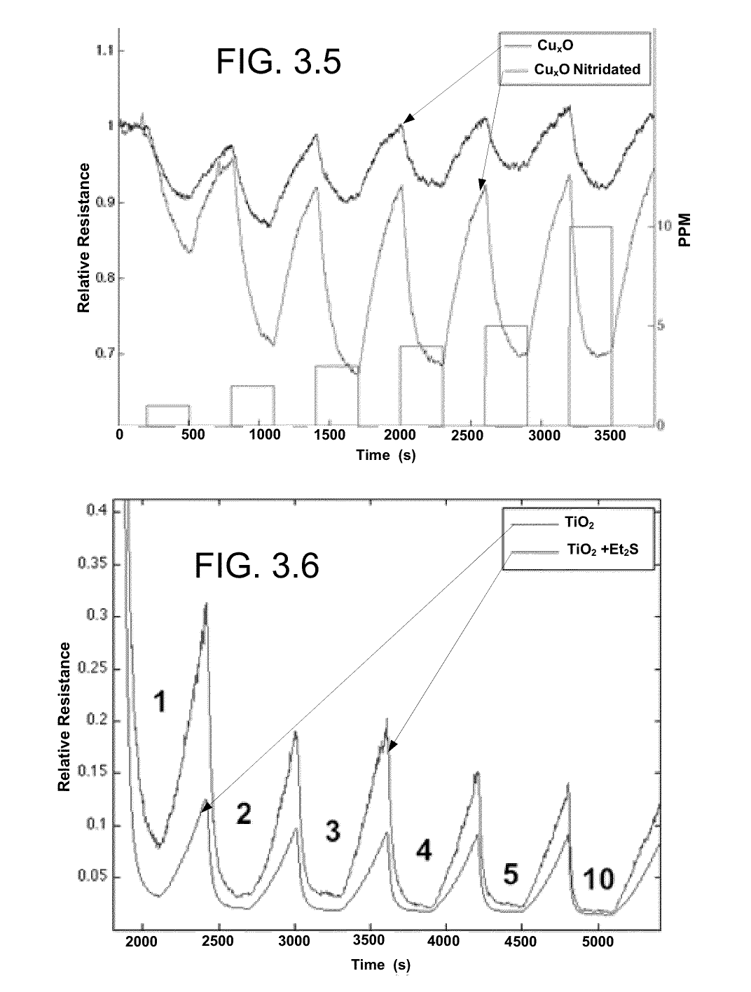

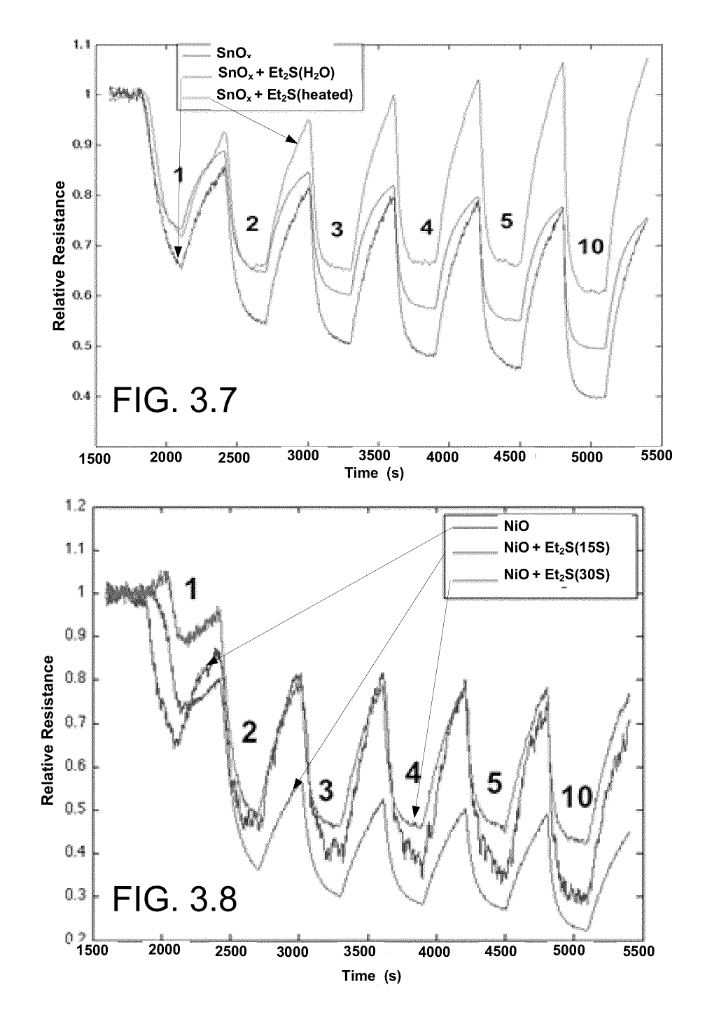

The deposition of metal oxide nanostructures introduces new selective sites, which modify the PS interface on which they are deposited. This produces an enhanced and variable response relative to an untreated interface, in direct relation to the acid strength of the deposited metal oxide, the degree of basicity or acidity of the analyte and the nature of the extrinsic semiconductor doping [1-6]. It is possible to infer the reversible interaction of NH₃ with a variety of surfaces using the IHSAB model. We exemplify this interaction for select TiO₂—, SnOₓ—, and NiO-treated PS interfaces in FIGS. 3.1A-F. NH₃, as a strong base, contributes electrons to a PS interface. For n-type PS this results in an increase in the number of excess charge carriers, which are electrons, and, as FIGS. 3.1A-F demonstrate, a decrease in resistance (increase in conductance). Titanium oxide and tin oxide nanostructure deposits, as strong acids, significantly enhance the extraction of electrons and the response of the PS interface to NH₃. In fact, by comparison, the responses saturate the conductance for NH₃ concentrations greater than 2ppm. As a stronger acid, TiO₂ is more effective. NiO, which represents an intermediate acid, also enhances the interface response, but to a lesser degree. The changes in response depicted in FIGS. 3.1A-F are predictable from the IHSAB concept. By evaluating the reversible interaction of a given analyte with the nanostructure-deposited metal oxides, it is possible to construct the acid/base interaction diagram depicted in FIG. 1.1. It is also feasible to expand the range of interface acidity by modifying the metal oxide nanostructure deposits, and we have obtained initial evidence for the facile in situ transformation of the metal oxides to their corresponding oxynitrides and oxysulfides [16,17] at the nanoscale. The systems that we have studied are summarized in Table 1.

TABLE 1
Summary of metal oxide nanostructure deposits and chemical
functionalization using nitrogen and sulfur compounds.
The systems studied and in situ exposures are given in the table.

Metal Oxide

| Chemical Dopant | TiO₂ | SnO₂ | NiO | CuₓO |
|---|---|---|---|---|
| Triethylamine, 30 s | * | * | * | |
| Thiol, 30 s | | * | * | |
| Diethyl Sulfide, 15 s | * | * | * | |

* signifies the change in response to the analyte, NH₃ as summarized in the paper.

### Results and Discussion:

In FIG. 1.1, the analyte scale is fixed in terms of acid/base properties, as determined by the energy of the lone pair (lone electron) donating to the positive metal site. The analyte lone pair energies can be evaluated from their ionization potentials or proton affinities (gas phase basicity). The sensor scale in FIG. 1.1 can be varied by substituting N or S for oxygen, as they donate electron density into the metal. There is the apparent ability for in situ transformation of the deposited metal oxide nanostructures, which can enhance the array of distinct responses that can be developed and extended to form “materials sensitivity matrices” for a given analyte, as it provides a route to decrease the Lewis acidity of these acidic sites. The degree of nitridation can be used to introduce a progressively increasing site basicity at the nanoscale [16]. This transformation is easily accomplished through direct amination in a manner analogous to that applied to the facile conversion of TiO₂ to TiO₂₋ₓNₓ [18,19,20]. The in situ formation of the oxynitrides shifts the transformed oxides toward the soft acid side of FIG. 1.1, adding breadth to this material's selectivity table.

FIGS. 1.3A and B demonstrate that while the strong (hard) acid, TiO₂, increases the sensitivity of the untreated n-type PS interface to NH₃, the oxynitride, TiO₂₋ₓNₓ, formed through in situ amination of the TiO₂-deposited surface, decreases the response to NH₃. This result is consistent with the observed effect of in situ nitridation, as it modifies the response of the sensor interface within the IHSAB format. TiO₂, as a strong acid, enhances the capture of electrons, transferring these electrons to increase conductance (decrease resistance) relative to the undecorated interface. The formation of the oxynitride decreases the metal site Lewis acidity and does not facilitate electron transduction as efficiently. The sensor response corresponds to a conductance decrease relative to the TiO₂-treated interface. The in situ nitridation of TiO₂ shifts the nature of this metal oxide nanostructure toward the soft acid side of FIG. 1.1, closer to ammonia. The IHSAB principle dictates that the response of the TiO₂₋ₓNₓ interface should decrease relative to TiO₂, as it indeed does. However, the nitridation process does not simply increase the basicity of the nanostructure surfaces. The control of the interaction with the molecule to be sensed is dictated by orbital orientation and steric effects. A weak interaction with minimal chemical bonding occurs if the donor orbital (highest occupied molecular orbital, HOMO) energy is not well matched with the acceptor (lowest occupied molecular orbital, LUMO) energy. As the HOMO (donor)-LUMO (acceptor) energy gap decreases, there will be more charge transfer and a stronger Lewis acid-base interaction. The IHSAB principle dictates that the orbital matchup and Lewis acid-base bonding with NH₃ is enhanced. By comparison, the sensor response of the TiO₂₋ₓNₓ interface decreases relative to TiO₂. In both concert and contrast to the behavior expected for a simple basic interface, the sensitivity of the weaker metal oxides can be enhanced by nitridation.

Similar decreases in the observed sensor response are observed as nitridated SnOₓ, whose Lewis acidity has decreased, interacts with NH₃ and NO, where the molecular orbital makeup is now more closely aligned [16]. FIG. 3.4 presents comparable data as 1-10 ppm of ammonia interacts with an aminated copper oxide treated n-type PS interface, converting this interface in situ to a copper oxynitride interface. Again, the nitridation of CuₓO decreases Lewis acidity and shifts the response of the modified nanostructures further to the soft acid side of FIG. 1.1. It is tempting to hypothesize that the formation of the oxynitride should simply increase the basicity of the nanostructure surface and, thus, should decrease the response to NH₃. However, this does not occur. The nitridated copper oxide is shifted further to the soft acid side of ammonia in FIG. 1.1, dictating a greater HOMO-LUMO mismatch of interacting molecular orbitals. The IHSAB principle suggests, counter to intuition, that the response of the in situ-treated nitridated copper oxide interface should increase relative to that of CuₓO, precisely as is observed. In FIG. 1.1, NO is positioned directly under the copper oxides. Nitridation shifts the copper oxides to the soft acid side and away from NO, leading to an increase in the HOMO-LUMO mismatch. As FIG. 3.5 demonstrates, the reversible response of the oxynitride to NO increases relative to that of the Cu_{X}O-decorated PS interface. The results in FIGS. 1.1 and 3.4-3.5 strongly suggest that the IHSAB principle can be used as an important distinguishing principle of sensor response and the transformation from electron transduction to chemisorption. In support of this argument, initial results obtained for the nitridation of NiO lead to a decrease in response for NO; however, as would be predicted by the IHSAB model, the reversible response for interaction with NH₃ increases [16]. The simplicity of the in situ nitridation process [10,16,18,19,20] can provide an important means of enhancing interface modification and selection.

The concept of in situ nitridation has now been extended. Initial results have been obtained for interfaces in which metal oxide nanostructures are functionalized with sulfur to form the oxysulfides. In situ treatment with R₂S sulfides lowers the Lewis acidity of the treated metal oxide site (M^{+x}) relative to the oxide and appears to convert the oxides to oxysulfides where the bonding sulfur maintains additional R groups. The substitution of sulfur for oxygen in the MO lattice would be expected to place more electron density on the M site and, hence, lower the Lewis acidity of M. In addition, an S in the lattice is softer and would have less negative charge. However, when the metal oxide interfaces are treated with the thiols, RSH, we observe the manifestation of an increase in the acidity, which suggests that there are thiol (SH) groups on the surface. In other words, we find evidence for an increase in the Brönsted acidity of the surface. The thiols are more acidic than alcohols, due to their weaker S—H bonds and the better ability of sulfur to hold negative charge after proton loss.

FIGS. 3.2, 3.3, and 3.6-3.8 demonstrate the results of the sulfur functionalization. FIG. 3.6 corresponds to the responses observed when NH₃ contributes electrons to a diethyl sulfide-treated titanium oxide-deposited PS interface. The process decreases the ability of the interface to extract electrons from NH₃ as the majority charge carrier concentration (electrons) and the conductance for the diethyl sulfide-treated, TiO₂-deposited PS interface decrease. FIG. 1.1 suggests that a decreased Lewis acidity on treatment with Et₂S produces a greater HOMO-LUMO orbital mismatch between TiO₂ and NH₃.

FIG. 3.7 corresponds to the responses observed when NH₃ contributes electrons to a diethyl sulfide, (C₂H₅)₂S, treated tin oxide-deposited PS interface. After an initial treatment of the tin oxide-deposited surface, the diethyl sulfide treatment produces a significant increase in conductance relative to the surface deposited only with tin oxide. The process appears to increase the majority charge carrier concentration (electrons) and the conductance relative to an untreated PS interface for both the diethyl sulfide-treated and tin oxide-deposited PS interfaces [17]; however, mild heating (˜80° C.) of the sulfidated surface decreases the conductance relative to the treated metal oxide-deposited surface. The conductance is now found to decrease relative to the tin oxide-deposited interface. Based on observations after treatment with EtSH (ethane thiol) that we will outline, we suggest that the decrease of conductance is likely due to the removal of interacting water previously hydrating the sulfide. The sulfide in the absence of significant water interacts with the SnOₓ nanostructure-decorated interface to produce a site of decreased Lewis acidity, diminishing the HOMO-LUMO orbital mismatch with NH₃. This is consistent with the diminished transfer of electrons from NH₃ to the sulfur-substituted SnOₓ-treated interface. In contrast, FIG. 3.7 demonstrates that the sulfidated surface can be hydrated and that this is manifest by an increase in the acidity of the surface sites relative to SnOₓ.

FIG. 3.8 corresponds to the responses observed when NH₃ contributes electrons to a diethyl sulfide-treated nickel oxide-deposited PS interface. The process increases the majority charge carrier concentration (electrons) and the conductance for both the diethyl sulfide-treated and nickel oxide-deposited (FIG. 3.8, blue line) PS interfaces. The initial treatment of the nickel oxide-deposited surface with diethyl sulfide for ten seconds produces a decrease in conductance relative to the surface deposited only with nickel oxide (Note also FIGS. 3.1A and B). However, the red curves in FIG. 3.8 demonstrate that an increase to a 15 s exposure of the diethyl sulfide results in an increase in the conductance of the sulfur-treated surface relative to the metal oxide-deposited surface. Thus, the level of diethyl sulfide exposure must be carefully assessed and optimized [1-6]. FIG. 1.1 suggests that the decreased Lewis acidity, which treatment with Et₂S produces (substitution of S for O), will lead to a greater HOMO-LUMO orbital mismatch between NH₃ and the nickel oxysulfide. This promotes a stronger reversible electron transduction with NH₃ and, thus, an enhanced conductance. FIG. 3.8 also suggests that it is possible to tune the Lewis acidity of the metal site in the oxide interface through controlled exposure to the sulfide.

In contrast to the sulfides, the thiols, RSH, are acidic rather than basic, and we find that their interaction with SnOₓ- and NiO-nanostructured oxide surfaces is the reverse of that for the sulfides. This suggests the presence of S—H bonds on the surface of the thiol-treated interface. S—H bonds on the surface of the interface emanating from the thiol can create Brönsted acid sites, which are manifest as the increased acidity of the doped metal oxide site.

FIG. 3.2 corresponds to the responses observed when NH₃ contributes electrons to an ethanethiol, CH₃CH₂SH, -treated tin oxide-deposited PS interface. The process increases the majority charge carrier concentration (electrons) and the conductance for both the ethanethiol-treated and tin oxide-deposited PS interfaces. Once optimized, the ethanethiol-treated surface displays a significant increase in conductance relative to the surface that is deposited only with tin oxide. This is consistent with the interaction of an acidic thiol that interacts with the SnOₓ nanostructure to produce a modification to the hard acid side of FIG. 1.1. This process can occur if S—H bonds are formed on the interface surface providing a Brönsted acidity. This can create an effect similar to an increased HOMO-LUMO gap, due an increase in Lewis acidity. Additional data also suggests, in complement to the sulfides, that the degree of acidity of the SnOₓ-deposited surface can be varied, increasing the magnitude of conductance, in a controlled manner.

FIG. 3.3 corresponds to the response observed when NH₃ contributes electrons to an ethanethiol-treated nickel oxide-deposited PS interface. The process results in a decrease in the conductance relative to the nickel oxide-treated surface, as it decreases the majority charge carrier concentration (electrons) and the conductance relative to the NiO-deposited interface. The effect of the thiol suggests that it increases the acidity of the NiO-treated surface as it forms Brönsted acid S—H sites. Thus, the NH₃— and thiol-treated interfaces are more closely matched with a decreased

### HOMO-LUMO Gap Separation.

The initial results, which we outline, suggest the possibility of a novel, general and readily applied approach to the formation of sulfur-functionalized interfaces, which may find application in the manipulation of biomolecules [21,22], for example, by simplifying the application of DNA oligonucleotides, that are now thiol-tagged, for surface immobilization[22]. In concert with those results obtained for nitridation, these studies also suggest the potential for extension to the remaining pnictogens, chalcogens and oxyhalides.

### Experimental Section:

Earlier, we described [9] highly efficient nanostructure modified interfaces on n-type PS, as we generated a micro-/nano-porous interface [1]. A hybrid etch procedure is used to generate the nanopore covered micropores [1,2,23,24]. The PS interface is generated by electrochemical anodization of 1-20 ohm-cm, n-type (phosphorous doped) (100) silicon wafers (Wafer World) (FIGS. 3.1A and B). The anodization of the n-type wafers [25,26] is done under topside illumination using a Blak-Ray mercury lamp. The silicon wafer is etched in a 1:1 solution of HF and ethanol at a current between 8 and 15 mA/cm [25-29]. The anodized n-type sample is placed in methanol for a short period, subsequently transferred to a dilute HF solution for a 30-minute period and, then, washed again in methanol. This process creates a porous structure with pore diameters on the order of 0.5-0.7 μm and pore depths varying from 50 to 75 μm. The micropores provide a medium for Fickian diffusion to the surface nanoporous layer.

The PS hybrid arrays of nanopore covered micropores are tested at room temperature for their individual sensor response. The nature of this response is based on the application of the IHSAB acid/base principle. The selection of the nanostructures and the variable surface sensitivities that are produced as they form in situ metal oxide deposits introduces a distinct systematics of design. The approach is unique in that the nanostructures are deposited fractionally to the PS micropores, and this fractional deposition does not require any time-consuming self-assembly within the pores. This is not a coating technique or one that requires an exacting structural film arrangement, but is, in fact, a much simpler process [1,2,23,24]. The combination of distinctly different responses observed can be used as a basis to develop selectivity. Results obtained with nanostructured deposits generated from electroless tin, nickel and copper, as well as nano-titania are considered in this study.

All of the nanostructured metals deposited to the PS surface are readily oxidized to SnOₓ (x=2,4), NiO and CuₓO (x=1,2), as demonstrated by XPS measurements [3,30]. The initially introduced nano-titania (anatase) may be crystalline, although we cannot be certain of this crystallinity after deposition to the PS interface [18].

Triethylamine (TEA) is introduced to the nanostructured metal oxides by direct in situ treatment. The metal oxide treated surface is exposed to the TEA for 10 s. The treated interface is subsequently washed in methanol to remove excess TEA and allowed to age for approximately 24 h. Sulfur is introduced to the nanostructured metal oxides through direct in situ treatment with diethyl sulfide. The metal oxide-treated surface is exposed to diethyl sulfide for 10 s. The thiols are introduced to the nanostructured metal oxides through direct in situ treatment with ethane thiol or butane thiol. The metal oxide-treated surface is exposed to the thiols for 30 s. The treated interface is subsequently washed in methanol to remove excess sulfide or thiol and allowed to age for approximately 24 h.

The sensors are evaluated in an unsaturated mode, since the time scale for reversibility may become an issue in a long-term saturated mode, and the longer term exposures are not necessary [1,2,23,24]. NH₃ was pulsed onto these interfaces with a 300 s half-cycle followed by a 300 s half-cycle nitrogen cleaning. The numbers denote ppm exposure to NH₃. The system was purged with ultrahigh purity nitrogen for 1800 s before operation. The sensor response and recovery times for “sticky gases”, such as ammonia, are distinctly different, and full time recovery from the gas exposure takes longer than 300 s. This is the exposure time duration in the present configuration (FIG. 2, [4]). However, the onset of the sensor response for these atmospheric pressure “open inlet” studies remains clearly visible. The behavior, which looks very much like the reverse of FIG. 2 in [4] suggests that the responses for NH₃ on PS are that of a gas whose interaction may be dominated by physisorption, but which also displays weak chemisorption. Purging the sensor surface with UHP N₂ for longer durations improves the gradual shift to the initial base line. The return to baseline can also be further improved by more tightly constraining the gas flow path to the sensor surface.

In all cases, the analyte gas being sensed is brought to the hybrid surface after entrainment at room temperature in UHP nitrogen (99.999%+Matheson). The typical resistances for the base PS structures range between 300 and 10,000 ohms at room temperature. The gas flow for the analyte and the entraining UHP nitrogen is controlled by MKS type 1179A mass flow controllers (MKS Instruments Andover, Mass., USA). The mass flow controllers used to control the analyte gas and the entraining nitrogen flow responded in less than 2 s. The diffusion time of the analyte gas to the sensors, which provides the longest system time constant, varies from four to five seconds for the lowest analyte concentrations, to an order of 1 to 2 s for concentrations greater than 2ppm. These are the delay times for the observation of a signal, due to the analyte in the supply line. The sensors respond to the analyte gas on a time scale much less than two seconds. The change in resistance is measured in one-second intervals using a DC current. This voltage bias used in these experiments is 3 volts, to obtain an optimum signal-to-noise ratio. An NI DAQPad-6015 (National Instruments, Austin, Tex., USA) is used for gathering data and supplying the DC current. Labview software is used to control the experiment and record the results. MATLAB is used in the analysis of the data.

### Conclusions:

The data that we have presented suggests that we can vary the acid/base properties of the metal oxide (metal centers) outlined in FIG. 1.1 and the sensor scale, varying the metal positive charge by doping in situ with sulfur and nitrogen substituted from appropriate precursors. For a TiO₂ nanoparticle site, the Ti is nominally in the +4 oxidation state. Sulfur and nitrogen will donate electron density into the metal if they are substituted for oxygen. This will shift the metal toward the softer acid side in the top portion of FIG. 1.1. Thus, the interaction with the fixed analytes catalogued in the bottom of this figure will increase, and the sensor signal will decrease. However, the change in Lewis acidity does not shift the doped TiO₂ further to the right than the fixed position of NH₃, so the signal response for NH₃ and NO will both decrease and remain of the same sign. However, for a sensing metal site between two analytes, for example, Ni²⁺, the situation is modified. Ni²⁺ is approximately equidistant between NH₃ and NO. As its Lewis acidity decreases, the signal from NO will decrease and that for NH₃ will increase. These patterns provide a basis for increasing the breadth of sensitivity matrices.

There are different ways to control the size of the interaction of those molecules that are to be sensed with variably doped metal oxide-sensing interfaces. If the orbital orientation at the surface is not correctly configured, there can be little binding with the lone pairs of the incoming molecules. In addition, a combination of molecular and surface steric effects could also block the interaction at the surface by preventing orbital overlap. If the donor orbital energy (highest occupied molecular orbital, HOMO) is not well matched with the acceptor (lowest unoccupied molecular orbital, LUMO), then the interaction will be weak. As the HOMO (donor)-LUMO (acceptor) energy gap decreases, there can be more charge transfer between the molecule and the sensor interface, leading to a stronger Lewis acid-base interaction. For Lewis acid-base bonds, the donor retains the electron pair, a prototypical example being BH₃NH₃, with a B—N bond dissociation energy (BDE) of 26 kcal/mol [31]. At the other extreme is the interaction of an anion and a cation forming an ionic bond with a much large BDE. If the electrons are fully shared, leading to the formation of a covalent bond, this can also lead to a large BDE. The IHSAB is, in large part, based on controlling the size of the Lewis acid-base bond dissociation energy.

Our results, thus, by far display a clear quantitative dependence on concentration; however, they are based on qualitative inferences from measuring the sensing signals from the interactions of molecules interacting with surfaces via donor-acceptor interaction. We intend to obtain more detailed physical measurements on the structures of the surfaces and the energetics of these surfaces. Molecular data needed to address the orbital energy arguments is available in terms of molecular proton affinities, acidities and ionization potentials, but this data are not broadly available for surfaces [32,33]. While our measurements now provide semi-qualitative data about the doped metal oxide surface sites, further experiments will help to quantify that data.

We have indicated how the fractional deposition of metal oxide nanostructures can be used as a means of obtaining distinct sensor responses that show the potential for combination in an array-based format. Within the framework of integral nanostructured island sites, the behavior of the interfaces that are generated appears to be well represented by the newly developing IHSAB model. Here, we have begun to expand the versatility inherent to the metal oxides and the range of sensor response through in situ amination, converting to the more basic oxynitrides, or through in situ interaction with the basic sulfides or acidic thiols to produce the more basic oxysulfides or their corresponding hydrogen-functionalized acidic counterparts. It is significant that these results can be obtained with a simple in situ treatment at the nanoscale.

In considering the current mode of interface preparation, we will want to better understand the change in electronic character of the interface sensing system and its interaction with an analyte as the analyte injects or removes charge from the semiconductor interface. It is apparent from the data, considered in FIGS. 1.1, 1.3, and 3.2-3.8, in combination, that the observed interactions and the conductometric response of the developed interfaces represents much more than a simple acid/base interaction.

### REFERENCES

1. Gole, J. L.; Ozdemir, S. Nanostructure directed physisorption vs. chemisorption at semiconductor interfaces: The inverse of the Hard-Soft Acid-Base (HSAB) concept. ChemPhysChem 2010, 11, 2573-2581.
2. Gole, J. L.; Goode, E. C.; Laminack, W. Nanostructure driven analyte-interface electron transduction: A general approach to sensor and microreactor design. ChemPhysChem 2012, 13, 549-561.
3. Gole, J. L.; Laminack, W. I. General Approach to Design and Modeling of Nanostructure Modified Semiconductor and Nanowire Interfaces for Sensor and Microreactor Applications. In Chemical Sensors: Simulation and Modeling, Volume 3—Solid State Sensors; Korotcenkov, G., Ed.; Momentum Press: New York, N.Y., USA, 2012; pp. 87-136 and references therein.
4. Gole, J. L.; Lewis, S. E.; de Boer, J. R.; Hesketh, P. J. Sensitive, selective, and analytical improvements to a porous silicon gas sensor. Sens. Actuators B 2005, 110, 54-65.
5. Ozdemir, S.; Gole, J. L. A phosphine detection matrix using nanostructure modified porous silicon gas sensors. Sens. Actuators B 2010, 151, 274-280.
6. Ozdemir, S.; Gole, J. L. Porous Silicon Gas Sensors for Room Temperature Detection of Ammonia and Phosphine. In Chemical Sensors 8: Chemical (Gas, Ion, Bio) Sensors and Analytical Systems; ECS Transactions: Pennington, N.J., USA, 2008; Volume 16, pp. 379-387.
7. Nozik, A. J. Exciton multiplication and relaxation dynamics in quantum dots: Applications to ultrahigh efficiency solar photon conversion. Inorg. Chem. 2005, 44, 6893-6899.
8. Laminack, W. I.; Pouse, N.; Gole, J. L. The dynamic interaction of NO₂ with a nanostructure modified porous silicon matrix: Acidity, sensor response, and the competition for donor level electrons. ECS J. Solid State Sci. Technol. 2012, 1, Q25-Q34.
9. Kottke, P. A.; Federov, A. G.; Gole, J. L. Multiscale Mass Transport in Porous Silicon Gas Sensors. In Modern Aspects of Electrochemistry; Schlesinger, M., Ed.; Springer: New York, N.Y., USA, 2009; Volume 43, pp. 139-168.
10. Gole, J. L.; Laminack, W. I. Nanostructure directed chemical sensing: The IHSAB principle and the dynamics of acid/base-interface interaction. Beilstein J. Nanotechnol. 2013, 4, 20-31.
11. Pearson, R. G. Hard and soft acids and bases—The evolution of a chemical concept. Coord. Chem. Rev. 1990, 100, 403-425.
12. Pearson, R. G. Hard and soft acids and bases. J. Am. Chem. Soc. 1963, 85, 3533-3539.
13. Pearson, R. G. Chemical Hardness; John Wiley VCH: Weinheim, Germany, 1997.
14. Pearson, R. G. Chemical hardness and density functional theory. J. Chem. Sci. 2005, 117, 369-377.
15. Pearson, R. G. Absolute electronegativity and hardness: Application to inorganic chemistry. Inorg. Chem. 1988, 27, 734-740.
16. Gole, J. L.; Laminack, W. I. Direct in-situ nitridation of nanostructured metal oxide deposited semiconductor interfaces: The formation of reversibly interacting basic sites. Advanced Functional Materials 2013, to be submitted.
17. Laminack, W. I.; Thompson, C.; Gole, J. L. Nanostructure directed chemical sensing: The IHSAB principle and sulphur functionalization through the formation of metal oxysulphide sites. Applied Surface Science 2013, to be submitted.
18. Laminack, W. I; Gole, J. L. Light enhanced electron transduction and amplified sensing at a nanostructure modified semiconductor interface. Adv. Funct. Mater. 2013, in press.
19. Gole, J. L.; Stout, J.; Burda, C.; Lou, Y.; Chen, X. Highly efficient formation of visible light tunable TiO₂₋ₓNₓ photocatalysts and their transformation at the nanoscale. J. Phys. Chem. B 2004, 108, 1230-1240.
20. Chen, X.; Lou, Y.; Samia, A. C. S.; Burda, C.; Gole, J. L. Formations of oxynitride as the photocatalytic enhancing site in nitrogen-doped titania nanocatalysts: Comparisons to a commercial nanopowder. Adv. Funct. Mater. 2005, 15, 41-49.
21. Rossi, A. M.; Wang, L.; Reipa, V.; Murphy, E. Porous silicon biosensor for detection of viruses. Biosens. Bioelectron. 2007, 23, 741-745 and references therein.
22. Laurie, J. L.; Yang, J.; Weiss, S. M. Size-dependent infiltration and optical detection of nucleic acids in nanoscale pores. IEEE Trans. Nanotechnol. 2010, 9, 596-602.
23. See for example tables in Lévy-Clément, C. Macro-Porous Micro-Structures Including Silicon. In Encyclopedia of Electrochemistry, under Semiconductor Nanostructures in Encyclopedi and Photoelectrochemistry; Licht, S., Bard, A., Eds.; Wiley-VCH: Weinheim, Germany, 2002; Volume 6, Chapter 3.2, pp. 185-237.
24. Campbell, J.; Como, J. A.; Larsen, N.; Gole, J. L. Development of porous-silicon-based active microfilters. J. Electrochem. Soc. 2008, 155, D128-D132.
25. Levy-Clement, C.; Lagoubi, A.; Tomkiewicz, M. Morphology of porous n-type silicon obtained by photoelectrochemical etching I. Correlations with material and etching parameters. J. Electrochem. Soc. 1994, 141, 958-967.
26. Foell, F.; Christophersen, M.; Carstenson, J.; Hasse, G. Formation and Application of Porous Silicon. Mater. Sci. Eng. 2002, 14, 8280.
27. Kumar, S.; Fedorov, A. G.; Gole, J. L. Photodegradation of ethylene using visible light responsive surfaces prepared from titania nanoparticle slurries. Appl. Catal. B 2005, 57, 93-107.
28. Ozdemir, S.; Gole, J. L. The potential of porous silicon gas sensors. Curr. Opin. Solid State Mater. Sci. 2007, 11, 92-100.
29. Ozdemir, S.; Osburn, T.; Gole, J. L. Nanostructure modified gas sensor detection matrix for NO transient conversion of NO to NO₂. J. Electrochem. Soc. 2011, 158, J201-J207.
30. Ozdemir, S.; Gole, J. L. Novel concept for the formation of sensitive, selective, rapidly responding conductometric sensors. ECS Trans. 2010, 33, 239-244.
31. Dixon, D. A.; Gutowski, M. Thermodynamic properties of molecular borane amines and the [BH₄⁻][NH₄⁺] salt for chemical hydrogen storage systems from ab initio electronic structure theory. J. Phys. Chem. A 2005, 109, 5129-5135.
32. Hunter, E. P.; Lias, S. G. Evaluated gas phase basicities and proton affinities of molecules: An update. J. Phys. Chem. Ref Data 1998, 27, 413-656.
33. Lias, S. G.; Bartmess, J. E.; Liebman, J. F.; Holmes, J. L.; Levin, R. D.; Mallard, W. G. Gas-phase ion and neutral thermochemistry. J. Phys. Chem. Ref Data 1988, 17, 1-861.

### Example 4

Gaseous analyte interactions on a metal oxide decorated n-type porous silicon interface show a dynamic electron transduction to and from the interface depending upon the relative strength of the gas and metal oxides. The dynamic interaction of NO with TiO₂, SnO₂, NiO CuₓO, and AuₓO, in order of decreasing acidity, demonstrates this effect. Surface interactions are also controlled by the in-situ nitridation of the oxide nanoparticles, enhancing the basicity of the decorated interface. This process changes the interaction of the interface with the analyte. The observed change to the more basic oxynitrides does not represent a simple increase in surface basicity but appears to involve a change in molecular electronic structure which is well explained by the IHSAB model.

### Introduction:

With a view to developing a general approach to sense and destroy harmful analytes, we are concerned with the prediction of the relative interactions and electron flow across rapidly responding nano-structured metal oxide and oxynitride decorated extrinsic semiconductor and nano-wire interfaces (1-10). We are concerned with the manner in which this electron flow is controlled, the manner in which the electron flow couples to the semiconductor's majority charge carriers, and the variable response that this combination creates. In this way, we are constructing a designed MEMS/NEMS framework and procedure that selects specific, sensitive (ppb), and selective sensors, operable at room temperature, at atmospheric pressure, and through a broad temperature range.

We have recently implemented the IHSAB concept as a means of linking chemical selectivity and the mechanism of sensor response for both doped semiconductor and nanowire sensors (1-7). This model provides a simple-to-use prescription for design which relates, rationally, the physics and chemistry of specific nanostructure interfaces in microporous extrinsic semiconductor channels. The model combines the basic tenants of acid/base chemistry (the ability of bases to donate electrons and acids to seek electrons) and semiconductor physics so as to form a road map for the implementation of readily constructed, cost effective, rapidly responding deployable devices sensitive to the ppb level. The mechanism of selectivity relies on the use of a nanopore coated microchannel array which combines optimized analyte diffusion with maximum interface interaction. The nanoporous coating of the microchannel provides a unique phase match for the subsequent fractional deposition of select nanostructure islands that decorate the microchannel. The materials selected for the nanostructured islands serve the role of guiding antennas to force a dominant electron transduction (vs. chemisorption) at the decorated extrinsic semiconductor interface. The selection of these nanostructures and the variable and controllable physisorbed (reversible) interaction they introduce for sensor applications is well predicted by the IHSAB model as it dictates the coupling of analyte/interface acid-base interactions with the properties of the majority carriers in an extrinsic semiconductor (1-7). The selection of the nanostructures that are deposited to the nanopore covered microchannels and the variable surface sensitivities that are produced, as they form in-situ metal/metal oxide deposits which can be transformed in-situ to the respective oxynitrides, can now be predicated not in a random fashion or based on limited previous observations but in a clearly designed procedure based on established molecular properties. The nanostructures are deposited fractionally to semiconductor micropores and this fractional deposition DOES NOT require any time consuming self-assembly within the pores, and is far simpler to implement than traditional thin film or alternate coating techniques. The constraints/possibilities obtained by combining acid/base concepts and semiconductor theory offer an exciting approach whose mechanistic details are being carefully assessed in order to enable the selection of device materials targeting the sensing of specific gas analytes. The degree to which this combination allows the tailoring of interfaces through an understanding of their physics and chemistry offers the means to construct a powerful tool for analyte sensing. The extension of this approach to a much broader range of extrinsic semiconductor interfaces is extremely promising as is the ability of this developing concept to explain the response of a wide variety of semiconductor materials, including treated nanowires.

### the Sensor Interface:

### Micro/Nano-Porous Semiconductor Surface:

The semiconductor interfaces of interest are illustrated by the porous silicon (PS) nano/microporous interface depicted in FIG. 2.1. This structure is produced by a hybrid etch procedure used to create the desired interfacial support structure. The silicon structure or layers and the porous silicon (PS) structures, devices, and methods, considered in the following discussion can be replaced with any alternate extrinsic semiconductor (e.g., GaP, InP, CdTe) onto which a porous microstructure can be generated (11). Further, the configuration in FIG. 2.1 can be adapted to a pass-through microporous membrane. (12) This greatly broadens the range of nanostructure-interface combinations which can be potentially exploited as the alternate extrinsic semiconductors can be a p-type substrate, a p⁺-type substrate, or an n-type substrate. The nanopore covered microporous structure of the interface has been created specifically to facilitate efficient gaseous diffusion (Fickian) to the highly active nanostructure (FIG. 2.1) modified nanoporous (FIG. 2.1) coating (6). The nanoporous coating acts to provide a phase matching for the subsequent deposition of selected nanostructured islands (FIG. 2.1-red). These surface-attached nanoparticles possess unique size dependent and electronic structure properties that form a basis for changing the sensitivity for exposure to specific gases. This exposure alters the conductivity of the porous silicon or an alternate extrinsic semiconductor (measured by microprobe circuitry) attached to the gold contacts shown in FIG. 2.1. While the room temperature operation of this interface serves many applications, the appropriate installation of heat sinks allows operation at several hundred degrees C. A developed interface thus operates under conditions which are not amenable to typical metal oxide systems. Especially when coated with nanoparticle photo-catalysts, the interface could also operate as a nanostructure-modified microreactor for efficient chemical transformation. When operated in the electron transduction mode, the transfer of electrons to an n-type PS interface, as would occur with a basic analyte, increases the majority charge carriers which are electrons, decreases the conductmetric resistance and increases conductance. The removal of electrons, as would occur with an acidic analyte, decreases the majority charge carriers and the conductance and increases resistance. The opposite behavior will be observed for a p-type semiconductor interface.

### Inverse Hard and Soft Acid/Base Concept:

We have applied the inverse hard and soft acid and base (IHSAB) concept, using metal oxide and oxynitride nanostructure modified interfaces (1,6,13). This concept complements the tenants of HSAB interactions (14). It includes the coupling of analyte/interface acid-base chemistry with select interfaces, leading to a balance and separation of surface physisorption and chemisorption, and enables the ability of active nanostructure-based sites to utilize these differences. Based on the reversible interaction of hard acids and bases with soft bases and acids, the IHSAB principle enables the selection of interacting materials that do not form strong covalent or ionic chemical bonds and complements the HSAB model for significant bond formation based on strong ionic (hard acid/base) or covalent (soft acid/base) interactions and chemical bond formation. (14) As an extrapolation of the HSAB concept developed by Pearson and later correlated within the context of density functional theory (DFT) by Pearson, Parr, and others, the IHSAB model is somewhat broader-based and predicts reversible sensor-analyte interactions (14,15,16,17,18).

The study of interactions that allow the construction of the relative positioning of the acids and bases within the IHSAB and HSAB concepts is summarized in FIG. 1.1. Analyte response data forms the basis for the development of an initial materials positioning diagram based largely on the interaction of the acidic metal oxides which range from TiO₂ to AuₓO (x>>>1) and the bases NH₃ to CO. The relative separation of the oxides and bases dictates the observed responses of the interface. NH₃ displays a maximum reversible response for an AuₓO deposited surface whereas CO displays a maximum response for TiO₂ and SnO₂. In contrast to chemical bond formation, the greatest reversible response corresponds to the largest molecular orbital mismatch. The nitridation of the metal oxides can be used to modify the site basicity through in-situ transformation to the corresponding oxynitrides. The degree of nitridation can be used to introduce progressively increasing site basicity. The transformation is accomplished in a manner analogous to that applied to the facile conversion of nanostructured TiO₂ to TiO₂₋ₓNₓ (19). The in-situ formation of the oxynitrides shifts the positioning of the oxides toward the soft acid side of FIG. 1.1 as it promotes the formation of basic sites. This promotes a significant change in sensor response.

Nitridation Concept and Enhanced Basicity Associated with the Formation of Oxynitride Surfaces.

The enhanced basicity inherent to the oxynitride systems that we are developing can be demonstrated in multiple ways. We find consistent results obtained not only from the measurement of the in-situ change in response resulting from nitridation as predicted by the IHSAB concept and its correlation with an enhanced basic character, but also gauged by the softening of acidity. By examining the surface chemistry of nitridated nanostructures, applying the decomposition reaction of methanol, it is possible to distinguish acid and base sites and therefore the transformation from acidic to basic sites. These studies also define a broadened interaction matrix as it extends from physisorption (sensing) applications to chemisorption and microreactor design.

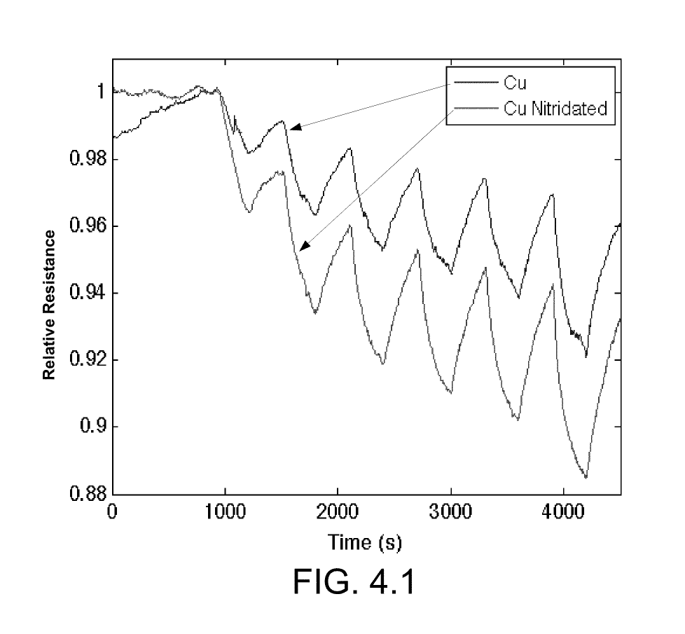

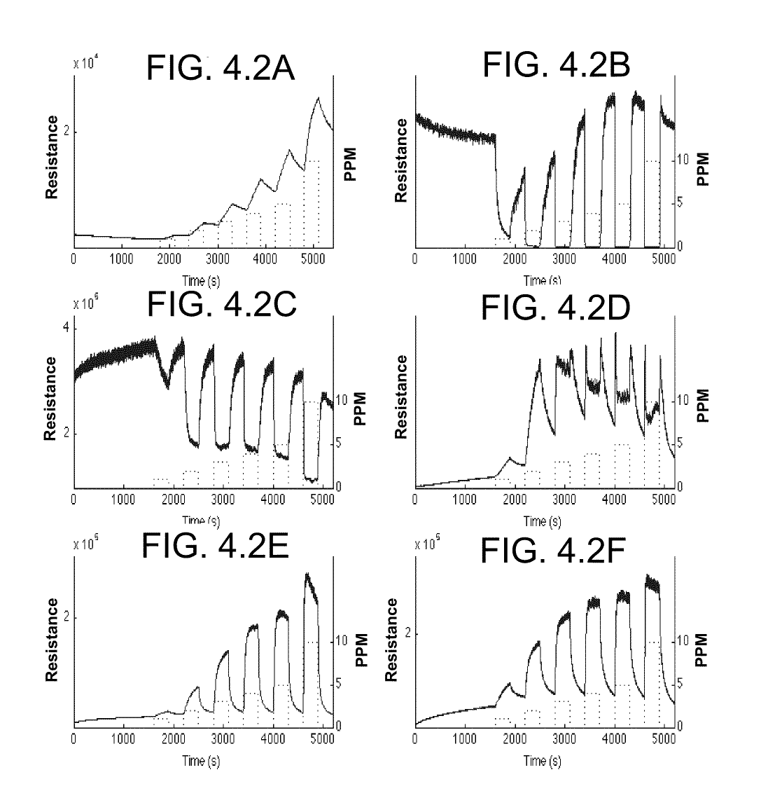

Recently, we have produced visible light absorbing TiO₂₋ₓNₓ photocatalyst nanoparticles in seconds at room temperature using alkyl ammonium compounds, leading to the direct nitridation of highly porous TiO₂ nanocolloids (19-22). The observed effect of in-situ nitridation as it modifies the response of a semiconductor interface within the IHSAB format can be demonstrated for nanostructured TiO₂ which represents a strong (hard) acid. Its oxynitride, TiO₂₋ₓNₓ, once formed, through in-situ treatment of a TiO₂ deposited surface, has gained considerable basic character (1-7). The data in FIG. 1.3 compare the response of an untreated n-type PS interface, upon exposure to 2-10 and 20 ppm of NH₃, and that for the interface treated with a deposition of “acidic” TiO₂ nanostructures, and this same interface where the deposited nanostructures are converted in-situ from TiO₂ to the more basic TiO₂₋ₓNₓ. TiO₂, as a strong acid, enhances the capture of electrons, transferring these electrons to increase conductance (decrease resistance) relative to the undecorated interface. The more basic oxynitride does not facilitate electron transduction as efficiently and the sensor response corresponds to a conductance decrease relative to the untreated interface. Note also that the in-situ nitridation of TiO₂ shifts the nature of this metal oxide nanostructure toward the soft acid side of FIG. 1.1, closer to ammonia. The IHSAB principle dictates¹⁻⁷ that the response of the TiO₂₋ₓNₓ interface should decrease relative to TiO₂ as it indeed does. Similar decreases in the observed sensor response are observed as nitridated SnO₂ interacts with NH₃ and NO with which it is now more closely aligned. The nitridation of NiO also leads to a decrease in response. FIG. 4.1 shows comparable data as 1-10 ppm ammonia interacts with a copper oxide treated n-type PS interface converted in-situ to a nitridated copper oxide interface. Again, the nitridation of CuₓO forms a more basic interface and shifts the response of the modified nanostructure further to the soft acid side of FIG. 1.1. It is tempting to suggest that the formation of the oxynitride should simply increase the basicity of the nanostructure surface and thus should decrease the response to NH₃. However, the nitridated copper oxide is shifted further from ammonia in FIG. 1.1. The IHSAB principle dictates, counter to intuition, that the response of the in-situ treated nitridated copper oxide interface should increase relative to that of CuₓO, precisely as is observed. In FIG. 1.1 NO is positioned directly under the copper oxides. Nitridation shifts the copper oxides to the soft acid side and away from NO, leading to an increase in molecular orbital mismatch and the reversible response of the oxynitride to NO. These results strongly suggest that the IHSAB concept can be used as an important distinguishing principle of sensor response.

### Microcatalysis of Metal Oxide and Oxynitride Surfaces:

We have examined several of the metal oxide and oxynitride samples for the qualitative aspects of their surface chemistry using the methanol decomposition reaction. This reaction is not a replacement for titrations with model acid and base compounds but has been effectively used by Wachs et al. to characterize the surface of bifunctional, mixed metal oxides (23,24). They have demonstrated the utility of evaluating redox, acid, and base sites on surface opened reaction manifolds leading to the products: formaldehyde, dimethyl ether, and CO/CO₂, respectively.

2CH₃OH→CH₃OCH₃+H₂O: acid sites [1]

CH₃OH→HCHO+H₂: redox sites [2]

CH₃OH→CO+2H₂: base sites [3]

Using these probe reactions, we have discerned that the nitridation process offers the opportunity to convert the metal oxide acid sites to metal oxynitride surface sites.

Dynamics of Nanostructured Metal Oxide Analyte Interaction:

We have previously considered the dynamic interaction and competition between NO₂ and a TiO₂ nanostructure modified n-type PS interface. The interaction of NO₂, which extracts electrons, leads to a resistance increase (conductance decrease). However, a fractionally deposited strong acid such as TiO₂ can compete effectively with the moderately strong acid, NO₂, for the available electrons in this system (1,13). As NO₂ is introduced to the decorated PS interface and attempts to extract electrons, the sensor resistance rises rapidly to a point when the electron depletion reaches a limiting value as nanostructured TiO₂ islands coupled to the PS interface prevent further electron withdrawal, and reverse the flow of electrons so as to increase the donor and conduction level electron concentrations. This can lead to a sharp decrease in the resistance. The process of interaction is a dynamic one as TiO₂ and NO₂ vie for the available electrons as the NO₂ is introduced and removed from the system. Further the process of electron withdrawal is strongly influenced by the relative concentration of the TiO₂ island sites. A similar dynamic playoff is observed with the amphoteric NO radical which can be either a weak acid or a weak base (6,7).

FIG. 4.2A which shows a positive resistance change with concentration indicates that, when brought in contact with an n-type PS interface, NO acts like a weak acid. The boxes in the figure indicate the concentration range from 1 to 5 ppm. Nanostructure treated PS interfaces deposited with the acidic metal oxides in decreasing strength TiO₂>SnOₓ>NiO>CuₓO>AuₓO (x>>1) demonstrate not only a starkly different response but also clear trends which can be associated with the relative acid strengths of the metal oxides. FIGS. 4.2B and 4.2C indicate the responses for the PS interfaces decorated with TiO₂ and SnOₓ. The strong acid character of TiO₂ and to a lesser degree SnO₂ has overcome the ability of NO to extract electrons. Instead, electrons are extracted from NO and transferred to the PS interface to greatly increase conductance. Although the observed responses are virtually linear to 5 ppm for the untreated PS interface, they are quenched for the TiO₂ and SnO₂ surfaces at NO concentrations in excess of 4 ppm. The observed response at 10 ppm diminishes for TiO₂ which suggests that the ability of this interface to extract electrons from NO has reached a limiting value. This is less apparent for SnO₂ but the ability of the interface to extract electrons is still significantly diminished. The magnitude of the signals observed for the TiO₂ and SnO₂ interfaces, while opposite in sense, exceed those for the untreated PS interface by factors of 12 and 2 respectively (6,7).

FIG. 4.2D corresponds to the response of a PS interface treated with the intermediate acid NiO. The response displays an intriguing intermediate behavior as NO and the decorated PS interface now competes effectively for electrons. At the lowest NO concentrations the response of the n-type PS interface is enhanced as the ratio of the responses (2ppm/1 ppm) increases significantly relative to the n-type PS as the observed response rises to a maximum throughout the cycle of NO exposure. At an NO concentration in excess of 3 ppm, the dynamic response at first increases rapidly to a sharp maximum, subsequently decreases, oscillates, and then increases as the NO concentration decreases. The dynamic behavior is even more pronounced at 4, 5, and 10 ppm. As the NO concentration increases, the transfer of electrons to NO increases to a maximum, indicated by the onset of the spike-like features which diminish in width with increasing NO concentration. We suggest that this results as the transfer of electrons to NO reaches a limit and when the depleted n-type PS interface is sufficiently depleted it acts as a stronger acid than the NO radical. At this point electrons are extracted from NO (acting as a base), accompanied by a decrease in the measured dynamic resistance (increase in conductance) as the semiconductor donor levels are repopulated. The oscillatory behavior which is especially apparent at concentrations of 4, 5, and 10 ppm is suggested to result from a continual if not less pronounced change of the competing interface and NO. This will be the subject of further study. The time dependent competition between NO and the NiO decorated interface means that the sensors return slowly to a baseline response.

FIGS. 4.2E and 4.2F demonstrate a clear trend in the responses of the weaker acid nanostructure deposited PS interfaces, based on CuₓO and AuₓO nanostructure depositions, and increase the response of the PS interface by factors of 1.2 to 1.5, respectively (6,7). The increase in response is, as expected, greater for the weaker acid AuₓO. The CuₓO and AuₓO decorated interfaces thus act to enhance the electron withdrawing power of the NO radical, which suggests that they represent weaker acids on the n-type PS interface. Consistent with trends in the acid strength of the nanostructure deposits, the CuₓO response increases to a maximum with NO exposure for concentrations 1-4 ppm, however, this increase is slowed at 4 ppm. At 5 ppm the response peaks at an intermediate time of the NO exposure and at 10 ppm, the signal peaks shortly after the NO exposure and, counter to the behavior at lower pressures, decreases. This overall behavior is consistent with the weaker acid nature of CuₓO vs. NiO. At 4 ppm, the CuₓO decorated interface begins to compete effectively with NO for the available electrons and at 10 ppm the interface is able to overcome the extraction of electrons by NO as the response shows a decrease with the time of NO exposure. Note also the almost complete return to baseline.

The trends that we describe for CuₓO extend to the weaker acid AuₓO decorated interface. Here, FIG. 4.2F demonstrates that AuₓO response increases to a maximum with exposure to NO concentrations 1-4 ppm. At 5 ppm the response peaks at an intermediate response of the NO exposure. However, at 10 ppm the response decreases with NO exposure but at a much slower rate than does the response for CuₓO. The extraction of electrons by NO is overcome but to a much lesser extent. Note also the return to baseline.

The variations in response observed for the nanostructure treated PS interfaces, while reflecting the donor level population, demonstrate the important role played by the deposited nanostructures and the nature of acid/base interaction they direct. The observed trends also correlate well with the relative responses observed as NO interacts with p-type PS. Within this framework the behavior of the NiO decorated interface is indeed intriguing. At the lowest concentrations the NiO nanostructure deposited interface mimics the dynamic behavior of the weak acids CuₓO and AuₓO and at higher concentrations it is transmuted to a surface similar to that treated with the stronger nanostructured acids TiO₂ and SnOₓ. As a function of concentration, NO acts as both an acid and a base.

### Conclusion:

We have demonstrated the efficacy of fractional nanostructure depositions as a means of obtaining distinct sensor responses which show the potential for combination in an array based format. The behavior of these systems appears to be well represented by the newly developing IHSAB model. We have also considered the conversion of the metal oxides in-situ to their oxynitrides and the enhanced basicity that this introduces to a nanostructure decorated PS interface.

### REFERENCES

1. J. L. Gole and S. Ozdemir, Chem. Phys. Chem., 11, 2573-2581 (2010). (b) R. Smith, Electrochem. Solid-State Lett., 10, A1 (2007).
2. S. Ozdemir and J. L. Gole, Current Opinions in Solid State and Materials Science, 11, 92-100 (2007).
3. S. Ozdemir and J. L. Gole, Sensors and Actuators B, 151, 274-280 (2010)
4. P. A. Kottke, A. G. Fedorov, and J. L. Gole, in Modern Aspects of Electrochemistry, M. Schlesinger editor, Vol. 43, pp 139-168, Springer. (b) S. Ozdemir and J. L. Gole, ECS Transactions, 33 (8), 111-115 (2010).
5. J. L. Gole, S. Ozdemir, and T. S. Osburn, ECS Transactions, 33 (8), 239-244 (2010).
6. J. L. Gole. E. C. Goode, and W. Laminack, ChemPhysChem, 13, 549-561. (2012).
7. J. L. Gole and W. Laminack in “Chemical Sensors: Simulation and Modeling, Volume 3—Solid State Sensors,” Momentum Press, New York, USA, in press.
8. H. Li, J. Xu, Y. Zhu, X. Chen, Q. Xiang, Talanta, 82, 458-463 (2010).
9. S. S. Kim, J. Y. Park, S-W Choi, H. S. Kim, H. G. Na, J. C. Yang, and H. W. Kim, Nanotechnology, 1-7, (2010).
10. A. Kolmakov, D. O. Klenov, Y. Lilach, S. Stemmer, and M. Moskovitz, Nanoletters, 5, 667 (2005)
11. C. Lévy-Clément, in Encyclopedia of Electrochemistry, in Semiconductor Electrodes and Photoelectrochemistry, Vol. 6 in Licht, S. (Ed.); Bard, A. (Series Ed.), Wiley-VCH, Weinheim, Germany, Chapter 3.2, pp. 185-237, (2002).
12. J. Campbell, J. A. Como, N. Larsen, and J. L. Gole, Journal of Electrochemical Society, 155, D128-D132 (2008).
13. W. I. Laminack, N. Pouse, and J. L. Gole, ECS J. Solid State Sci. Tech., 1, Q25-Q34 (2012)
14. R. G. Pearson, Coord. Chem. Rev., 100, 403-25 (1990); (b) R. G. Pearson, “Chemical Hardness”, John Wiley VCH, Weinheim, (1997).
15. R. G. Pearson, J. Am. Chem. Soc., 85, 3533-3539 (1963). (b) R. G. Pearson, J. Chem. Sci., 117(5), 369-377 (2005). (c) R. G. Pearson, Inorg. Chem. 27, 734-740 (1988).
16. R. G. Parr and R. G. Pearson, J. Am. Chem. Soc., 105, 7512-7516 (1983).
17. R. G. Parr and W. Yang, in “Density Functional Theory of Atoms and Molecules,” Oxford University Press, New York, (1989).
18. P. Geerlings, F. De Proft, and W. Langenaeker, Chem. Rev., 109, 1793-1874 (2003).
19. J. L. Gole, J. Stout, C. Burda, Y. Lou, and X. Chen, J. Phys. Chem. B 108, 1230-1240 (2004).
20. J. L. Gole, J. Brauer, S. Graham, J. Hu, and D. A. Dixon, “Creating Looser and More Basic Zeolite Structures from Direct Low-Temperature Nitridation”, manuscript in preparation.
21. X. Chen, Y. Lou, A. C. S. Samia, C. Burda, and J. L. Gole, Adv. Funct. Mater., 15, 41-49 (2005).
22. S. Kumar, A. G. Fedorov, and J. L. Gole, Appl. Catalysis B: Env., 57, 93-107 (2005).
23. E. L. Lee, and I. E. Wachs, J. Catal., 258, 103-110 (2008).
24. M. Badlani and I. E. Wachs, Catalysis Letters Vol. 75, No. 3-4, (2001)
25. J. I. Gole and W. Laminack Beilstein J. Nanotechnol. 4, 20-31. (2013)

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding based on numerical value and measurement techniques. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A method, comprising:
providing a conductometric porous silicon gas sensor including a silicon substrate having a porous silicon layer, wherein a plurality of metal oxide nanostructures are disposed on a portion of the porous silicon layer to provide a fractional coverage on the porous silicon layer, and
functionalizing, in situ, the metal oxide nanostructures with nitrogen, sulfide, or thiol to form a in situ functionalized metal oxide nanostructures.

2. The method of claim 1, wherein functionalizing, in situ, the metal oxide nanostructures includes exposing a functionalization agent to the metal oxide nanostructures to form the in situ functionalized metal oxide nanostructures.

3. The method of claim 2, wherein the functionalization agent is selected from the group consisting of: triethylamine, tributylamine, aryl amines and a combination thereof.

4. The method of claim 2, wherein the functionalization agent is selected from the group consisting of: diethyl sulfide, dibutylsufide, dimethylsulfide, and a combination thereof.

5. The method of claim 2, wherein the functionalization agent is selected ethane thiol or butane thiol.

6. The method of claim 5, wherein functionalizing includes forming thiol compounds of the sulfides or thiols maintains S—R or S—H—R groups on the surface of the metal oxide nanostructures, wherein R is an alkyl group.

7. The method of claim 2, wherein exposing includes exposing the metal oxide nanostructures to the functionalization agent for about 5 second to 60 seconds.

8. The method of claim 2, wherein the metal oxide nanostructure is selected from the group consisting of: aluminum oxide, silicon oxide, tin oxide, chromia, iron oxide, nickel oxide, silver oxide, cobalt oxide, zinc oxide, platinum oxide, palladium oxide, vanadium oxide, molybdenum oxide, lead oxide, titanium oxide, gold oxide, copper oxide, and a combination thereof.

9. The method of claim 1, wherein the metal oxide nanostructures are formed on the porous silicon layer through in situ oxidization of metal nanostructures disposed on the porous silicon layer.

10. The method of claim 1, wherein silicon substrate is a n-type silicon substrate.

11. A structure formed by the following process:
providing a conductometric porous silicon gas sensor including a silicon substrate having a porous silicon layer, wherein a plurality of metal oxide nanostructures are disposed on a portion of the porous silicon layer to provide a fractional coverage on the porous silicon layer, and
functionalizing, in situ, the metal oxide nanostructures with nitrogen, sulfide, or thiol to form a in situ functionalized metal oxide nanostructures.

12. A device, comprising:
a conductometric porous silicon gas sensor including a silicon substrate having a porous silicon layer, wherein a plurality of in situ functionalized metal oxide nanostructures are on a portion of the porous silicon layer to provide a fractional coverage on the porous silicon layer, wherein the in situ functionalized metal oxide nanostructures are functionalized in situ with nitrogen, sulfide, or thiol, wherein the conductometric porous silicon gas sensor is operative to transduce the presence of a gas into an impedance change, wherein the impedance change correlates to the gas concentration.

13. The device of claim 12, wherein the in situ functionalized metal oxide nanostructures are more basic relative to unfunctionalized metal oxide nanostructures.

14. The device of claim 12, wherein the in situ functionalized metal oxide nanostructures formed from thiol groups intereacting with and functionalizing in situ, the metal oxide nanostructures.

15. The device of claim 14, wherein the in situ functionalized metal oxide nanostructures are more acidic relative to the unfunctionalized metal oxide nanostructures.

16. The device of claim 12, wherein the in situ functionalized metal oxide nanostructure includes a metal oxide that is selected from the group consisting of: aluminum oxide, silicon oxide, tin oxide, chromia, iron oxide, nickel oxide, silver oxide, cobalt oxide, zinc oxide, platinum oxide, palladium oxide, vanadium oxide, molybdenum oxide, lead oxide, titanium oxide, gold oxide, copper oxide, and a combination thereof.

17. A method of detecting a concentration of a gas, comprising:
providing a conductometric porous silicon gas sensor including a silicon substrate having a porous silicon layer, wherein a plurality of in situ functionalized metal oxide nanostructures are on a portion of the porous silicon layer to provide a fractional coverage on the porous silicon layer, wherein the in situ functionalized metal oxide nanostructures are functionalized in situ with nitrogen, sulfide, or thiol, wherein the conductometric porous silicon gas sensor is operative to transduce the presence of a gas into an impedance change, wherein the impedance change correlates to the gas concentration:
introducing the gas to the sensor; and
measuring an impedance change in the sensor.

18. The method of claim 17, wherein the in situ functionalized metal oxide nanostructures are more basic relative to unfunctionalized metal oxide nanostructures.

19. The method of claim 17, wherein the in situ functionalized metal oxide nanostructures are more acidic relative to unfunctionalized metal oxide nanostructures.
